(19) United States
(12) Patent Application Publication
(10) Pub. No.: US 2022/0010353 A1
(43) Pub. Date: Jan. 13, 2022

(54) NUCLEASE-ASSOCIATED END SIGNATURE ANALYSIS FOR CELL-FREE NUCLEIC ACIDS

(71) Applicants: The Chinese University of Hong Kong, Shatin (HK); GRAIL, Inc., Menlo Park, CA (US)

(72) Inventors: Yuk-Ming Dennis Lo, Homantin (CN); Rossa Wai Kwun Chiu, Shatin (CN); Kwan Chee Chan, Mei Foo Sun Chuen (CN); Peiyong Jiang, Pak Shek Kok (CN); Wing Yan Chan, Tai Po (CN); Wai Kei Lam, Kowloon (CN); Diana Siao Cheng Han, Tai Po (CN); Wenlei Peng, Shatin (CN); Chen Ding, Shatin (CN)

(21) Appl. No.: 17/374,691

(22) Filed: Jul. 13, 2021

Related U.S. Application Data
(60) Provisional application No. 63/051,268, filed on Jul. 13, 2020.

(51) Int. Cl. C12Q 1/34; C12Q 1/6869

(52) U.S. Cl. CPC C12Q 1/34; C12Q 1/6869; C12Y 301/30; C12Y 301/25; C12Q 2600/112; C12Y 301/11001; C12Y 301/13001; C12Y 301/11002; C12Y 301/21001; C12Y 301/22001

(57) ABSTRACT

Various embodiments are directed to using nuclease expression in tissues that influences cell-free DNA end signatures/motifs and size of overhang between DNA strands. Embodiments can identify a nuclease that is being differentially regulated in abnormal cells relative to normal cells. Embodiments can determine that the nuclease preferentially cuts DNA into DNA molecules having: (i) a particular sequence end signature; or (ii) a specified length of overhang between a first strand and a second strand. A parameter can be determined for a biological sample based on an amount of DNA molecules that include an end sequence corresponding to the particular sequence end signature and/or a measured property correlating to the specified length of overhang. The parameter can be used to determine a characteristic of a tissue type, a fractional concentration of clinically-relevant DNA molecules, or a level of abnormality of a tissue type in the biological sample.

### CROSS-REFERENCES TO RELATED APPLICATIONS

This application claims priority to U.S. Provisional Patent Application No. 63/051,268, entitled “Nuclease-Associated End Signature Analysis For Cell-Free Nucleic Acids,” filed on Jul. 13, 2020, the contents of which are hereby incorporated by reference in their entirety for all purposes.

### BACKGROUND

Cell-free DNA (cfDNA) is a rich source of information that can be applied to the diagnosis and prognostication of many physiological and pathological conditions such as pregnancy and cancer (Chan, K. C. A. et al. (2017), New England Journal of Medicine 377, 513-522; Chiu, R. W. K. et al. (2008), Proceedings of the National Academy of Sciences of the United States of America 105, 20458-20463; Lo, Y. M. D. et al., (1997), The Lancet 350, 485-487). Though circulating cfDNA is now commonly used as a non-invasive biomarker and is known to circulate in the form of short fragments, the physiological factors governing the fragmentation and molecular profile of cfDNA remain elusive.

Recent works have suggested that the fragmentation of cfDNA is a non-random process associated with the positioning of nucleosomes (Chandrananda, D. et al., (2015), BMC Medical Genomics 8, 29; Ivanov, M. et al., (2015), BMC genomics 16, 51; Lo, Y. M. D. et al. (2010), Science Translational Medicine 2, 61ra91-61ra91; Snyder, M. W. et al., (2016), Cell 164, 57-68; Sun, K. et al., (2019), Genome Research 29, 418-427)). Previously, we have demonstrated that the Deoxyribonuclease 1 Like 3 (DNASE1L3) nuclease contributes to the size profile of cfDNA in plasma (Serpas, L. et al. (2019), Proceedings of the National Academy of Sciences 116, 641-649). Despite the above, many techniques for analyzing nuclease expression levels involve RNA sequencing or other type of RNA analyses (e.g., reverse transcriptase polymerase chain reaction). However, these RNA-based techniques can suffer from low efficiency and accuracy, because RNA is known to be more labile and less stable than DNA. Other techniques include measuring tissue-specific nucleases, which may require the use of an invasive technique for clinical evaluation (e.g., invasive biopsy or amniocentesis or chorionic villus sampling).

Accordingly, there is a need for a more robust, efficient, reproducible, and effective technique that can non-invasively determine nuclease expression levels or other related values, e.g., related to an abnormality in a subject.

### BRIEF SUMMARY

The present disclosure describes techniques for using nuclease expression in tissues that influences cell-free DNA end signatures/motifs. As examples, an end signature corresponding to a particular nuclease can be in the form of a DNA ending sequence (e.g., sequence end signature) or a specified length of overhang between the DNA strands (e.g., jagged end signature, as may be measured as a jagged end index). In several aspects, the relationship between tissue nuclease expression level and cell-free DNA end signatures can be used to differentiate abnormal and normal tissues, differentiate tissue types (e.g., hematopoietic vs non-hematopoietic, fetal vs maternal), and determine fractional concentration of clinically relevant DNA or a characteristic of a target tissue type.

In another aspect, the biological sample can be enriched for cell-free DNA molecules having a specified length or lengths of jagged ends. The sequence reads from the enriched cell-free DNA molecules can be analyzed to identify a subset of sequence reads that corresponds to a DNA end signature associated with a particular nuclease expression. The subset of sequence reads can be used to determine a parameter to identify a characteristic of the biological sample (e.g., hematopoietic, non-hematopoietic, tumoral, non-tumoral, maternal, fetal, etc).

In yet another aspect, present disclosure describes techniques for analyzing cell-free DNA end signatures of viruses. In one example, relative frequencies of a set of sequence motifs can be identified from the set of the sequence reads obtained from cell-free viral DNA, and the determined relative frequencies can be used to determine a pathology (e.g., nasopharyngeal carcinoma) in a subject. In one embodiment, the pathology can be associated with a virus infection (e.g., Epstein-Barr virus and nasopharyngeal carcinoma, lymphoma or gastric carcinoma; or human papillomavirus and cervical cancer, or hepatitis B virus and hepatocellular carcinoma). In another example, a jaggedness index value determined based on measured properties of cell-free viral DNA can also be used to determine a condition of the subject.

These and other embodiments of the disclosure are described in detail below. For example, other embodiments are directed to systems, devices, and computer readable media associated with methods described herein.

Reference to the remaining portions of the specification, including the drawings and claims, will realize other features and advantages of the present disclosure. Further features and advantages of the present disclosure, as well as the structure and operation of various embodiments of the present disclosure, are described in detail below with respect to the accompanying drawings. In the drawings, like reference numbers can indicate identical or functionally similar elements.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows examples for end motifs according to some embodiments.

FIG. 2 illustrates one example showing how the degree of overhangs of cell-free DNA molecules according to some embodiments.

FIG. 3 shows examples of nuclease-cutting end signatures according to some embodiments.

FIG. 4 shows examples of expression profiles corresponding to different nucleases across different tissues, according to some embodiment.

FIG. 5 shows a model of cfDNA generation and digestion with cutting preferences shown for nucleases DFFB, DNASE1, and DNASE1L3 according to some embodiments.

FIG. 6 shows an example distribution of cell-free DNA molecules with certain end signatures for determining the physiological or pathological state of a tissue, according to some embodiments.

FIGS. 7A and 7B show boxplots that illustrate motif diversity scores and DNASE1L3/DFFB-cutting signature ratios across different tissue groups, according to some embodiments.

FIG. 8 shows receiver operating characteristic (ROC) curves for assessing different parameters for detection of end signatures, according to some embodiments.

FIG. 9 shows a three-dimensional scatter plot of DNASE1L3-, DFFB- and DNASE1-cutting signatures in accordance with some embodiments.

FIG. 10 shows ROC curves depicting performance levels of using logistic regression to determine DNASE1L3-, DFFB-, and DNASE1-cutting signatures, according to some embodiments.

FIG. 11 shows a boxplot depicting the ratio of two plasma end motifs (ACGA/CCCG) according to some embodiments.

FIG. 12 shows a boxplot depicting the ratio of two plasma end motifs (ACGA/CCCG) between wildtype mice and DNASE1L3-deleted mice, according to some embodiments.

FIG. 13 shows percentage of plasma DNA fragments carrying AAAT end motif between wildtype (DFFB^{+/+}) and DFFB deletion mice (DFFB^{-/-}), according to some embodiments.

FIG. 14 shows a percentage of plasma DNA fragments carrying AAAT end motif between human subjects with and without hepatocellular carcinoma (HCC), according to some embodiments.

FIG. 15A shows a boxplot of DNASE1L3/DFFB-cutting signature ratio values across human healthy control subjects (CTR), subjects with chronic hepatitis B infection (HBV) and subjects with HCC, and FIG. 15B shows ROC curves between patients with and without HCC using DNASE1L3/DFFB-cutting signature ratio (densely dashed line), percentage of fragments with end motif CCCA (CCCA, loosely dashed line) and motif diversity score (MDS, solid line), in accordance with some embodiments.

FIG. 16 shows a boxplot of DNASE1/DNASE1L3-cutting signature ratio values across control subjects (e.g., pregnant women without preeclampsia) and pregnant subjects with preeclampsia.

FIG. 17 is a flowchart classifying a level of abnormality in a biological sample based on sequence end signatures, according to some embodiments.

FIGS. 18A and 18B show examples of differentiating maternal and fetal DNA molecules using motif diversity score and DNASE1L3/DFFB-cutting signature ratio, according to some embodiments.

FIG. 19 shows a boxplot of the ratio of two plasma end motifs (CGAA/AAAA) for differentiating fetal and maternal DNA molecules, in accordance with some embodiments.

FIG. 20 shows ROC curves for MDS, CCCA % and DNASE1L3/DFFB-cutting signature ratio in differentiating maternal and fetal DNA molecules, according to some embodiments.

FIGS. 21A and 21B show examples of differentiating liver-derived DNA molecules and DNA molecules of hematopoietic origin using motif diversity score and DNASE1L3/DFFB-cutting signature ratio, according to some embodiments.

FIG. 22 shows ROC curves for MDS, CCCA % and DNASE1L3/DFFB-cutting signature ratio in differentiating liver-derived DNA molecules and DNA molecules of hematopoietic origin, according to some embodiments.

FIG. 23 is a flowchart illustrating a method for estimating a fractional concentration of clinically-relevant DNA molecules in a biological sample, based on sequence end signatures in accordance with some embodiments.

FIGS. 24A and 24B show boxplots of Deoxyribonuclease 1-like 3 expression levels across different gestational ages of human placenta tissues (A, DNASE1L3) and murine placenta tissues (B, Dnase113), according to some embodiments.

FIG. 25 shows a boxplot of DNASE1L3/DFFB-cutting signature ratios across different gestational ages according to some embodiments.

FIG. 26 is a flowchart illustrating a method of determining a characteristic of a target tissue type based on sequence end signatures, according to some embodiments.

FIG. 27 shows a set of graphs that show jaggedness of plasma DNA between wild-type mice and mice with DNASE1L3 deletion.

FIG. 28. shows a box plot that identifies jaggedness of plasma DNA (JI-M) between Dnase1^{-/-} mice and WT mice.

FIG. 29 shows a set of graphs that identify jaggedness of plasma DNA between WT and DFFB^{-/-} mice.

FIGS. 30A and 30B shows comparisons of jaggedness index values between fetal-specific and shared DNA molecules, according to some embodiments.

FIG. 31A shows gene expression of DNASE1 in placental tissues and white blood cells, FIG. 31B shows a boxplot of unmethylated-jaggedness index (JI-U) values between fetal-specific and shared fragments without size selection, and FIG. 31C shows a boxplot of JI-U values between fetal-specific and shared fragments within a size range of 130 to 160 bp, according to some embodiments.

FIG. 32 shows a graph that identifies a cumulative difference in JI-M values between plasma DNA molecules carrying mutant (tumoral DNA) and wild-type alleles (mainly non-tumoral DNA) in a subject with HCC.

FIG. 33 is a flowchart illustrating a method of determining a fraction of clinically-relevant DNA molecules based on jaggedness index values according to some embodiments.

FIG. 34 shows a boxplot of jaggedness index values of plasma DNA in mice across different genotypes including wildtype, DNASE1^{-/-} and DNASE1L3^{-/-}, according to some embodiments.

FIG. 35A shows a boxplot of DNASE1 gene expression in normal liver tissues and liver cancer tissues, FIG. 35B shows a boxplot of JI-U values between patients without and with HCC, and FIG. 35C shows ROC curves for comparing performance between JI-U values deduced by fragments with and without size selection, according to some embodiments.

FIG. 36 is a flowchart illustrating a method of classifying a level of abnormality of a tissue based on jaggedness index values, according to some embodiments.

FIG. 37 shows a graph identifying the distribution of jagged ends in DNA molecules in human subjects with different genotypes of DNASE1L3 associated variants.

FIG. 38 shows a box plot that identify gene expression level of DNASE1L3 in peripheral blood mononuclear cells between control subjects and patients with SLE.

FIG. 39 shows a set of graphs that identify jaggedness of plasma DNA (JI-U) for control samples, and samples with inactive SLE and active SLE.

FIG. 40 shows receiver operating characteristic (ROC) curves that identify performance of jaggedness index values and size ratio methods for differentiating control subjects and SLE subjects.

FIG. 41 shows a graph that identifies JI-M values across different fragment sizes between 0-hour heparin incubation and 6-hour heparin incubation from wildtype mice.

FIG. 42 shows a graph that identifies JI-M values across different fragment sizes between 0-hour incubation and 6-hour incubation with heparin for DNASE1^{-/-} mice.

FIG. 43 shows a flowchart illustrating a method for detecting a genetic disorder for a gene associated with a nuclease using biological samples including cell-free DNA according to embodiments of the present disclosure.

FIG. 44 shows a flowchart illustrating a method for detecting a genetic disorder for a gene associated with a nuclease using a biological sample including cell-free DNA according to embodiments of the present disclosure.

FIG. 45 shows protocols identifying jaggedness of annealed dsDNA treated with or without ExoT.

FIG. 46 is a flowchart illustrating a method for monitoring activity of a nuclease using a biological sample including cell-free DNA according to embodiments of the present disclosure.

FIGS. 47A and 47B show example graphs depicting the relationship between GC % and jagged end length according to some embodiments.

FIG. 48 shows a boxplot of the percentage of fragments carrying CCGT end motif according to some embodiments.

FIG. 49 shows a classification power analysis for differentiating the maternal and fetal DNA fragments using jagged end index (JI-U), end motif (CCGT), and combined end motif and jagged end analysis according to some embodiments.

FIG. 50 shows a scatter plot between the predicted fetal DNA fractions and actual fetal DNA fractions in plasma DNA samples of pregnant women, according to some embodiments.

FIG. 51 is a scatter plot between the predicted tumor DNA fractions and actual tumor DNA fraction in patients with HCC, according to some embodiments.

FIG. 52 is a flowchart illustrating a method of determining a characteristic of a biological sample based on end signatures derived from cell-free DNA molecules having jagged ends, according to some embodiments.

FIG. 53 illustrates an example of a method using jagged end specific hybridization based targeted capture for enriching a certain number of ends of interest, in accordance with some embodiments.

FIG. 54 illustrates an example of a method using jagged end specific adaptor ligation based amplicon sequencing for enriching a certain number of ends of interest, in accordance with some embodiments.

FIG. 55 illustrates an example of a method using droplet PCR to determine a certain number of jagged ends of interest according to some embodiments.

FIG. 56 shows a boxplot of expression levels of DNASE1L3 between non-tumoral nasopharyngeal epithelial tissues and NPC tissues, according to some embodiments.

FIG. 57A shows a boxplot of DNASE1L3-associated end motif CCCA across different subjects with varying stages of nasopharyngeal carcinoma, and FIG. 57B shows an ROC curve depicting performance levels of end motif CCCA in differentiating EBV DNA positive subjects with and without NPC, according to some embodiments.

FIG. 58 shows a boxplot of motif diversity scores across different subjects with varying stages of nasopharyngeal carcinoma according to some embodiments.

FIG. 59 shows ROC curves for assessing performance levels of combined MDS and size analysis according to some embodiments.

FIG. 60 shows a heatmap of 256 end motifs deduced from plasma EBV DNA fragments across patients with nasopharyngeal carcinoma (NPC) and patients with transiently or persistently positive EBV DNA but without NPC, according to some embodiments.

FIG. 61 shows a heatmap that identifies end motifs of plasma EBV DNA which were preferentially present in non-NPC subjects with positive EBV DNA according to some embodiments.

FIG. 62 is a flowchart illustrating a method of analyzing a biological sample with cell-free viral DNA molecules to determine a level of pathology in a subject from which the biological sample is obtained, in accordance to some embodiments.

FIGS. 63A and 63B show boxplots of jaggedness index values deduced from unmethylated signals across different subjects according to some embodiments.

FIG. 64 shows a boxplot of DNASE1 expression levels between NPC tissues and non-tumoral nasopharyngeal epithelial tissues according to some embodiments.

FIG. 65 is a flowchart illustrating a method of analyzing jagged ends of cell-free viral DNA molecules in a biological sample in accordance with some embodiments.

FIG. 66 illustrates a measurement system according to an embodiment of the present invention.

FIG. 67 illustrates example subsystems that implement a measurement system according to an embodiment of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human (or other animal), such as a pregnant woman, a person with cancer, or a person suspected of having cancer, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. As part of an analysis of a biological sample, at least 1,000 cell-free DNA molecules can be analyzed. As other examples, at least 10,000 or 50,000 or 100,000 or 500,000 or 1,000,000 or 5,000,000 cell-free DNA molecules, or more, can be analyzed.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

A “sequence read” refers to a string of nucleotides sequenced from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

A “cutting site” can refer to a location that nucleic acid, e.g., DNA, was cut by a nuclease, thereby resulting in a nucleic acid, e.g., DNA, fragment.

A sequence read can include an “ending sequence” associated with an end of a fragment. The ending sequence can correspond to the outermost N bases of the fragment, e.g., 2-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” of “sequence end signature” may refer to a short, recurring pattern of bases in nucleic acid fragments (e.g., cell-free DNA fragments). A sequence motif can occur at an end of a fragment, and thus be part of or include an ending sequence. An “end motif” can refer to a sequence motif for an ending sequence that preferentially occurs at ends of nucleic acid, e.g., DNA, fragments, potentially for a particular type of tissue. An end motif may also occur just before or just after ends of a fragment, thereby still corresponding to an ending sequence.

The term “jagged end” may refer to sticky ends of nucleic acid (e.g., DNA), overhangs of nucleic acid, or where a double-stranded nucleic acid includes a strand of nucleic acid not hybridized to the other strand of nucleic acid. “Jaggedness index value” is a measure of the extent of a jagged end. The jaggedness index value may be proportional to an average length of one strand that overhangs a second strand in double-stranded nucleic acid. The jaggedness index value of a plurality of nucleic acid molecules may include consideration of blunt ends among the nucleic acid molecules.

In some instances, the jaggedness index value can provide a collective measure that a strand overhangs another strand in a plurality of cell-free DNA molecules. The collective measure of jaggedness can be determined based on an estimated length of overhang in the plurality of cell-free DNA molecules, e.g., an average, median, or other collective measure of individual measurements of each of the cell-free DNA molecules. In some instances, the collective measure of jaggedness is determined for a particular fragment size range (e.g., 130-160 bps, 200-300 bps). In some instances, the collective measure of jaggedness can be determined based on the methylation signal changes proximal to the ends of the plurality of cell-free DNA molecules.

The term “length of overhang” between the DNA strands may refer to a value that can be estimated by comparing the jaggedness (e.g., jaggedness index values) of overall plasma DNA or plasma DNA within a certain fragment size range between reference samples (e.g., normal cells) and differentially-regulated nuclease samples (e.g., tumor cells). In some instances, the length of overhang varies based on a specific DNA fragment size range (e.g., 130-160 bp, 200-300 bp) selected for determining a characteristic of the biological sample.

In some embodiments, the length of overhang in the DNA strands is a categorical value that characterize the length of overhang between two DNA strands. For example, a “long” overhang can include an overhang of a DNA strand that has a size of 5 nt, 6 nt, 7 nt, 8 nt, 10 nt, 15 nt, 20 nt, 30 nt, 40 nt, 50 nt, 100 nt, and greater than 100 nt. A “short” overhang can include an overhang of a DNA strand that has a size of 0 nt, 1 nt, 2 nt, 3 nt, 4 nt, 5 nt. Additionally or alternatively, the specified length of overhang in DNA strands can be estimated based on a percentage of molecules that have a size of overhang that exceeds a particular threshold. For instance, a presence of “long” overhang in plasma DNA could be expressed as the percentage of molecules greater than 5 nt, 6 nt, 7 nt, 8 nt, 10 nt, 15 nt, 20 nt, 30 nt, 40 nt, 50 nt, 100 nt, or their combinations.

An “ending signature” may refer to a sequence motif, a jagged end, or both.

The term “alleles” refers to alternative nucleic acid (e.g., DNA) sequences at the same physical genomic locus, which may or may not result in different phenotypic traits. In any particular diploid organism, with two copies of each chromosome (except the sex chromosomes in a male human subject), the genotype for each gene comprises the pair of alleles present at that locus, which are the same in homozygotes and different in heterozygotes. A population or species of organisms typically include multiple alleles at each locus among various individuals. A genomic locus where more than one allele is found in the population is termed a polymorphic site. Allelic variation at a locus is measurable as the number of alleles (i.e., the degree of polymorphism) present, or the proportion of heterozygotes (i.e., the heterozygosity rate) in the population. As used herein, the term “polymorphism” refers to any inter-individual variation in the human genome, regardless of its frequency. Examples of such variations include, but are not limited to, single nucleotide polymorphism, simple tandem repeat polymorphisms, insertion-deletion polymorphisms, mutations (which may be disease causing) and copy number variations. The term “haplotype” as used herein refers to a combination of alleles at multiple loci that are transmitted together on the same chromosome or chromosomal region. A haplotype may refer to as few as one pair of loci or to a chromosomal region, or to an entire chromosome or chromosome arm.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672).

A “relative frequency” may refer to a proportion (e.g., a percentage, fraction, or concentration). In particular, a relative frequency of a particular end motif (e.g., CCGA) can provide a proportion of cell-free DNA fragments that are associated with the end motif CCGA, e.g., by having an ending sequence of CCGA.

An “aggregate value” may refer to a collective property, namely a value or parameter that describes a property of a dataset with more than one number or measurement, e.g., of relative frequencies of a set of end motifs. Examples include a mean, a median, a sum of relative frequencies, a variation among the relative frequencies (e.g., entropy, standard deviation (SD), the coefficient of variation (CV), interquartile range (IQR) or a certain percentile cutoff (e.g., 95^{th} or 99th percentile) among different relative frequencies), or a difference (e.g., a distance) from a reference pattern of relative frequencies, as may be implemented in clustering.

A “calibration sample” can correspond to a biological sample whose fractional concentration of clinically-relevant nucleic acid (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” and a measured or known characteristic value of a target tissue type or a fractional concentration of the clinically-relevant nucleic acid (e.g., DNA of particular tissue type). The calibration value can be determined from various types of data measured from nucleic acid molecules of a sample, e.g., amounts of end motifs or jaggedness index values. The calibration value corresponds to a parameter that correlates to the desired property, e.g., characteristic value of a target tissue type or a fractional concentration of the clinically-relevant DNA. For example, a calibration value can be determined from relative frequencies (e.g., an aggregate value) of end signatures as determined for a calibration sample, for which the desired property is known. The calibration data points may be defined in a variety of ways, e.g., as discrete points or as a calibration function (also called a calibration curve or calibration surface). The calibration function could be derived from additional mathematical transformation of the calibration data points.

A “separation value” corresponds to a difference or a ratio involving two values, e.g., two fractional contributions or two methylation levels. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (1n) of the two values. A separation value can include a difference and a ratio.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1). As further examples, the levels of classification can correspond to a fractional concentration or a value for a characteristic, e.g., of a sample or of a target tissue type.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics (parameters) can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity). A parameter can be compared to cutoff value, threshold value, reference value, or calibration value to determine a classification Such a process for determining such values can be performed as part of training a machine learning model, e.g., which receives a training vector of a set of one or more parameters. And the comparison of a parameter(s) to any of such values can be accomplished by inputting the parameter(s) into a machine learning model, e.g., that was trained that was trained using the parameter values determined from other subjects, e.g., ones with or without a condition, abnormality, or pathology or ones with a known parameter values (e.g., a calibration value).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer.

A “level of abnormality” can refer to the amount, degree, or severity of abnormality associated with an organism, where the level can be as described above for cancer. An example of abnormality is pathology associated with the organism. Another example of abnormality is a rejection of a transplanted organ. Other example abnormalities can include autoimmune attack (e.g., lupus nephritis damaging the kidney or multiple sclerosis), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of normal.

The term “gestational age” can refer to a measure of the age of a pregnancy which is taken from the beginning of the woman's last menstrual period (LMP), or the corresponding age of the gestation as estimated by a more accurate method if available. Such methods include adding 14 days to a known duration since fertilization (as is possible in in vitro fertilization), or by obstetric ultrasonography.

The term “damage” when describing DNA molecules may refer to DNA nicks, single strands present in double-stranded DNA, overhangs of double-stranded DNA, oxidative DNA modification with oxidized guanines, abasic sites, thymidine dimers, oxidized pyrimidines, blocked 3′ end, or a jagged end.

A “site” (also called a “genomic site”) corresponds to a single site, which may be a single base position or a group of correlated base positions, e.g., a CpG site or larger group of correlated base positions. A “locus” may correspond to a region that includes multiple sites. A locus can include just one site, which would make the locus equivalent to a site in that context.

The “methylation index” or “methylation status” for each genomic site (e.g., a CpG site) can refer to the proportion of nucleic acid fragments (e.g., DNA fragments as determined from sequence reads or probes) showing methylation at the site over the total number of reads covering that site. A “read” can correspond to information (e.g., methylation status at a site) obtained from a nucleic acid fragment. A read can be obtained using reagents (e.g., primers or probes) that preferentially hybridize to nucleic acid fragments of a particular methylation status. Typically, such reagents are applied after treatment with a process that differentially modifies or differentially recognizes nucleic acid molecules depending of their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines.

The “methylation density” of a region can refer to the number of reads at sites within the region showing methylation divided by the total number of reads covering the sites in the region. The sites may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer the number of cytosine sites, “C's”, that are shown to be methylated (for example unconverted after bisulfite conversion) over the total number of analyzed cytosine residues, i.e. including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g., methylation-sensitive restriction enzymes), methylation binding proteins, single molecule sequencing using a platform sensitive to the methylation status (e.g., nanopore sequencing (Schreiber et al. Proc Natl Acad Sci 2013; 110: 18910-18915) and by the Pacific Biosciences single molecule real time analysis (Flusberg et al. Nat Methods 2010; 7: 461-465)).

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and in some versions within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. It is also to be understood that the endpoints of the range provided are included in the range. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described

### DETAILED DESCRIPTION

The present disclosure describes techniques that can use nuclease expression in certain tissue(s) or type(s) of DNA, which influences cell-free DNA end signatures in a cell-free sample (e.g., plasma or serum), to determine properties of the certain tissue(s) or type(s) of DNA via non-invasive measurements of the cell-free sample. In an example of a nuclease being differentially regulated in abnormal cells of a target tissue type relative to normal cells, a measurement of an end signature in cell-free DNA molecules in a sample can be used to determine a level of abnormality in the sample/subject, e.g., a presence of abnormal cells. For example, Deoxyribonuclease 1 Like 3 (DNASE1L3) expression is relatively downregulated in hepatocellular carcinoma (HCC) cells compared with liver tissues in healthy subjects.

The differentially-regulated nuclease can be assessed to identify that it preferentially cuts DNA into DNA molecules that have a particular end signature. In various embodiments, the end signatures corresponding to a particular nuclease can be identified in at least two different forms: (i) a sequence end motif; and (ii) a specified length of overhang between the DNA strands (e.g., jagged end signature). For example, an end signature of an DNASE1L3 expression can be CCCA end motif sequences. As another example, a particular nuclease can favor a larger overhang (or smaller overhang) than is typical (normal) in such cell-free samples.

The end signatures of cell-free DNA molecules can be used to determine different types of parameters based on sequence reads obtained from a biological sample that includes the cell-free DNA molecules. For example, a parameter can be a ratio of amounts between two end motifs (e.g., CCCA/AAAT). In another example, a parameter can be a jaggedness index value that identifies a measure of the extent of a jagged end in the DNA molecules. Based on these parameters, the relationship between tissue nuclease expression level and cell-free DNA end signatures can be used to differentiate abnormal and normal tissues, differentiate tissue types (e.g., hematopoietic vs non-hematopoietic, fetal vs maternal), and determine fractional concentration of clinically relevant DNA or a characteristic of a target tissue type.

In some instances, the biological sample can be enriched for cell-free DNA molecules having a specified length or lengths of jagged ends. Different techniques may be used to enrich cell-free DNA molecules having the specified length of overhang between the first strand and the second strand, including jagged end specific hybridization based targeted capture, jagged end specific adaptor ligation based amplicon sequencing, and digital PCR (e.g., droplet digital PCR). The sequence reads from the enriched cell-free DNA molecules can be analyzed to identify a subset of sequence reads that corresponds to a sequence end signature associated with a particular nuclease.

With or without a jaggedness enrichment, the subset of sequence reads may include an CCCA end motif sequence, which is an end signature associated with DNASE1L3 expression. The subset of sequence reads can be used to determine a parameter (e.g., a ratio between CCCA/AAAT) to identify a characteristic of the biological sample. For example, the determined characteristic can include a particular gestational age or range (e.g., 8 weeks, 9-12 weeks), e.g., when a nuclease is differentially regulated between fetal tissue and maternal tissue. In another example, the determined characteristic can be a size or nutrition status of an organ corresponding a particular tissue type (e.g., liver cells), which is differentially regulated relative to another tissue type (e.g., hematopoietic cells).

The present disclosure also describes techniques for analyzing cell-free DNA end signatures of viruses. A set of the sequence reads aligning to a reference virus genome are determined. For each of the set of sequence reads, a sequence end motif is determined. Based on the sequence end motifs corresponding to the set of sequence reads, relative frequencies of a set of sequence motifs can be identified, for which an aggregate value (e.g., a motif diversity score) can be determined. The aggregate value can be used to determine a pathology (e.g., a cancer such as nasopharyngeal carcinoma) in a subject. In one embodiment, the pathology can be associated with a virus infection (e.g., Epstein-Barr virus and nasopharyngeal carcinoma, lymphoma or gastric carcinoma; or human papillomavirus and cervical cancer, or hepatitis B virus and hepatocellular carcinoma).

In some instances, a jaggedness index value determined based on measured properties of cell-free viral DNA can also be used to determine a condition of the subject. A set of the sequence reads aligning to a reference virus genome can be determined. For each of the set of sequence reads, a property of the first strand and/or the second strand that is proportional to a length of the first strand that overhangs the second strand. Based on the measured properties, the jaggedness index value can be determined. The jaggedness index value can be compared to a reference value to determine the condition of the subject (e.g., HCC, colorectal cancer, leukemia, lung cancer, breast cancer, prostate cancer, throat cancer, etc.).

Certain techniques described herein improve differentiating abnormal and normal tissues, differentiating tissue types (e.g., hematopoietic vs non-hematopoietic, fetal vs maternal), and determining fractional concentration of clinically relevant DNA by leveraging nuclease expression in tissues that influences cell-free DNA end signatures/motifs. In addition, the techniques based on cell-free DNA end signatures can be advantageous over techniques that solely analyze nuclease expression levels. For example, genetic analysis of nuclease expression levels may involve RNA sequencing or other type of RNA analyses (e.g., reverse transcriptase polymerase chain reaction). RNA is known to be more labile and less stable than DNA, due to its susceptibility to hydrolysis. Accordingly, sample collection, preparation and analysis protocols can be more robust, efficient, reproducible and effective for DNA analysis than RNA. Moreover, when short read sequencing is used to analyze circulating RNA, additional metrics are needed to translate fragment count to expression levels because circulating RNA has a wider range of molecular length. One molecule can generate more than one fragment but should be counted as having expressed once only. In view of the above, cell-free DNA end signatures derived from nuclease expression levels can be a more accurate and/or practical indicator for different types of clinical evaluation of a subject.

In addition, tissue-specific nucleases that act locally cannot be easily measured. These nucleases may need to be measured by analyzing the tissue, which may require the use of an invasive technique for clinical evaluation (e.g., invasive biopsy or amniocentesis or chorionic villus sampling). On the other hand, nuclease expression levels can be reflected in cell-free DNA molecules with corresponding end signature that would circulate in plasma. Such signatures can be obtained through analysis of plasma DNA, which is a far less invasive technique compared to nuclease analysis of tissue cells.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### I. Cell-Free DNA End Motifs

An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

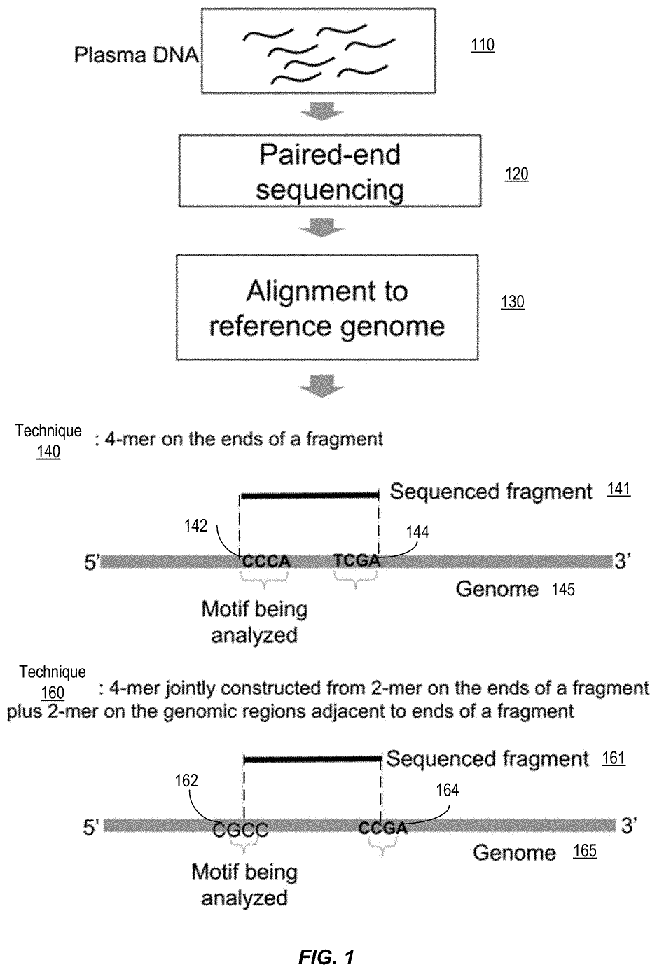

FIG. 1 shows examples for end motifs according to embodiments of the present disclosure. FIG. 1 depicts two ways to define 4-mer end motifs to be analyzed. In technique 140, the 4-mer end motifs are directly constructed from the first 4-bp sequence on each end of a plasma DNA molecule. For example, the first 4 nucleotides or the last 4 nucleotides of a sequenced fragment could be used. In technique 160, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, 7-mer end motifs.

As shown in FIG. 1, cell-free DNA fragments 110 are obtained, e.g., using a purification process on a blood sample, such as by centrifuging. Besides plasma DNA fragments, other types of cell-free DNA molecules can be used, e.g., from serum, urine, saliva, and other mentions herein. In one embodiment, the DNA fragments may be blunt-ended.

At block 120, the DNA fragments are subjected to paired-end sequencing. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment.

At block 130, the sequence reads can be aligned to a reference genome. This alignment is to illustrate different ways to define a sequence motif, and may not be used in some embodiments. The alignment procedure can be performed using various software packages, such as BLAST, FASTA, Bowtie, BWA, BFAST, SHRiMP, SSAHA2, NovoAlign and SOAP.

Technique 140 shows a sequence read of a sequenced fragment 141, with an alignment to a genome 145. With the 5′ end viewed as the start, a first end motif 142 (CCCA) is at the start of sequenced fragment 141. A second end motif 144 (TCGA) is at the tail of the sequenced fragment 141. When analyzing the end predominance of a cell-free DNA (cfDNA) fragments (e.g., plasma DNA), this sequence read would contribute to a C-end count for the 5′ end. Such end motifs might, in one embodiment, occur when an enzyme recognizes CCCA and then makes a cut just before the first C. If that is the case, CCCA will preferentially be at the end of the plasma DNA fragment. For TCGA, an enzyme might recognize it, and then make a cut after the A.

Technique 160 shows a sequence read of a sequenced fragment 161, with an alignment to a genome 165. With the 5′ end viewed as the start, a first end motif 162 (CGCC) has a first portion (CG) that occurs just before the start of sequenced fragment 161 and a second portion (CC) that is part of the ending sequence for the start of sequenced fragment 161. A second end motif 164 (CCGA) has a first portion (GA) that occurs just after the tail of sequenced fragment 161 and a second portion (CC) that is part of the ending sequence for the tail of sequenced fragment 161. Such end motifs might, in one embodiment, occur when an enzyme recognizes CGCC and then makes a cut just before the G and the C. If that is the case, CC will preferentially be at the end of the plasma DNA fragment with CG occurring just before it, thereby providing an end motif of CGCC. As for the second end motif 164 (CCGA), an enzyme can cut between C and G. If that is the case, CC will preferentially be at the end of the plasma DNA fragment. For technique 160, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end signature, the higher the specificity of the motif because the probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

As the ending sequence is used to align the sequence read to the reference genome, any sequence motif determined from the ending sequence or just before/after is still determined from the ending sequence. Thus, technique 160 makes an association of an ending sequence to other bases, where the reference is used as a mechanism to make that association. A difference between techniques 140 and 160 would be to which two end motif a particular DNA fragment is assigned, which affects the particular values for the relative frequencies. But, the overall result (e.g., fractional concentration of clinically-relevant DNA, classification of a level of pathology, etc.) would not be affected by how the a DNA fragment is assigned to an end motif, as long as a consistent technique is used for the training data as used in production.

The counted numbers of DNA fragments having an ending sequence corresponding to a particular end motif may be counted (e.g., stored in an array in memory) to determine relative frequencies. As described in more detail below, a relative frequency of end motifs for cell-free DNA fragments can be analyzed. Differences in relative frequencies of end motifs have been detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

### II. Jagged Ends in Cell-Free DNA

Cell-free DNA ends would be classified into two forms according to modalities of ends. One form of cell-free DNA would be present in blood circulation with blunt ends and the other would carry sticky ends. A sticky end is an end of a double-stranded DNA that has at least one outermost nucleotide not hybridized to the other strand. Sticky ends are also called overhangs or jagged ends. Without intending to be bound by any particular theory, it is thought that the jagged ends may be related to how cell-free DNA is cut, broken, or degraded into fragments. For example, DNA may fragment in stages, and the size of the jagged end may reflect the stage of fragmentation. The number of jagged ends and/or the size of an overhang in a jagged end may be used to analyze a biological sample with cell-free DNA and provide information of about the sample and/or the individual from which the sample is obtained.

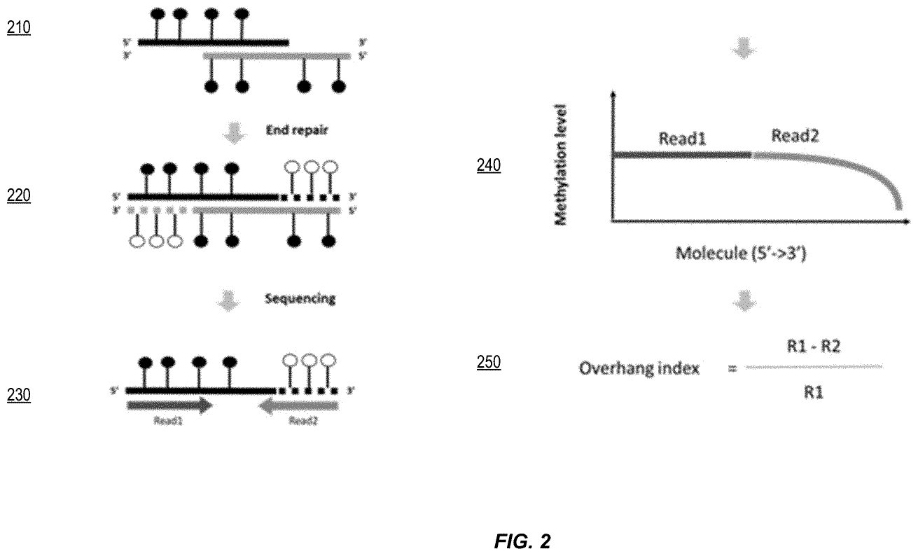

FIG. 2 illustrates one example showing how the degree of overhangs of cell-free DNA molecules (i.e. overhang index) can be deduced. Diagrams 210, 220, 230 illustrate examples of cell-free DNA molecules, in which filled lollipops represent methylated CpG sites and unfilled lollipops represent unmethylated CpG sites. In diagrams 220 and 230, the dashed lines represent newly filled-up nucleotides that include unfilled lollipops. In diagram 230, a red arrow pointing from left-to-right represents a first read (read 1) in sequencing results and a cyan arrow pointing from right-to-left represents a second read (read 2). Further, graph 240 shows methylation level in read 1 and read 2 from 5′ to 3′. Equation 250 shows an equation determining an overhang index of the cell-free DNA molecule, in which R1 represents the methylation level of read 1 and R2 represents the methylation level of read 2.

The following process illustrates an example of using jaggedness index values to analyze a biological sample. The biological sample may be obtained from an individual. The biological sample may include a plurality of nucleic acid molecules, which are cell-free. Each nucleic acid molecule of the plurality of nucleic acid molecules may be double-stranded with a first strand having a first portion and a second strand. The first portion of the first strand of at least some of the plurality of nucleic acid molecules may overhang the second strand, may not be hybridized to the second strand, and may be at a first end of the first strand. The first end may be a 3′ end or a 5′ end. Analysis of jagged ends in plasma DNA molecules can be performed using various approaches described in US Patent Publication No. 2020/0056245/A1, filed Jul. 23, 2019, the entire contents of which are incorporated herein by reference in its entirety and for all purposes.

The process may include measuring a property of a first strand and/or a second strand that is proportional to a length of the first strand that overhangs the second strand. The property may be measured for each nucleic acid of a plurality of nucleic acids. The property may be measured by any technique described herein.

The property may be a methylation status at one or more sites at end portions of the first and/or second strands of each of the plurality of nucleic acid molecules. The jaggedness index value may include a methylation level over the plurality of nucleic acid molecules at one or more sites of end portions of the first and/or second strands.

In some embodiments, the process includes measuring sizes of nucleic acid molecules. The plurality of nucleic acid molecules may have sizes within a specified range. The specified range may be from 140 to 160 bp, any range less than the entire range of sizes present in the biological sample, or any range described herein. The size range may be based on the size of the shorter strand or the longer strand. The size range may be based on the outermost nucleotides of molecules after end repair. If the 5′ end protrudes, then 5′ to 3′ polymerase mediated elongation will occur and the size may be the longer strand. If the 3′ end protrudes, without a DNA polymerase with a 3′ to 5′ synthesis function, the 3′ protruded single-strand may be trimmed and the size may then be the shorter strand.

In embodiments, the process may include analyzing nucleic acid molecules to produce reads. The reads may be aligned to a reference genome. The plurality of nucleic acid molecules may be reads within a certain distance range relative to a transcription start site.

The process may include determining the jaggedness index value using the measured properties of the plurality of nucleic acid molecules.

If the first plurality of nucleic acid molecules are in a specified size range, methods may include measuring the property of each nucleic acid molecule of a second plurality of nucleic acid molecules. The second plurality of nucleic acid molecules may have sizes with a second specified size range. Determining the jaggedness index value may include calculating a ratio using the measured properties of the first plurality of nucleic acid molecules and the measured properties of the second plurality of nucleic acid molecules. The jaggedness index value may include the jagged end ratio or the overhang index ratio described herein.

The process may compare the jaggedness index value to a reference value. The reference value or the comparison may be determined using machine learning with training data sets. The comparison may be used to determine different information regarding the biological sample or the individual.

The process may include determining a level of a condition of an individual based on the comparison. The condition may include a disease, a disorder, or a pregnancy. The condition may be cancer, an auto-immune disease, a pregnancy-related condition, or any condition described herein. As examples, cancer may include hepatocellular carcinoma (HCC), colorectal cancer (CRC), leukemia, lung cancer, breast cancer, prostate cancer or throat cancer. The auto-immune disease may include systemic lupus erythematosus (SLE). Various data below provides examples for determined a level of a condition.

In some instances, the reference value is determined using one or more reference samples of subjects that have the condition. As another example, the reference value is determined using one or more reference samples of subjects that do not have the condition. Multiple reference values can be determined from the reference samples, potentially with the different reference values distinguishing between different levels of the condition.

The process may include determining a fraction of clinically-relevant DNA in a biological sample based on the comparison. Clinically-relevant DNA may include fetal DNA, tumor-derived DNA, or transplant DNA. The reference value may be obtained using nucleic acid molecules from one or more reference subjects having a known fraction of clinically-relevant DNA. Methods for determining the fraction of clinically-relevant DNA may include treating the plurality of nucleic acid molecules by a protocol before measuring the property of the first strand and/or the second strand. The nucleic acid molecules from one or more reference subjects may be treated by the same protocol as the plurality of nucleic acid molecules having the property measured.

Calibration data points can include a measured jaggedness index value and a measured/known fraction of the clinically-relevant DNA. The measured jaggedness index value for any sample whose fraction is measured via another technique (e.g., using a tissue-specific allele) can be correspond to a reference value. As another example, a calibration curve (function) can be fit to the calibration data points, and the reference value can correspond to a point on the calibration curve. Thus, a measured jaggedness index value of a new sample can be input into the calibration function, which can output the faction of the clinically-relevant DNA.

### III. Differential Regulation of Nucleases

Cell-free DNA (cfDNA) is a powerful non-invasive biomarker for cancer and prenatal testing and circulates in plasma as short fragments. To elucidate the biology of cfDNA fragmentation, we explored the roles of DNASE1, DNASE1L3, and DNA fragmentation factor subunit beta (DFFB) with mice deficient in each of these nucleases. By analyzing the ends of cfDNA fragments in each type of nuclease-deficient mice with those in wildtype mice, we have shown that each nuclease has a specific cutting preference that reveals the stepwise process of cfDNA fragmentation. We demonstrate that the DNA fragmentation first begins intracellularly with DFFB, intracellular DNASE1L3, and other nucleases. Then, cfDNA fragmentation continues extracellularly with circulating DNASE1L3 and DNASE1. With the use of heparin to disrupt the nucleosomal structure, we also showed that the 10 bp periodicity originated from the cutting of DNA within an intact nucleosomal structure. Altogether, this work establishes a model of cfDNA fragmentation.

Cell-free DNA (cfDNA) molecules are nonrandomly fragmented. It was reported that cfDNA fragmentation patterns were associated with the nucleosome structures (Sun et al. Proc Natl Acad Sci USA. 2018; 115:E5106; Snyder et al. Cell. 2016; 164:57-68). The nonrandomness of cfDNA molecules is also reflected by the characteristic size profile, showing a modal frequency at approximately 166 bp, with smaller molecules forming a series of peaks that exhibit a 10 bp periodicity (Lo et al. Sci Transl Med. 2010; 2:61ra91). Recently, a subset of genomic locations were found to be preferentially cut during the generation of plasma DNA molecules (Chan et al. Proc Natl Acad Sci USA. 2016; 113:E8159-E8168; Jiang et al. Proc Natl Acad Sci USA. 2018; 115:E10925-E10933). For instance, a number of genomic sites would be enriched for plasma DNA fragment ends originating from liver tissues (Jiang et al. Proc Natl Acad Sci USA. 2018; 115:E10925-E10933). These data at the time suggested that plasma DNA or cell-free DNA may preferentially fragment at certain genomic locations, namely specific genomic coordinates of the genome. Using mouse models with gene knockouts, we showed that nucleases contribute to plasma DNA fragmentation. We further showed that different nucleases are associated with plasma DNA or cell-free DNA molecules with characteristic end motifs or signatures (Serpas et al. Proc Natl Acad Sci USA. 2019; 116:641-649; Han et al. Am J Hum Genet. 2020; 106:202-14). In other words, other than fragmenting at certain genome locations, these observations suggest that the sequence context of the DNA may influence if it would be a preferred substrate for processing by certain nucleases or not. Here we develop approaches to utilize cell-free DNA end motifs associated with the various nucleases as biomarkers. We show that nuclease enzyme activities would vary across different tissues and change according to different pathophysiological states such as cancer, pregnancy and organ transplantation. The selective analysis of the plasma DNA fragmentation signatures associated with the relevant nucleases that would be aberrant in a particular disease state could be used for detecting and monitoring such a disease.

The relevant nucleases could be defined as those with changes in expression (upregulation or downregulation) according to different pathophysiological conditions across different tissues. Differential regulation of nucleases is measured using approaches described in U.S. Application No. 62/949,867, filed Dec. 18, 2019, and U.S. Application No. 62/958,651, filed Jan. 8, 2020, the entire contents of which are incorporated herein by reference in its entirety and for all purposes. When these tissues release DNA into the circulation, the relative abundances of plasma DNA molecules carrying particular end signatures would change as a result of the altered expression level of the associated nuclease. In one embodiment, the formats of such end signatures could include but not limited to end motifs and jagged ends. End motifs in plasma DNA molecules are measured using approaches described in US Patent Publication No. 2020/0199656 A1, filed Dec. 19, 2019, the entire contents of which are incorporated herein by reference in its entirety and for all purposes. Jagged ends in plasma DNA molecules are measured using approaches described in US Patent Publication No. 2020/0056245/A1, filed Jul. 23, 2019, the entire contents of which are incorporated herein by reference in its entirety and for all purposes.

In some embodiments, a relationship between differential regulation of a nuclease and a condition of a target tissue type (e.g., cancer) can be predicted based on an amount of cell-free DNA molecules having a particular end signature in samples from a subject with the condition for the target tissue, given knowledge about an association of a nuclease with the particular end signature. For example, for a sample from a subject with the condition, a high/low amount of the particular end signature can indicate differential regulation of the nuclease occurs in subject having the condition in the target tissue type.

In other embodiments, an end signature related to a nuclease can be predicted based on an amount of cell-free DNA molecules having a particular end signature. For example, sequence reads obtained from tissue with a differentially regulated nuclease can be used to identify one or more sets of sequence reads having ending sequences corresponding to a respective end signatures. As another example, a high/low amount of a particular end signature in a cell-free sample of a subject known to have a condition for target tissue where the nuclease is differentially regulated.

A. Differential Regulations of Nuclease Between Abnormal and Normal Cells

Across various tissue types (e.g., a liver), a particular nuclease can be differentially regulated in abnormal cells relative to normal cells. This could be attributed to gene mutations of the abnormal cells that result in an increased or decreased expression of such nuclease. For example, DNASE1L3 expression in HCC cells is likely to be downregulated relative to DNASE1L3 expression in normal cells. These differences in nuclease expression between abnormal and normal cells can be used to predict whether a biological sample of a subject includes abnormal cells based on its corresponding nuclease expression.

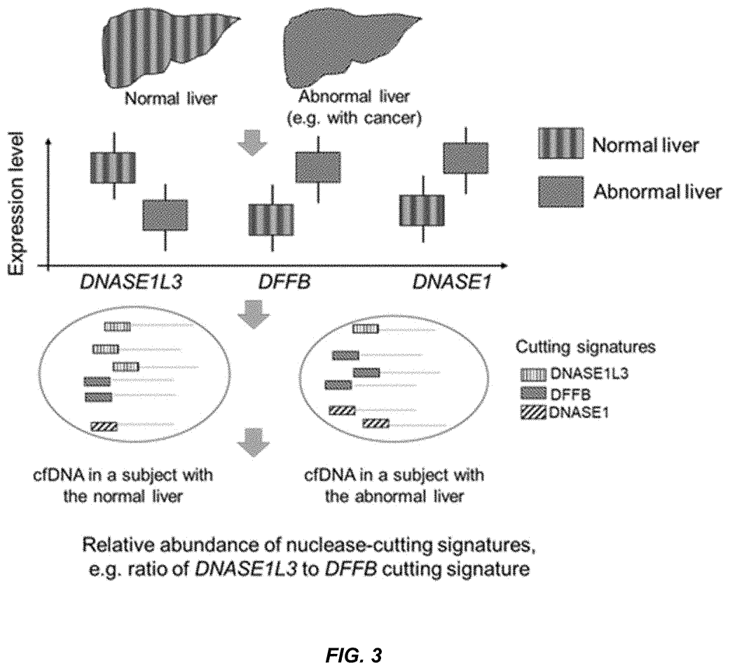

FIG. 3 shows examples of nuclease-cutting end signatures according to some embodiments. Plasma DNA fragmentation process was found to be associated with nuclease cutting in a mouse model (Serpas et al. Proc Natl Acad Sci USA. 2019; 116:641-649; Han et al. Am J Hum Genet. 2020; 106:202-14). We hypothesize that the gene expression of one or more nucleases would be altered in certain pathophysiological states such as cancer (FIG. 3). For example, DNASE1L3Deoxyribonuclease 1 Like 3 (DNASE1L3) expression is relatively downregulated, DFFBDNA Fragmentation Factor Subunit Beta (DFFB) and DNASE1Deoxyribonuclease 1 (DNASE1) expression are relatively upregulated in HCC tissues, compared with liver tissues in healthy subjects. Therefore, the relative activities of nucleases functioning in liver tissues or nucleases entering the blood circulation would be aberrant, leading to the altered abundance of nuclease-cleaved end signatures in plasma DNA.

In one embodiment, the effect in DNA fragmentation caused by nucleases functioning in a local organ/tissue would be defined as a local effect (e.g., due to abnormality in a cell causing differential regulation), while the effect in DNA fragmentation caused by nucleases circulating in blood circulation would be defined as a systemic effect. To specifically analyze the nuclease-related cutting signatures, referred to as nuclease-cutting end signatures, would improve the signal-to-noise ratio, thus improving the performance in differentiating the patients with and without diseases (e.g., cancer). In one embodiment, as shown in FIG. 3, we could use the ratio of two nuclease-cutting signatures (i.e. nuclease-cutting signature ratio) in the plasma DNA pool for which one corresponds to the upregulated nuclease (DNASE1L3) and the other corresponds to downregulated nuclear (DFFB). In one embodiment, one could use other statistical and/or mathematical calculations to utilize one or more nuclease-cutting signatures, including but not limited to, relative/absolute deviations, relative/absolute percentage increases, relative/absolute percentage decreases, linear/non-linear combinations of multiple ratios or deviations, etc. In another embodiment, the nucleases would include, but not limited to, TREX1 (Three Prime Repair Exonuclease 1), AEN (Apoptosis Enhancing Nuclease), EXO1 (Exonuclease 1), DNASE2 (Deoxyribonuclease 2), ENDOG (Endonuclease G), APEX1 (Apurinic/Apyrimidinic Endodeoxyribonuclease 1), FEN1 (Flap Structure-Specific Endonuclease 1), DNASE1L1 (Deoxyribonuclease 1 Like 1), DNASE1L2 (Deoxyribonuclease 1 Like 2) and EXOG (Exo/Endonuclease G).

For illustrative purposes, we use scenarios with liver with or without cancers as examples. The normal liver has a higher expression of DNASE1L3 than DNASE1 and DFFB. Those nucleases would function inside the liver and would promote DNA fragmentation (referred to as the local effect of the nucleases). On the other hand, such nucleases would be passively or actively released into circulation and play role in DNA fragmentation in blood circulation (referred to as systemic effect of the nucleases). As a result, the plasma sample from a subject with a normal liver would show more plasma DNA molecules with end signatures related to DNASE1L3 than those associated with DFFB and DNASE1. However, in certain clinical scenarios, e.g., in a liver with a HCC, the expression levels of different nucleases in the HCC-affected liver would be aberrant. For example, the downregulation of the DNASE1L3 gene expression and upregulation of the DNASE1 and DFFB gene expression occur in a liver with a HCC. Therefore, the DNASE1L3-associated end signatures would be relatively decreased in patients with cancer, while DNASE1-associated and DFFB-associated end signatures would be relatively increased in patients with cancer, compared with those without cancer. The approaches for synergistic profiling of these nucleases associated end signatures are implemented in this disclosure, improving the plasma DNA fragmentomic signals for differentiating patients with and without diseases such as cancer. In one embodiment, the organs having local and systemic effects in DNA cleavage would include, but not limited to, the colon, small intestines, stomach, kidney, bladder, pancreas, brain, lung, salivary gland, dendritic cells, T cells, B cells, thymus, lymph node, monocytes, muscle, heart, placenta, ovary, breast, and testis.

For illustration purposes, we performed paired-end sequencing (75 bp×2 (i.e. paired-end sequencing), Illumina). We have sequenced plasma DNA from healthy controls (n=38), patients with chronic hepatitis B (n=17), patients with HCC (n=34), respectively, with a median number of 38 million paired-end sequencing reads (range: 18-65 million). We also sequenced 10 plasma DNA samples from each of the patient groups with colorectal cancer, lung cancer, nasopharyngeal carcinoma, and head and neck squamous cell carcinoma, with a median number of 42 million paired-end sequencing reads (range: 19-65 million).

On the other hand, we sequenced plasma DNA from wildtype mice (n=9), mice with deletion of the DNASE1 gene (n=3), DNASE1L3 gene (n=13), and DFFB gene (n=5), respectively. The median number of reads was 35 million (range: 16-78 million).

B. Differential Regulations of Nucleases for Different Tissue Types

In addition to differentiating abnormal cells from normal cells, nuclease expression can be used to differentiate tissue types. Nuclease expression detected from a first tissue type can differ from the nuclease expression of a second tissue type. For example, an amount of DNASE1L3 expression detected in liver cells is relatively greater than an amount of DNASE1L3 expression detected in esophageal cells. Further, differences of nuclease expression can also be found in abnormal cells across different tissue types. For example, an amount of DFFB expression detected in abnormal liver cells (e.g., HCC) is relatively less than an amount of DFFB expression detected in abnormal bladder cells (e.g., Bladder Urothelial Carcinoma). These differences in nuclease expression between different tissue types can be used to predict the tissue type from the abnormal cells have originated.

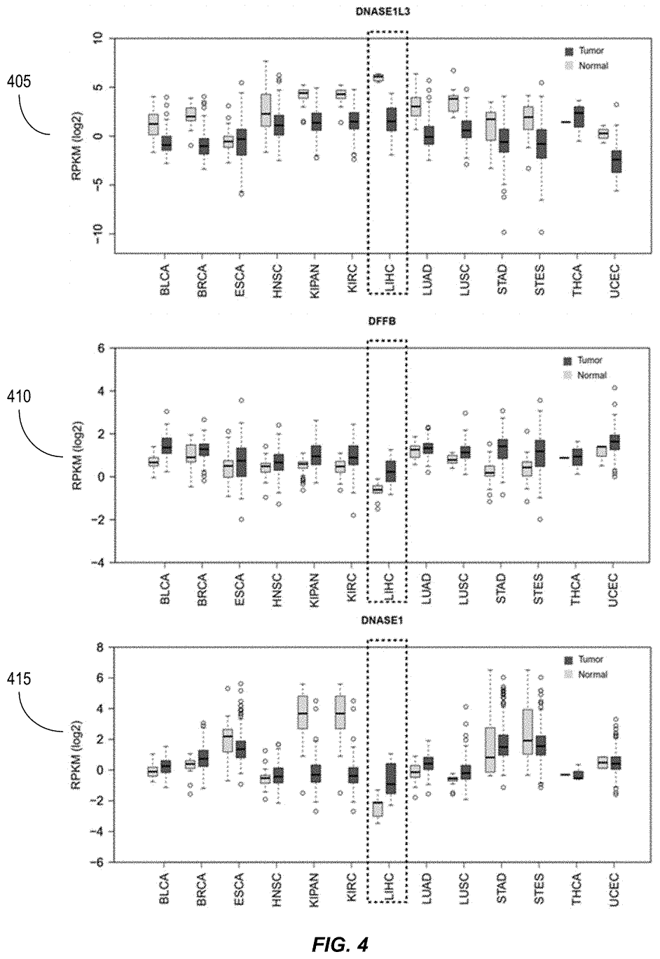

FIG. 4 shows examples of expression profiles corresponding to different nucleases across different tissues, according to some embodiment. For example, a first bar plot 405 shows expression profiles of DNASE1L3 across different tissues, a second bar plot 410 shows expression profiles of DFFB across different tissues, and a third bar plot 415 shows expression profiles of DNASE1 across different tissues. In each of the bar plots 405, 410, and 415, the following acronyms refer to as follows: (1) BLCA—Bladder Urothelial Carcinoma; (2) BRCA—Breast invasive carcinoma; (3) ESCA—Esophageal carcinoma; (4) HNSC—Head and Neck squamous cell carcinoma; (5) KIPAN—Kidney pan cancer including kidney chromophobe, kidney renal clear cell carcinoma, and kidney renal papillary cell carcinoma; (6) KIRC—Kidney renal clear cell carcinoma; (7) LIHC—Liver hepatocellular carcinoma, also referred to as HCC; (8) LUAD—Lung adenocarcinoma; (9) LUSC—Lung squamous cell carcinoma; (10) STAD—Stomach adenocarcinoma; (11) STES—Stomach and Esophageal carcinoma; (12) THCA—Thyroid carcinoma; and (13) UCEC—Uterine Corpus Endometrial Carcinoma.

In addition, RPKM is a normalized gene expression unit deduced from RNA sequencing results, i.e. reads per kilobase per million reads sequenced (Trapnell et al. Nat Biotechnol. 2010; 28:511-5). As shown in FIG. 4, different nucleases have different expression levels across different tissues. For example, DFFB expression in the second bar plot 410 shows difference between HCC and UCEC.

Further, different nucleases have different expression levels between abnormal and normal tissues. For example, the DNASE1L3 expression in the first bar plot 405 showed downregulation in HCC/LIHC tumor tissues (2.85 RPKM) compared with the adjacent non-tumoral tissues (68.18 RPKM) (P value <0.0001, Mann Whitney U test). On the other hand, the DFFB and DNASE1 expressions showed upregulation in HCC/LIHC tumor tissues (1.17 and 0.53 RPKM) compared with the adjacent non-tumoral tissues (0.66 and 0.23 RPKM) (P value <0.0001, Mann Whitney U test).

C. Effects of Differential Regulation of Nucleases on Cell-Free DNA End Motifs

The end motifs could be defined by a number of nucleotides at the ends of cell-free DNA fragments and/or one or several nucleotides close to but not at the fragment ends. In one embodiment, the fragment end refers to the 5′ end. In another embodiment, the fragment end refers to the 3′ end. In yet other embodiments, both the 5′ and 3′ ends are used. The number of nucleotides (nt) at the fragment ends used for analysis would be, for example but not limited to, 1 nucleotide(s) (nt), 2 nt, 3 nt, 4 nt, 5 nt, 6 nt, 7 nt, 8 nt, 9 nt, and 10 nt or above. In one embodiment, nuclease-associated end motif would correspond to sites preferentially cleaved by a nuclease. In another embodiment, nuclease-associated end motifs would correspond to end motifs which are preferentially cut by one or more nucleases. In another embodiment, nuclease-associated end motifs would be defined by those end motifs which are over-represented or under-represented in disease (e.g., cancer) or clinical scenarios (e.g., following transplantation), or in certain physiological states (e.g., pregnancy). In yet another embodiment, nuclease-associated end motifs could be defined by those end motifs which are over-represented or under-represented in nuclease knockout mice or other genetically modified animals.

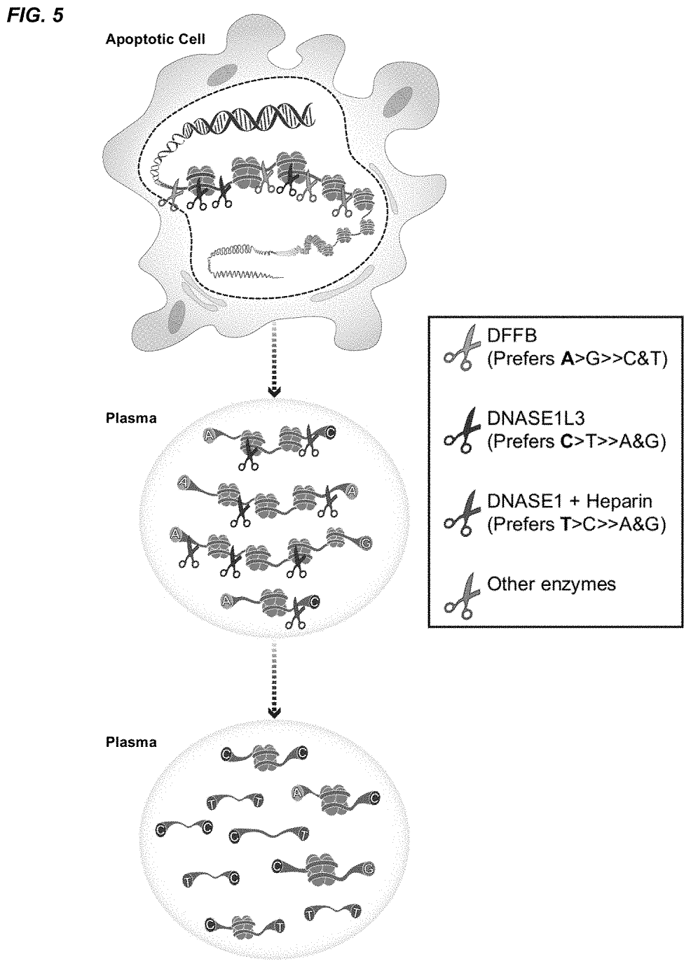

FIG. 5 shows a model of cfDNA generation and digestion with cutting preferences shown for nucleases DFFB, DNASE1, and DNASE1L3 according to some embodiments. DFFB generates fresh cfDNA that is A-end enriched. DNASE1L3 generates the predominantly C-end enriched cfDNA seen in a typical ending profile (also referred to as “profile). DNASE1 with the help of heparin and endogenous proteases can further digest cfDNA into T-end fragments.

FIG. 5 shows an apoptotic cell with DFFB (green scissors) and DNASE1L3 (blue scissors) shown in the cell. The legend shows the preferential order for cutting of the three nucleases for different bases. DFFB is shown acting only in the cell. DNASE1L3 is shown as acting also in plasma. DNASE1 (red scissors) with heparin is shown acting in plasma. The resulting fragments with ending bases are shown, with different colors for the corresponding nucleases. The DNA molecules become shorter after being cut in the cell, and then even shorter after being cut in the plasma.

From this work on cfDNA fragment ends in different mouse models, we can piece together a model outlining the fragmentation process that generated cfDNA. In our analysis of the newly released cfDNA spontaneously created after incubating whole blood in EDTA, we have demonstrated that the fresh longer cfDNA are enriched for A-end fragments. In particular, A< >A, A< >G, and A< >C fragments demonstrate a strong nucleosomal periodicity at −200 bp and 400 bp. When this same experimental model is applied to the whole blood of DFFB-deficient mice, no long A-end fragment enrichment is seen. Thus, we can conclude that DFFB is likely responsible for generating these A-end fragments.

This hypothesis is substantiated by literature published on the DFFB enzyme, which plays a major role in DNA fragmentation during apoptosis (Elmore, S. (2007), Toxicologic pathology 35, 495-516; Larsen, B. D. and Sorensen, C. S. (2017), The FEBS Journal 284, 1160-1170). Enzyme characterization studies have shown that DFFB creates blunt double-strand breaks in open internucleosomal DNA regions with a preference for A and G nucleotides (purines) (Larsen, B. D. and Sorensen, C. S. (2017), The FEBS Journal 284, 1160-1170; Widlak, P., and Garrard, W. T. (2005), Journal of cellular biochemistry 94, 1078-1087; Widlak, P. et al., (2000), The Journal of biological chemistry 275, 8226-8232)). This biology of blunt double-stranded cutting only at internucleosomal linker regions would explain the nucleosomal patterning in A< >A, A< >G, and A< >C fragments.

In this work, we have also demonstrated that typical cfDNA in plasma obtained before incubation predominantly end in C across all fragment sizes; this C-end overrepresentation is consistent in multiple different regions across the genome. Because the typical profile of cfDNA is so different from fresh cfDNA, we can infer that 1) one or more additional nucleases create(s) this profile, 2) this nuclease or these nucleases dominate(s) the cleaving process in typical cfDNA, and 3) this process largely occurs after the generation of fresh A-end fragments.

Since this C-end predominance is lost in DNASE1L3-deficient mice, we believe that one nuclease responsible for creating this C-end fragment overrepresentation is DNASE1L3. While there is no existing enzymatic study that investigates the specific nucleotide cleavage preference of DNASE1L3, DNASE1L3 is known to cleave chromatin with high efficiency to almost undetectable levels without proteolytic help (Napirei, M. et al., (2009), The FEBS Journal 276, 1059-1073); Sisirak, V. et al. (2016), Cell 166, 88-101). The fairly uniform abundance of C-end fragments among all fragment sizes suggests that DNASE1L3 can cleave all DNA, even intranucleosomal DNA efficiently.

DNASE1L3 has interesting properties: it is expressed in the endoplasmic reticulum to be secreted extracellularly as one of the major serum nucleases, and it translocates to the nucleus upon cleavage of its endoplasmic reticulum-targeting motif after apoptosis is induced (Errami, Y. et al. (2013), The Journal of Biological Chemistry 288, 3460-3468); Napirei, M. et al., (2005), The Biochemical Journal 389, 355-364)). In its role as an apoptotic intracellular endonuclease, it has been suggested that DNASE1L3 cooperates with DFFB in DNA fragmentation (Errami, Y. et al. (2013), The Journal of Biological Chemistry 288, 3460-3468); Koyama, R. et al., (2016), Genes to Cells 21, 1150-1163)). When comparing the fragment end profiles of fresh cfDNA with that of DNASE1L3-deficient mice, there is a noticeable attenuation of the periodicity in A-end fragments, and especially in the A< >C fragment. We suspect this attenuation is due to the coexisting intracellular activity of DNASE1L3 during the generation of freshly fragmented DNA from apoptosis in WT versus in DNASE1L3-deficient mice.

As a plasma nuclease, DNASE1L3 would help digest the DNA in circulation that had escaped phagocytosis after apoptosis. Hence, DNASE1L3 would likely exert its effect on fragmented cfDNA after intracellular fragmentation had occurred. In a theoretical two-step process, inhibiting the second step should reveal the usually transient outcome of the first step. So, in essence, the plasma of DNASE1L3-deficient mice would have this second step of DNASE1L3 action inhibited and expose the cfDNA profile of the first step, the intracellular DNA fragmentation from apoptosis. This is exactly what we found, with the cfDNA fragment profile remarkably similar to that found in freshly generated cfDNA. Thus, DNASE1L3 digestion within the plasma might a subsequent step that would result in the typical homeostatic cfDNA.

While we previously found that the size profile of cfDNA from DNASE1-deficient mice did not appear to be substantially different from that of WT mice, DNASE1 is known to prefer cleaving ‘naked’ DNA and can only cleave chromatin with proteolytic help in vivo (Cheng, T. H. T. et al., (2018), Clin Chem 64, 406-408; Napirei, M. et al., (2009), The FEBS Journal 276, 1059-1073)). Using heparin to replace the function of in vivo proteases to enhance DNASE1 activity, we have demonstrated that DNASE1 prefers to cut DNA into T-end fragments. The increase in T-end fragments with heparin incubation is predominantly subnucleosomally-sized (50-150 bp), suggesting that DNASE1 has a role in generating short <150 bp fragments. Knowing that DNASE1 prefers to cleave naked DNA into T-end fragments, we can infer from the typical cfDNA profile that the T-end fragment peaks in 50-150 bp and 250-300 bp range may be mostly naked. It may be possible since these sizes correspond to subnucleosomal fragments or linker fragments; however, more studies should be done to further investigate this hypothesis.

The use of heparin incubation and end analysis have also provided a unique insight into the origin of the 10 bp periodicity. Since every fragment type demonstrates a 10 bp periodicity, we show that no one specific nuclease is completely responsible for the 10 bp periodicity in short fragments. Instead, we demonstrate that for all fragment types, the 10 bp periodicity is abolished when heparin is used. In addition to enhancing DNASE1 activity, heparin disrupts the nucleosomal structure (Villeponteau, B. (1992), The Biochemical journal 288 (Pt 3), 953-958). While many have postulated that the 10 bp periodicity originates from the cutting of DNA within an intact nucleosomal structure, we believe that this work provides supportive evidence, showing that no 10 bp periodicity occurs in the presence of a disrupted nucleosome.

Recently, Watanabe et al. induced in vivo hepatocyte necrosis and apoptosis with acetaminophen overdose and anti-Fas antibody treatments in mice deficient in DNASE1L3 and DFFB (Watanabe, T. et al., (2019), Biochemical and biophysical research communications 516, 790-795). While Watanabe et al. claims to have shown that cfDNA is generated by DNASE1L3 and DFFB, their data only shows that serum cfDNA does not appear to increase after hepatocyte injury in DNASE1L3- and DFFB-double knockout mice. Even then, the degree of hepatocyte injury from their methods is hugely variable even in wildtype with surprisingly low correlation with cfDNA amount in their apoptotic anti-Fas antibody experiments. In addition to these inconsistencies that gives uncertainty to the degree of apoptosis induced in their knockout mice, they have none of the detail on fragment ends offered in this study.

In this study, we have demonstrated that the typical cfDNA fragment might be created in two major steps: 1) intracellular DNA fragmentation by DFFB, intracellular DNASE1L3, and other apoptotic nucleases, and 2) extracellular DNA fragmentation by serum DNASE1L3. Then, likely with in vivo proteolysis, DNASE1 can further degrade cfDNA into short T-end fragments. We believe that this first model has included a number of key nucleases involved in cfDNA generation, but the model can be further refined in the future. For example, other potential apoptotic nucleases include endonuclease G, AIF, topoisomerase II, and cyclophilins, with probably more to be discovered (Nagata, S. (2018), Annual Review of Immunology 36, 489-517; Samejima, K. and Earnshaw, W. C. (2005), Nature Reviews: Molecular Cell Biology 6, 677-688; Yang, W. (2011), Quarterly reviews of biophysics 44, 1-93). Further studies into these nucleases with double knockout models would further refine this model and may reveal a nuclease with G-end preference. In essence, in this work, we have definitively linked the action of distinct nucleases to the cfDNA fragment end profile, clarifying the fundamental biology and biography of cfDNA fragments.

With this link between nuclease biology and cfDNA physiology established, there are many practical implications to the field of cfDNA. Firstly, aberrations in nuclease biology with pathological consequences may be reflected in abnormal cfDNA profiles (Al-Mayouf et al. (2011), Nat Genet 43, 1186-1188; Jimenez-Alcazar, M. et al. (2017), Science 358, 1202-1206; Ozcakar, Z. B. et al., (2013), Arthritis Rheum 65, 2183-2189)). Secondly, plasma end motif analysis is a powerful approach for investigating cfDNA biology and may have diagnostic applications. And lastly, the pre-analytical variables such as anticoagulant type and time delay in blood separation are vital confounders to bear in mind when mining cfDNA for epigenetic and genetic information.

D. Effects of Differential Regulation of Nucleases on Jagged Ends in Cell Free DNA

For cell-free DNA molecules with jagged ends, the end motifs could be defined by the stretch of nucleotides in a single-stranded DNA molecule attached to a double-stranded DNA molecule. The length of such a single-stranded DNA molecule could be, for example but not limited to, 1 nt, 2 nt, 3 nt, 4 nt, 5 nt, 6 nt, 7 nt, 8 nt, 9 nt, and 10 nt or above. In one embodiment, nuclease-associated jagged ends would correspond to the nuclease recognition sites. In another embodiment, nuclease-associated jagged ends would correspond to jagged ends which are preferentially created by one or more nucleases. In another embodiment, nuclease-associated jagged ends would be defined by those jagged ends which are over-represented or under-represented in diseases.

In yet another embodiment, nuclease-associated jagged ends could be defined by those jagged ends which are over-represented or under-represented in nuclease knockout mice or other genetically modified animals. The quantity of jagged ends could be measured a number of technologies, including but not limited to approaches based on the filling of methylated or unmethylated cytosines during DNA end repair step (e.g., as described in U.S. Patent Publication No. 2020/0056245) or an approach based on the oligonucleotide probe-based hybridization (Harkins et al. Nucleic Acids Res. 2020; 48:e47). The quantity of jagged ends present in cell-free DNA molecules is referred to as the jaggedness index value. The jaggedness index value deduced by the filling of methylated cytosines during DNA end repair step [i.e. the percentage of methylated signals at CH sites (H: A, C, T) in read 2 of a paired-end sequencing reaction] is referred to as JI-M (i.e. Jaggedness index value-Methylated). The jaggedness index value deduced by the filling of unmethylated cytosines during DNA end repair step (i.e. the reduced percentage of unmethylated signals at CG sites in the read2) is referred to as JI-U (i.e. Jaggedness index value-Unmethylated).

### IV. End Signature Analysis Based on Differential Regulation of Nucleases

Although nuclease expression can be used to identify abnormal cells from normal cells, analyzing nuclease expression levels can involve invasive procedures. Further, techniques such as RNA sequencing can suffer from low accuracy. Given the above, it is challenging to safely and accurately detect nuclease expression for disease diagnosis purposes. To overcome these deficiencies, embodiments of the present disclosure determines that a particular nuclease (e.g., DNASE1) preferentially cuts DNA into DNA molecules having a particular sequence end signature, determine an amount of sequence reads that include the sequence end signature, and use the amount to predict a classification of the level of abnormality of a tissue corresponding to the biological sample.

A. Detecting Abnormal Cells in a Subject

In one embodiment, the nuclease-cleaved signatures (e.g., preferential cutting of certain nucleases) could be identified by analyzing plasma DNA end motifs (e.g., 4-nt sequences at the ends of plasma DNA) between subjects with and without cancers. In one embodiment, the motifs can be chosen based on the gene expression patterns of one or more nucleases and the preferred cleavage sequences of the one or more nucleases. In one example, as revealed in various nuclease-deleted mouse models (Han et al. Am J Hum Genet. 2020; 106:202-14), the DNASE1L3 enzyme is known to preferentially create 5′ C-end fragments when cutting DNA molecules, the DFFB enzyme is known to preferentially create 5′ A-end fragments when cutting DNA molecules, and the DNASE1 enzyme is known to preferentially create 5′ T-end fragments when cutting DNA molecules. In one embodiment, the end motifs ending with C could be defined as DNASE1L3-cutting signatures, the end motifs ending with A as DFFB-cutting signatures and the end motifs ending with T as DNASE1-cutting signatures.

Therefore, we hypothesized that the abundance of an end motif associated with a downregulated nuclease (e.g., DNASE1L3) normalized by that of an end motif associated with an upregulated nuclease (e.g., DFFB), or vice versa, would reflect the physiological or pathological state of the related tissues. In one embodiment, one could use other statistical and/or mathematical calculations to utilize one or more nuclease-cutting signatures, including but not limited to, relative/absolute deviations, relative/absolute percentage increases, relative/absolute percentage decreases, linear/non-linear combinations of multiple ratios or deviations, etc.

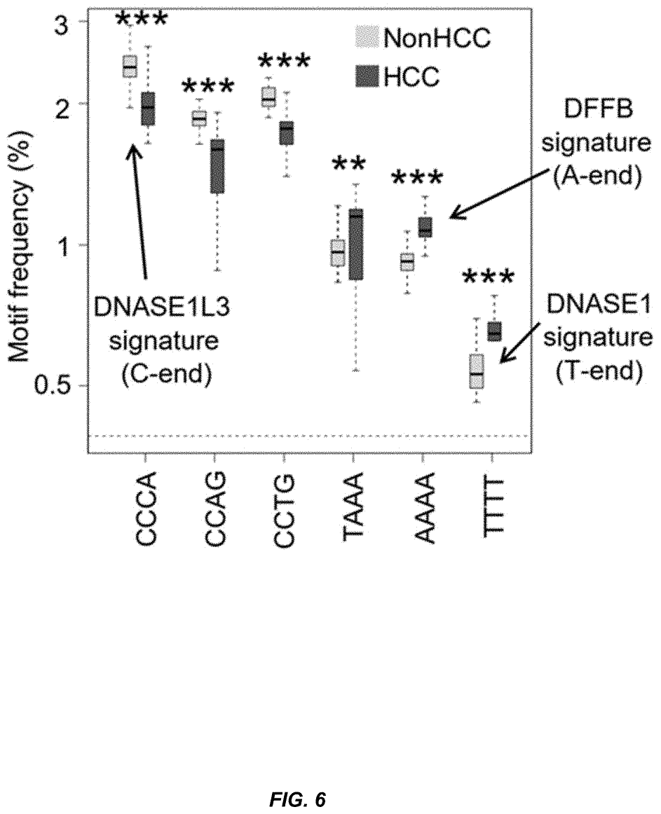

FIG. 6 shows an example distribution of cell-free DNA molecules with certain end signatures for determining the physiological or pathological state of a tissue, according to some embodiments. To this end, we focused on the end motifs with 5′ C-end (nuclease DNASE1L3 preferred) whose frequencies decreased in HCC subjects compared to healthy subjects, and end motifs with 5′ A-end (nuclease DFFB preferred) or T-end (nuclease DNASE1 preferred) whose frequencies were increased in HCC subjects compared to healthy subjects. In FIG. 6, the three asterisks *** represent a p value that is less than 0.001, and the two asterisks ** represent a p value that is less than 0.01. The gray dashed line indicates the frequency of 1/256. In one embodiment, compared with non-HCC subjects, CCCA end motif could be defined as a DNASE1L3-cutting signature, AAAA end motif could be defined as a DFFB-cutting signature, and TTTT could be defined as a DNASE1-cutting signature (FIG. 6). In one embodiment, one would focus on the end motifs with 3′ A-end, C-end, T-end or G-end or base compositions in other positions of a DNA fragment. For example, if the nuclease recognition sites with high binding affinity would be more conservative than cutting sites, the end signature signals focused on motifs occurring in binding sites would be more specific.

In some embodiments, plasma DNA end motif profiles are determined based on biological samples collected from patients with a disease and from patients those without the disease. In particular, the biological samples are analyzed to assess the nuclease expression profile of an organ affected in such disease. Additionally or alternatively, cell lines derived from certain tissues with or without certain disease can be analyzed to assess the nuclease expression levels and DNA end motifs upon induced cell apoptosis (e.g., through the use of pharmacological agents, antibodies, radiation, etc). In some instances, plasma DNA end motif profiles can be determined by altering gene expression in cell lines or animal subjects, e.g., siRNA to dampen expression of certain nuclease and then analyzing the resultant plasma DNA.

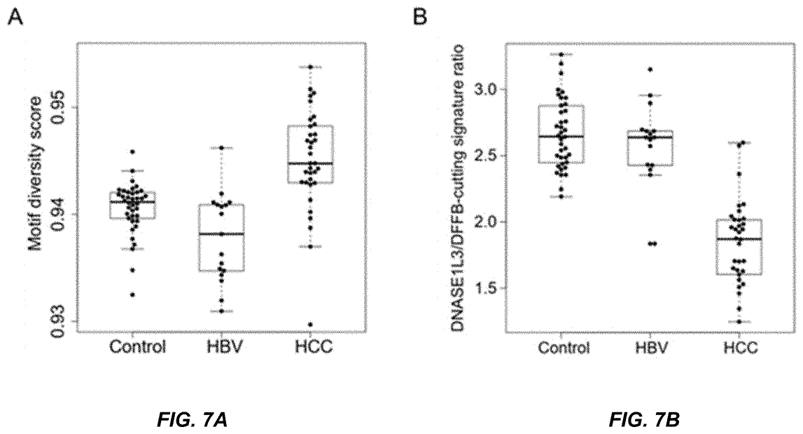

FIGS. 7A and 7B show boxplots that illustrate motif diversity scores and DNASE1L3/DFFB-cutting signature ratios across different tissue groups, according to some embodiments. In one embodiment, the ratio of DNASE1L3-cutting to DFFB-cutting signatures, referred to as a DNASE1L3/DFFB-cutting signature ratio, was used as one metric for diagnosis, for example, cancer detection. In addition, each of FIGS. 7A and 7B shows results for the following subject categories: (i) “Control”—healthy control subjects; (ii) “HBV”—chronic infection with hepatitis B virus; and (iii) “HCC”—subjects with hepatocellular carcinoma.

In one embodiment, the use of a DNASE1L3/DFFB-cutting signature ratio would misclassify only 8.8% of patients with HCC as normal subjects if one used the 5^{th} percentile of ratios in control subjects as a threshold. On the other hand, using the motif diversity score (MDS) would misclassify 29.4% of patients with HCC as normal subjects. The motif diversity score was defined as (Jiang et al. Cancer Discov. 2020; 10:664-673):

MDS=Σᵢ₌₁²⁵⁶−Pᵢ*log(Pᵢ)/log(256)

where Pi is the frequency of a particular motif. A higher MDS value indicates a higher diversity (i.e., a higher degree of randomness). The theoretical scale ranges from 0 to 1. Accordingly, the DNASE1L3/DFFB-cutting signature ratio provide for increased accuracy to classify subjects as having cancer, e.g., HCC.

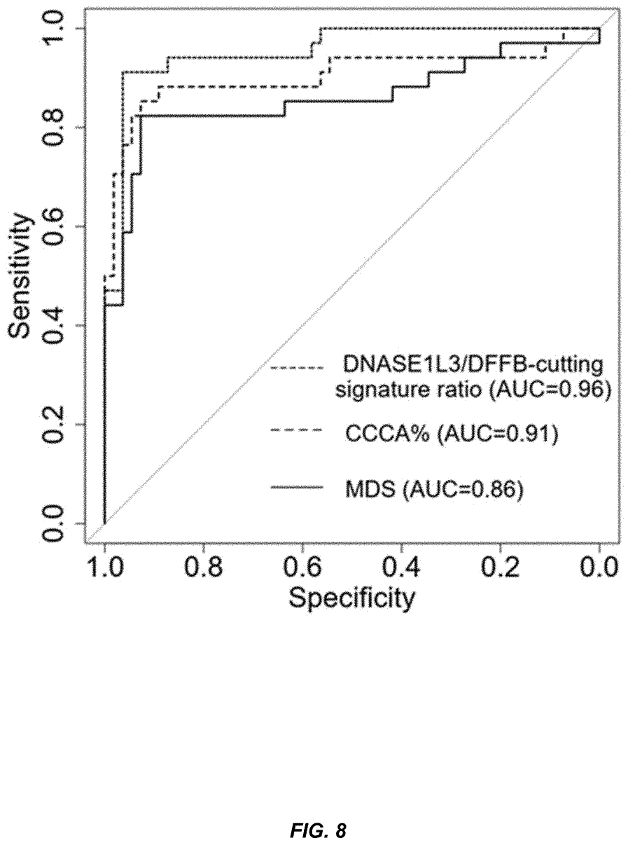

FIG. 8 shows receiver operating characteristic (ROC) curves for assessing different parameters for detection of end signatures, according to some embodiments. These results suggested that the performance using the DNASE1L3/DFFB-cutting signature ratio would be superior to that using the recently reported MDS metric (Jiang et al. Cancer Discov. 2020; 10:664-673). Such a conclusion was further supported by receiver operating characteristic curve (ROC) analysis (FIG. 8), in which the area-under-curve (AUC) of DNASE1L3/DFFB-cutting signature ratio-based analysis (AUC: 0.96) was greater to the MDS analysis (AUC: 0.86; P value <0.01, bootstrap test) and the CCCA % analysis (AUC: 0.91; P value=0.05, bootstrap test). These results suggested that the selection of motifs linking to the nucleases aberrant in tissues/organs of interest would improve the discriminative power in differentiating the patients with and without cancers, leading to better identification of the clinical status of the patients.

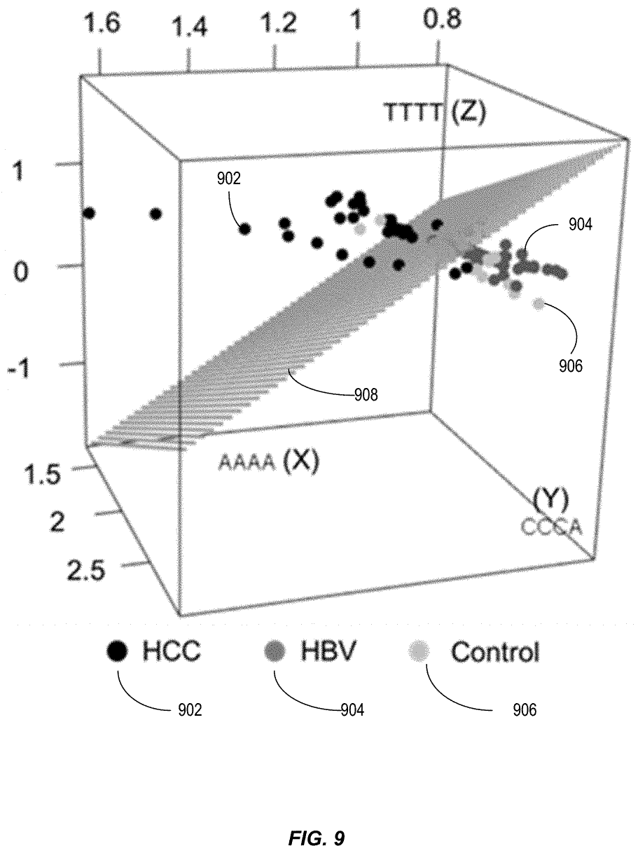

FIG. 9 shows a three-dimensional scatter plot of DNASE1L3-, DFFB- and DNASE1-cutting signatures in accordance with some embodiments. The x-axis indicates the DFFB-cutting signature (AAAA); the y-axis indicates the DNASE1L3-cutting signature (CCCA); and the z-axis indicates the DNASE1-cutting signature (TTTT). Further, dots 902 (e.g., “HCC”) represent end-cutting signatures of subjects with HCC, dots 904 (e.g., “HBV”) represents end-cutting signatures of subjects with chronic HBV infection, and dots 906 (e.g., “Control”) represents end-cutting signatures of healthy subjects. The shaded region 908 indicates a classifying hyperplane which was used for differentiating subjects with and without cancer.

As shown in FIG. 9, more than two nuclease-cutting signatures are used to carry out the assessment, including but not limited to DNASE1L3, DFFB, and DNASE1 nucleases. As shown in FIG. 9, HCC subjects deviated from non-HCC subjects including healthy controls and patients with chronic HBV infection. If we set a classifying hyperplane (−8.6*x+2.6*y−3.2*z+4.8=0) in a 3-dimensional plot, we could achieve 91.1% sensitivity and 96.4% specificity for discriminating between HCC and subjects with HBV or healthy controls. In one embodiment, the use of nuclease-cutting signatures in plasma DNA would serve as prognostic markers for monitoring patient responses during therapies, including chemotherapy, radiotherapy, immunotherapy, and targeted therapy.

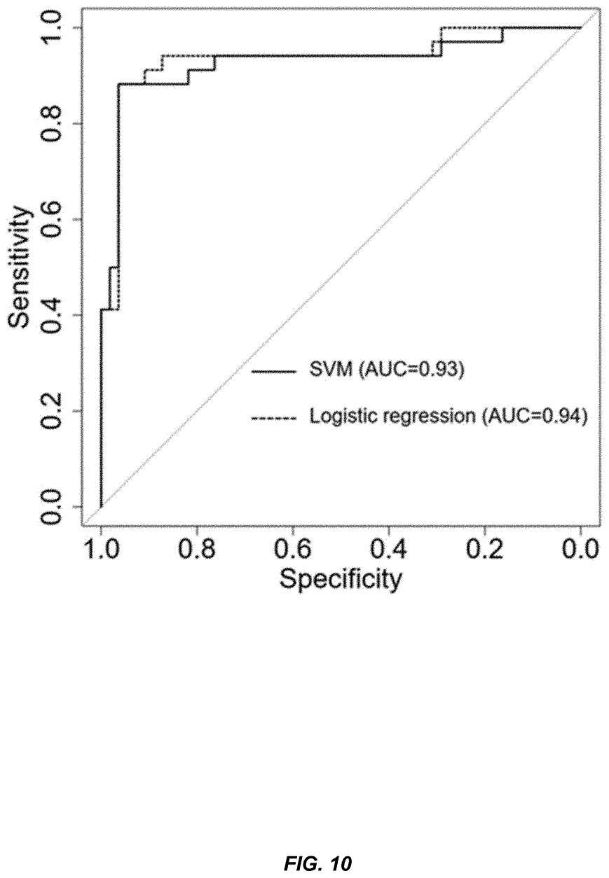

FIG. 10 shows an ROC graph depicting performance levels of using logistic regression to determine DNASE1L3-, DFFB-, and DNASE1-cutting signatures, according to some embodiments. In one embodiment, we could employ different statistical approaches to selectively make use of a number of nuclease-cutting signatures, for example but not limited to, including logistic regression, support vector machines (SVM), decision tree, naïve Bayes classification, clustering algorithm, principal component analysis, singular value decomposition (SVD), t-distributed stochastic neighbor embedding (tSNE), artificial neural network, and ensemble methods which construct a set of classifiers and then classify new data points by taking a weighted vote of their prediction. As shown in FIG. 10, by using logistic regression analysis and SVM model by taking advantage of three cutting end signatures of three nucleases (e.g., DNASE1L3, DFFB, and DNASE1), subjects with HCC could be differentiated from non-HCC subjects with an AUC of 0.94 and 0.93, respectively. We achieved 94% sensitivity and 93% specificity using a regression score of 0.85.

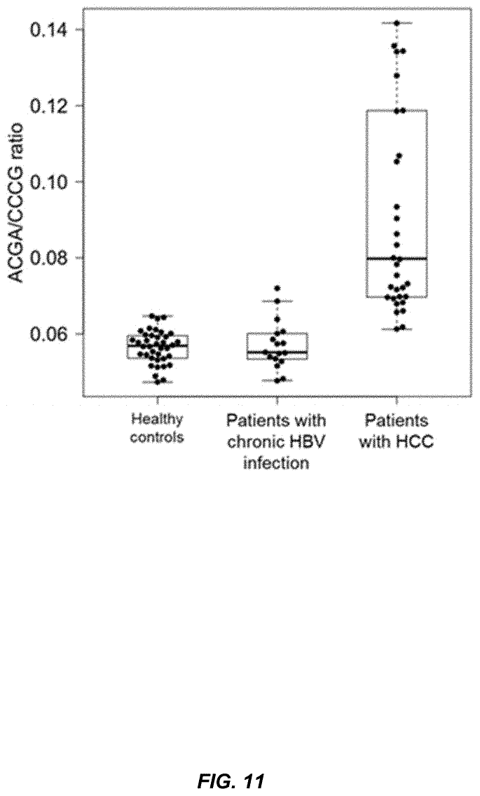

FIG. 11 shows a boxplot depicting the ratio of two plasma end motifs (ACGA/CCCG) according to some embodiments. In one embodiment, we could define nuclease-cutting signatures by enumerating all combinations of plasma DNA end signatures to determine the optimal combination for differentiating the patients with and without diseases that were associated with the aberrant profile of nuclease activities, including organ transplantations, pregnancy, cancers, immune-related disorders, and other diseases. As an example, one could enumerate all combinations concerning frequency ratios between any two end motifs. There are 256 motifs, leading to 32,640 combinations. Among 32,640 frequency ratios between any two end motifs, the frequency ratio of the ACGA to CCCG end motifs would increase in patients with HCC (FIG. 11), giving the most discriminative power in differentiating patients with (n=34) and without HCC (n=55), with an AUC of 0.99.

On the other hand, for detecting patients with other cancers including colorectal cancer, lung cancer, nasopharyngeal carcinoma, and head and neck squamous cell carcinoma, the frequency ratio of the AGTA to TCAA end motifs gave the most discriminative power, with an AUC of 0.98. In one embodiment, the frequency ratio of the AGTA to TCAA end motifs gave the highest AUC of 0.99 when differentiating patients with and without colorectal cancers. The frequency ratio of the CATC to GAGA end motifs gave the highest AUC of 1 when differentiating patients with and without lung cancers. The frequency ratio of the CACT to GAAC end motifs gave the highest AUC of 1 when differentiating patients with and without head and neck squamous cell carcinoma.

1. End-Signature Ratio Analysis Between Wildtype Mice and DNASE1L3-Deleted Mice

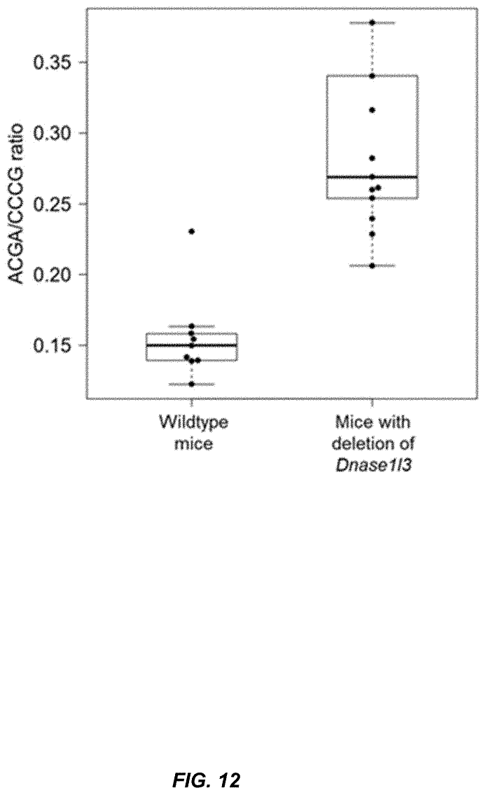

FIG. 12 shows a boxplot depicting the ratio of two plasma end motifs (ACGA/CCCG) between wildtype mice and DNASE1L3-deleted mice, according to some embodiments. In one embodiment, we could define or confirm nuclease-cutting signatures by analyzing 4-nt end motifs between the mice with and without deletion of one or more nuclease genes such as, but not limited to, DNASE1L3, DFFB, and DNASE1. For example, the increase of the ratio of ACGA to CCCG end motifs was also confirmed in mice with the deletion of DNASE1L3 (FIG. 12). These results suggested that the alteration of a certain end motif ratio that was potentially caused by the downregulation of DNASE1L3 in patients with HCC could be orthogonally mirrored in mice with the deletion of DNASE1L3. In one embodiment, such orthogonal confirmation of the changing patterns of end motif ratios would allow determining the informative end motif ratios for human clinical assessments.

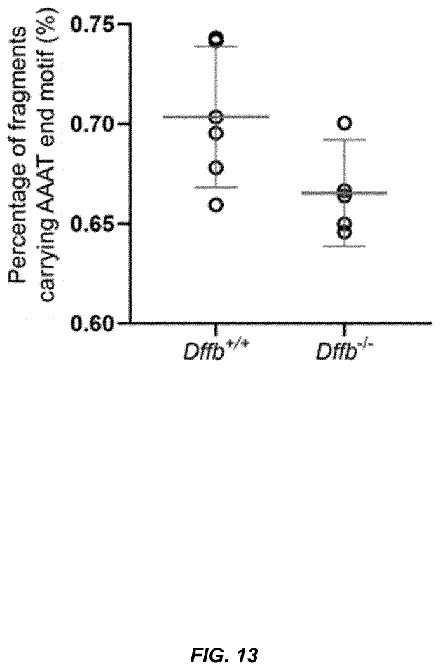

FIG. 13 shows percentage of plasma DNA fragments carrying AAAT end motif between wildtype (DFFB^{+/+}) and DFFB deletion mice (DFFB^{-/-}), according to some embodiments. In one embodiment, as shown in FIG. 13, the frequency of molecules carrying AAAT end motif in plasma DNA of mice with the deletion of DFFB (DFFB^{-/-}) (median: 0.70%; range: 0.66-0.74%) was found to be lower than that of wildtype mice (DFFB^{+/+}) (median: 0.66%; range: 0.64-0.7%).

2. End-Signature Ratio Analysis Between Normal and Abnormal Cells of Human Subjects

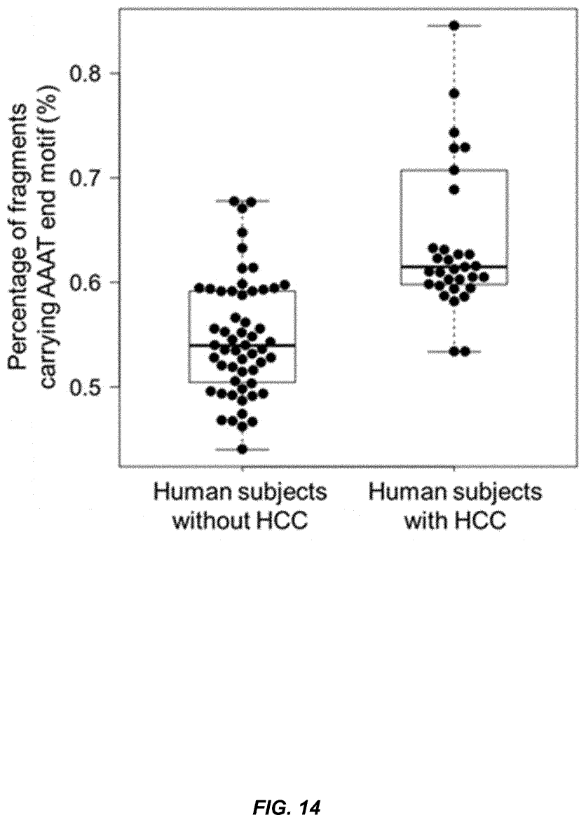

FIG. 14 shows a percentage of plasma DNA fragments carrying AAAT end motif between human subjects with and without HCC, according to some embodiments. Such AAAT end motif was found to be elevated in human patients with HCC, compared with subjects without HCC (FIG. 14). Considering the relative elevation of DFFB expression in HCC tissues (FIG. 4B), end motif AAAT can be deemed as a DFFB-cutting signature in one embodiment.

In some embodiments, a particular end motif (e.g., AAAT) is selected from a plurality of known end motifs, based on a determination that an increased or decreased amount of the particular end motif substantially corresponds to a respective increased or decreased amount of a corresponding nuclease (e.g., DFFB). Additionally or alternatively, different statistical approaches can be employed to selectively identify end motifs that are likely to represent a cutting signature for a corresponding nuclease. The different statistical approaches can include, but are not limited to, including logistic regression, support vector machines (SVM), decision tree, naïve Bayes classification, clustering algorithm, principal component analysis, singular value decomposition (SVD), t-distributed stochastic neighbor embedding (tSNE), artificial neural network, ensemble methods which construct a set of classifiers and then classify new data points by taking a weighted vote of their prediction.

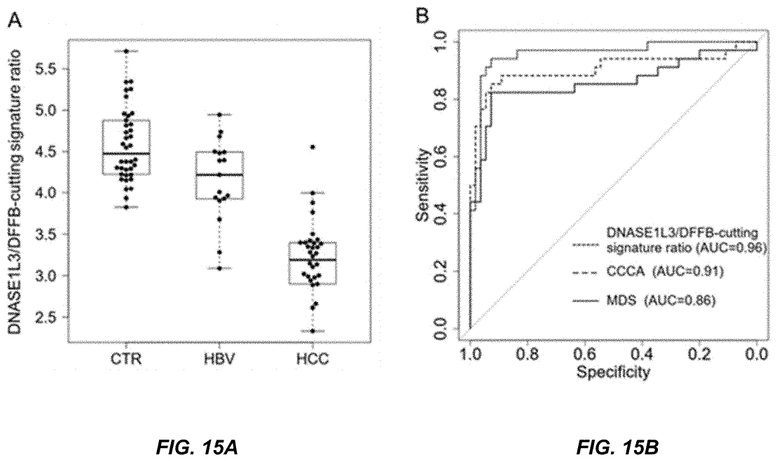

FIG. 15A shows a boxplot of DNASE1L3/DFFB-cutting signature ratio values across human healthy control subjects (CTR), subjects with chronic hepatitis B infection (HBV) and subjects with HCC (HCC), and FIG. 15B shows ROC curves between patients with and without HCC using DNASE1L3/DFFB-cutting signature ratio (densely dashed line), percentage of fragments with end motif CCCA (CCCA, loosely dashed line) and motif diversity score (MDS, solid line), in accordance with some embodiments. In some instances, one could define the ratio between end motifs CCCA and AAAT in plasma DNA as DNASE1L3/DFFB-cutting signature ratio.

FIG. 15A shows there were lower DNASE1L3/DFFB-cutting signature ratios present in plasma of patients with HCC, compared with healthy control and hepatitis B virus carriers. FIG. 15B shows that the DNASE1L3/DFFB-cutting signature ratio metric (area-under-the-curve (AUC): 0.96) was superior to CCCA end motifs (AUC: 0.91) and MDS (AUC: 0.86). These results suggested that one could use information regarding an end motif which would be preferentially cut by a nuclease (e.g., CCCA motif preferentially cut by DNASE1L3) and an end motif altered in mice whose nuclease (e.g., DFFB) was genotypically modified to devise a new method for more effectively differentiating patients with and without HCC, other cancers and indeed other diseases. IOther embodiments can be applied to other nucleases, including, but not limited to, TREX1, AEN, EXO1, DNASE2, DNASE1, ENDOG, APEX1, FEN1, DNASE1L1, DNASE1L2 and EXOG.

3. End-Signature Ratio Analysis Between Pregnant Subjects with or without Preeclampsia

It is shown that certain nucleases can be differentially regulated in subjects with preeclampsia relative to subjects without preeclampsia. For example, by analyzing the microarray-based gene expression profiling datasets in previously published studies (Nishizawa et al. Reprod Biol Endocrinol. 2011; 9:107; Gormley et al. Am J Obstet Gynecol. 2017; 217: 200.e1-200.e17), the DNASE1L3 expression level was found to be downregulated by 6% in pregnant subjects with preeclampsia, in comparison with control pregnant subjects with normal blood pressure. Conversely, the DNASE1 expression level was found to be upregulated by 5.7% in pregnant subjects with preeclampsia compared with the non-infected preterm birth. As such, one or more end-cutting signatures of a particular nuclease can be used to determine a parameter that is predictive of whether a pregnant subject has preeclampsia.

The ratio between DNASE1-cutting end signatures (e.g., fragments terminated with a thymine nucleotide) and DNASE1L3-cutting end signatures (e.g., fragments terminated with a cytosine nucleotide) can be used to differentiate between pregnant women with and without preeclampsia.

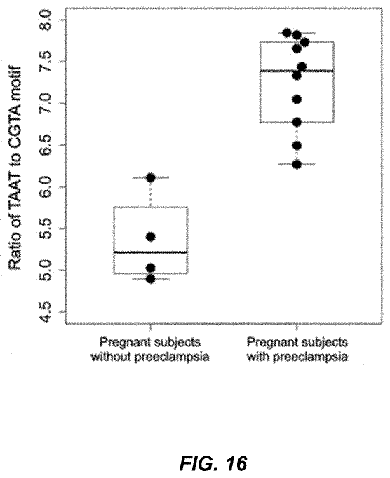

FIG. 16 shows a boxplot of DNASE1/DNASE1L3-cutting signature ratio values across control subjects (e.g., pregnant subjects without preeclampsia) and pregnant subjects with preeclampsia. In FIG. 16, DNASE1-cutting end signature corresponds to sequence TAAT, and DNASE1L3-cutting end signature corresponds to CGTA. Next generation sequencing (short-read paired-end sequencing, Illumina) was used to sequence pregnant subjects with (n=4) and without preeclampsia (n=10), with a median of 42 million mapped reads (range: 21-50 million).

Continuing with the example shown in FIG. 16, the median ratio of TAAT to CGTA end motif frequency of pregnant women with preeclampsia (median: 7.39; range: 6.27-7.84) is higher than the median ratio of control subjects (median: 5.21; range: 4.90-6.11) (P value=0.001; Mann-Whitney U test). Thus, DNASE1/DNASE1L3-cutting signature ratio values can be advantageous in distinguishing pregnant women with preeclampsia from those without preeclampsia.

4. Methods for Determining Level of Abnormality in Tissue Type

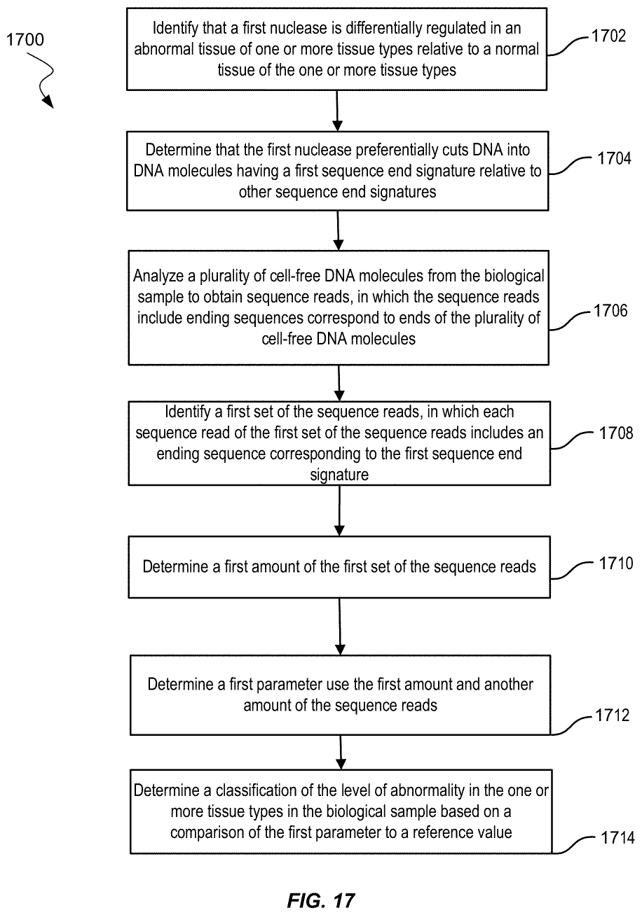

FIG. 17 is a flowchart illustrating a method for classifying a level of abnormality in a biological sample based on sequence end signatures, according to some embodiments. In some instances, the biological sample includes cell-free DNA molecules. The abnormality may be a pathology including cancer (e.g., hepatocellular carcinoma, lung cancer, breast cancer, gastric cancer, glioblastoma multiforme, pancreatic cancer, colorectal cancer, nasopharyngeal carcinoma, head and neck squamous cell carcinoma, etc.) and an auto-immune disorder (e.g., systemic lupus erythematosus). In some instances, the abnormality in the biological sample is an abnormality of placental tissue (e.g., placental tissue detected in maternal plasma), including preeclampsia, preterm birth, fetal chromosomal aneuploidies, or fetal genetic disorders.

At step 1702, a first nuclease being differentially regulated in abnormal cells of one or more tissue types relative to a normal tissue of the one or more tissue types is identified. For example, DNASE1L3 expression is relatively downregulated in HCC cells compared with liver tissues in healthy subjects. In some instances, a second nuclease being differentially regulated in an abnormal tissue cells of one or more tissue types relative to a normal tissue of the one or more tissue types is also identified. For example, DFFB and DNASE1 expression are relatively upregulated in in HCC cells compared with liver tissues in healthy subjects.

At step 1704, the first nuclease is determined to preferentially cut DNA into DNA molecules having a first sequence end signature relative to other sequence end signatures. For example, the nuclease-cleaved signatures could be identified by analyzing plasma end motifs (e.g., 4-nt sequences at the ends of plasma DNA) between subjects with and without cancers. In some instances, the cutting preference of the first nuclease is determined by analyzing a biological sample of another organism (e.g., mice).

At step 1706, a plurality of cell-free DNA molecules from the biological sample are analyzed to obtain sequence reads. In some embodiments, paired-end sequencing is used to obtain two sequence reads from the two ends of a DNA fragment, e.g., 30-120 bases per sequence read. As described herein, sequence read may be obtained in a variety of ways, e.g., using sequencing techniques (e.g., using a sequencing-by-synthesis approach (e.g., Illumina), or single molecule sequencing (e.g., by the single molecule, real-time system from Pacific Biosciences, or by nanopore sequencing (e.g., by Oxford Nanopore Technologies), or using probes, e.g., in hybridization arrays or capture probes. In some embodiments, the sequencing process may be preceded by amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed. As examples, the analysis can use probe-based or sequence-based techniques, as are described herein.

At step 1708, a first set of the sequence reads is identified. In some embodiments, each sequence read of the first set of the sequence reads includes an ending sequence corresponding to the first sequence end signature. In some embodiments, the first set of sequence reads include ending sequences corresponding to ends of the plurality of cell-free DNA molecules. The ending sequences having the first sequence end signature may be determined using a reference genome, e.g., to identify bases just before a start position or just after an end position. Such bases will still correspond to ends of cell-free DNA fragments, e.g., as they are identified based on the ending sequences of the fragments.

At step 1710, a first amount of the first set of the sequence reads is determined. In some embodiments, the first amount of the first set of the sequence reads may be counted (e.g., stored in an array in memory).

At step 1712, a first parameter is determined by using the first amount and potentially another amount of the sequence reads. In some examples, both of such amounts can be separate parameters. The other amount can take various forms, e.g., corresponding to a total number of sequence reads and/or DNA molecules analyzed. As another example, the other amount can correspond to an amount of a second set of sequence reads that each include an ending sequence corresponding to one or more other sequence end signatures (end motifs). Thus, the first parameter can be a ratio of amounts between two sets of sequence reads having their respective end motifs. In such examples, the other amount can normalize the first amount so as to provide consistent measurements, regardless of the sample size or number of DNA molecules analyzed. Such normalization can result in a normalized parameter, which provides a relative amount between the first amount the other amount (e.g., a ratio of the amounts or a ratio of functions of the amounts).

In some instances, the first parameter (e.g., DNAS1L3/DFFB) is generated by using the first amount of sequence reads that include ending sequences corresponding to an end signature of the first nuclease (e.g., DNAS1L3) and a second amount of sequence reads that include ending sequences corresponding to an end signature of the second nuclease (e.g., DFFB), in which the second nuclease is differentially regulated in an abnormal tissue cells of one or more tissue types relative to a normal tissue of the one or more tissue types. Accordingly, in various examples, the first parameter can include a motif diversity score, relative frequencies of end motifs, or DNASE1L3/DFFB-cutting signature ratio.

Differences in relative frequencies of end motifs can be detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

In some instances, the same amount of sequence reads is used for normalizing each parameter that represents expression levels of a corresponding nuclease. Additionally or alternatively, different amounts of sequence reads can be used to normalize each parameter for a corresponding nuclease.

At step 1714, a classification of the level of abnormality in the one or more tissue types in the biological sample is determined, in which the determination of the classification of the level of abnormality is based on a comparison of the first parameter to a reference value. For example, an increased value corresponding to a ratio of the ACGA to CCCG end motifs would indicate a classification of Hepatocellular carcinoma (HCC). In some embodiments, the classification of the level of abnormality includes one of a plurality of stages of pathology (e.g., HCC).

In some embodiments, parameters generated based on respective nucleases can thus be used to classify the level of abnormality. These respective parameters can be combined to form a new combined parameter, e.g., as a ratio, a ratio of respective functions of the respective parameters, and as two inputs to more complex functions, such as a machine learning model. Example combined parameters can include DNASE1L3/DFFB, DNASE1/DFFB, or other ratios of DNASE1L3:DNASE1:DFFB. Further, the parameters of more than two nucleases can be used, e.g., relative parameters of 3 or more nucleases can be used.

In some embodiments, the classification of the level of abnormality can be determined based on analyzing a set of parameters, in which each parameter corresponds to an amount of sequence reads that each include an ending sequence corresponding to a particular sequence end signature in combination with another amount (e.g., for normalization). For instance, a parameter can include a particular combination of frequency ratios between two sets of sequence reads with their respective end signatures. For example, a first parameter of the set of parameters may correspond to a ratio of end signatures (e.g., CCCA/AAAT) between a first amount of sequence reads each including an ending sequence corresponding to an end signature of a first nuclease and another amount of sequence reads, and a second parameter of the set of parameters may correspond to a ratio of end signatures (e.g., ACGA/CCCG) between a second amount of sequence reads each including an ending sequence corresponding to an end signature of a second nuclease and a third amount of sequence reads. In some instance, the third amount of sequence reads is the other amount sequence reads used to determine the first parameter.

In some examples for implementing steps 1712 and 1714, the first amount and the second amount can be input to a machine learning model (e.g., as described herein). The machine learning model can generate the parameter internally (e.g., as an intermediate value) and provide an output classification based on the two amounts. A training set can be developed from samples having one or more known levels of abnormality. The training of the machine learning model can provide the reference value as well as the formulation for how the first parameter is determined.

B. Fractional Concentration of Clinically-Relevant DNA

It was reported that the end motif profiles were different between fetal and maternal DNA molecules, as MDS values were lower in fetal DNA molecules than that in maternal DNA molecules (Jiang et al. Cancer Discov. 2020; 10:664-673). To test if the nuclease-cutting signature analysis in pregnant women would improve the signals for distinguishing the fetal DNA molecules from the maternal DNA molecules, we calculated the frequency ratio of the CCCA to AAAA end motifs (i.e. DNASE1L3/DFFB-cutting signature ratio).

1. Differentiation Between Maternal and Fetal DNA Using End-Signature Ratio Analysis

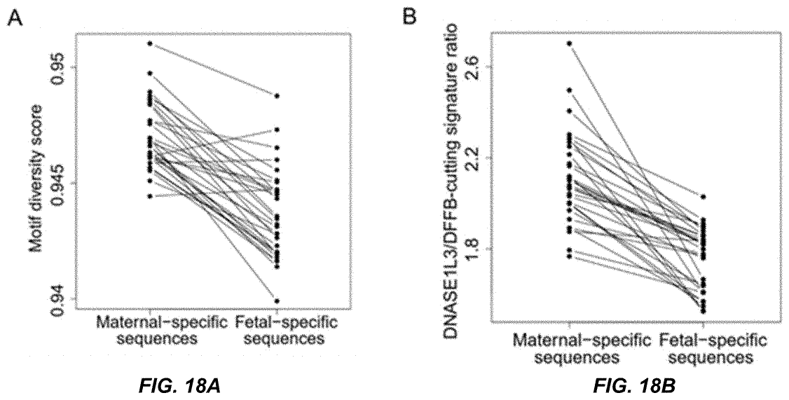

FIGS. 18A and 18B show examples of differentiating maternal and fetal DNA molecules using motif diversity score and DNASE1L3/DFFB-cutting signature ratio, according to some embodiments. As shown in FIGS. 18A and 18B, fetal-specific sequences generally corresponds to a lower motif diversity score and DNASE1L3/DFFB-cutting signature ratio than those of the maternal-specific sequences. However, the relative difference in measured values between maternal- and fetal-specific sequences is greater in DNASE1L3/DFFB-cutting signature ratio, compared to the motif diversity score. Thus, DNASE1L3/DFFB-cutting signature ratio can demonstrate a greater discriminative power in differentiating maternal- and fetal-specific sequences.

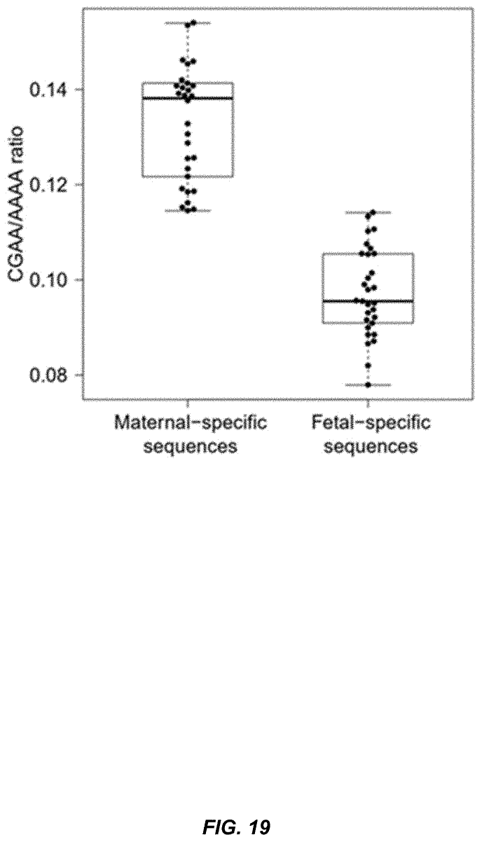

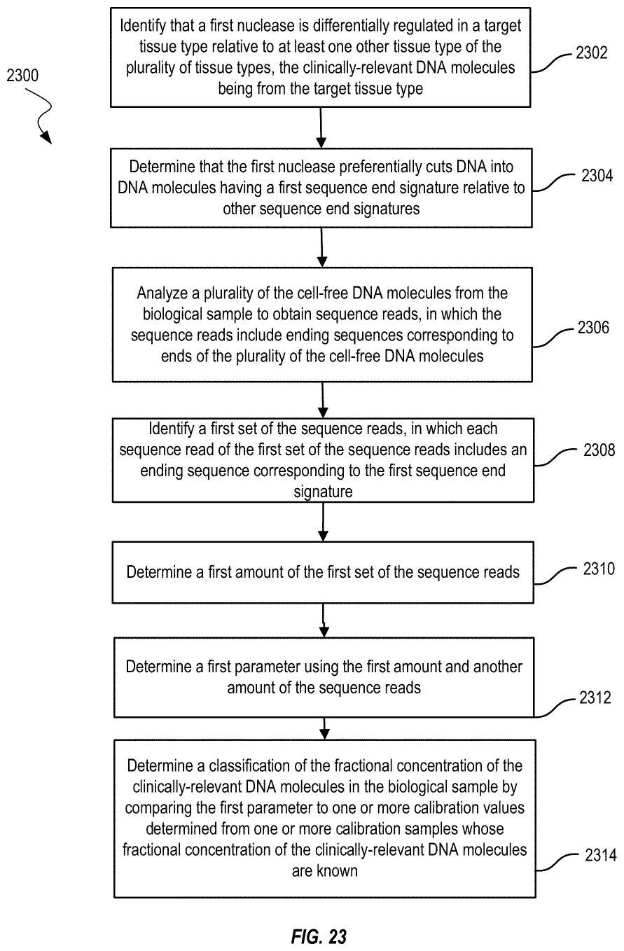

FIG. 19 shows a boxplot of the ratio of two plasma end motifs (CGAA/AAAA) for differentiating fetal and maternal DNA molecules, in accordance with some embodiments. In one embodiment, we could define nuclease-cutting signatures by using a permutation analysis to determine the combination of cutting signatures exhibiting the most discriminative power in differentiating fetal DNA molecules from maternal background DNA molecules. As an example, one could enumerate all combinations of frequency ratios between any two end motifs. There are 256 motifs, leading to 32,640. Among 32,640 frequency ratios between any two end motifs, the frequency ratio of the CGAA to AAAA end motif was decreased in fetal DNA molecules, showing an AUC of 1 between fetal and maternal DNA molecules (FIG. 23). These results suggested that the selective analysis of two particular end motifs (e.g., end motif ratio) would improve the discriminative power in determining the tissue of origin of plasma DNA molecules.

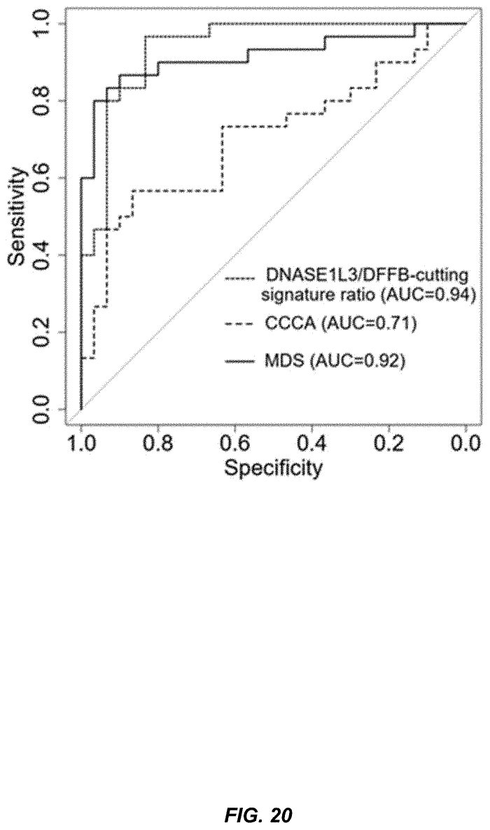

FIG. 20 shows ROC curves for MDS, CCCA % and DNASE1L3/DFFB-cutting signature ratio in differentiating maternal and fetal DNA molecules, according to some embodiments. The values corresponding to MDS, CCCA %, and cutting signature ratio were determined by a set of reads. Initially, maternal fragments and fetal fragments for each plasma sample of pregnant woman were identified based on SNP sites. The SNPs where the mother is homozygous (AA) and the fetus is heterozygous (AB) allow identifying the fetal-specific DNA molecules. The SNPs where the mother is heterozygous (AB) and the fetus is homozygous (AA) allow identifying the maternal-specific DNA molecules (i.e. maternal DNA).

For each plasma DNA sample, two cutting ratio values were obtained: one for the maternal DNA (X) and the other for fetal DNA (Y). For example, if we analyzed 30 pregnant subjects, there would be 30×values and 30 Y values. If the fetal and maternal DNA have different cutting preference, X and Y should be different. Using ROC between X and Y values, we aimed to illustrate which feature (e.g. MDS, CCCA % and DNASE1L3/DFFB-cutting ratio) would lead to the biggest difference between the sets of maternal and fetal DNA molecules. The higher AUC in the ROC indicated that the corresponding feature would be more powerful to reflect the maternal/fetal DNA contributions or maternal/fetal DNA related cutting alterations in plasma DNA pool. As such, the ROC curves in FIG. 20 are used for illustrating the feature importance of MDS, CCCA %, and the end-cutting signature ratio in being able to discriminate between maternal and fetal DNA, thereby being able to provide a fetal fractional concentration in methods described herein.

Compared with an AUC of 0.92 based on motif diversity score values between the fetal and maternal DNA molecules (FIG. 18A and FIG. 20), the frequency ratios of the CCCA to AAAA end motifs (i.e. DNASE1L3/DFFB-cutting signature ratio) gave rise to a higher AUC (0.94) (FIG. 18B and FIG. 20). The measure of CCCA % (i.e., DNASE1L3-cutting signature) gave the least discriminative power (AUC: 0.71). Accordingly, MDS and the DNASE1L3/DFFB-cutting signature ratio can provide good accuracy for being able to differentiate between maternal and fetal DNA molecules.

2. Tissue Differentiation

It was also reported that the end motif profiles were different between liver-derived DNA molecules and DNA molecules mainly of hematopoietic origin, as MDS values were lower in liver-derived DNA molecules than that in hematopoietically-derived DNA molecules (Jiang et al. Cancer Discov. 2020; 10:664-673). To test if the nuclease-cutting signature analysis in patients with liver transplantation would improve the signals for distinguishing the liver-derived DNA molecules from the DNA molecules mainly of hematopoietic origin, we also calculated the frequency ratio of the CCCA to AAAA end motifs.

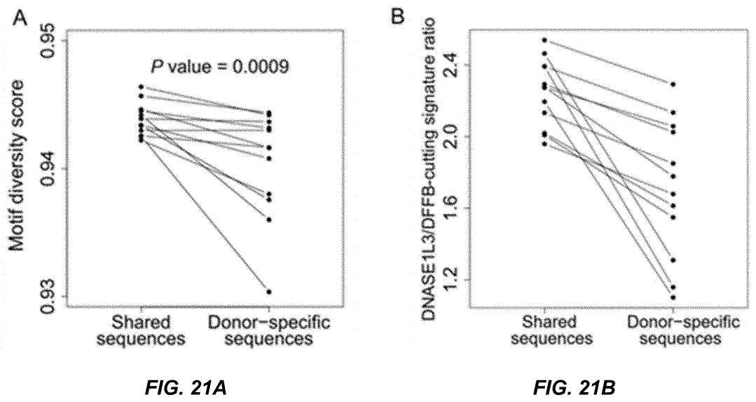

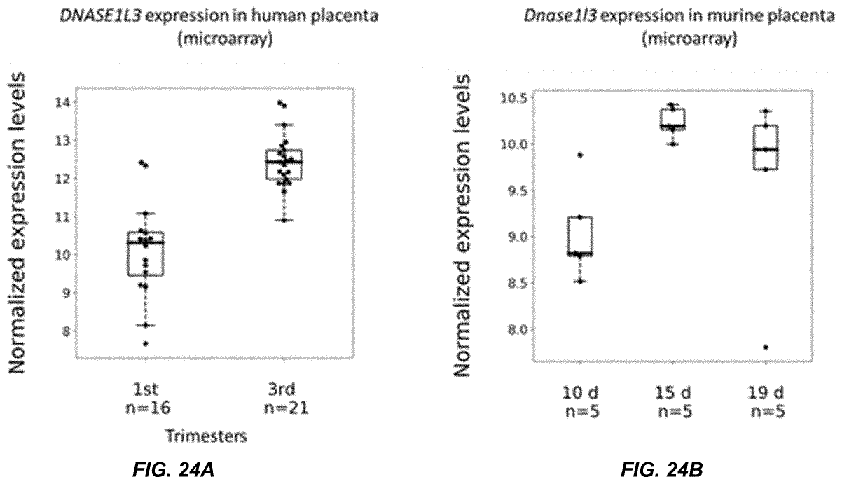

FIGS. 21A and 21B show examples of differentiating liver-derived DNA molecules and DNA molecules of hematopoietic origin using motif diversity score and DNASE1L3/DFFB-cutting signature ratio, according to some embodiments. As shown in FIGS. 24A and 24B, liver-derived sequences (e.g., donor-specific sequences) generally corresponds to a lower motif diversity score and DNASE1L3/DFFB-cutting signature ratio than those of the sequences of hematopoietic origin (e.g., shared sequences). However, the relative difference in measured values between the two sequences is greater in DNASE1L3/DFFB-cutting signature ratio, compared to the motif diversity score. Thus, DNASE1L3/DFFB-cutting signature ratio can demonstrate a greater discriminative power in differentiating maternal- and fetal-specific sequences.

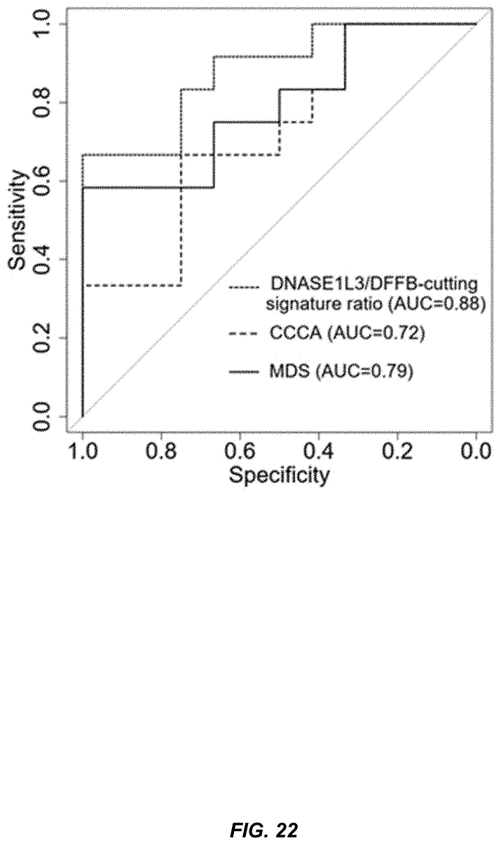

FIG. 22 shows ROC curves for MDS, CCCA % and DNASE1L3/DFFB-cutting signature ratio in differentiating liver-derived DNA molecules and DNA molecules of hematopoietic origin, according to some embodiments. Here, we used the plasma DNA samples of patients with liver transplantation. Initially, liver-derived DNA molecules and DNA molecules of hematopoietic origin were identified based on SNPs where the donor and recipient subjects have different genotypes (e.g. the donor's genotype AA and the recipient's genotype AB; or the donor AB and the recipient AA) for each plasma sample of liver transplantation patient.

Similar to the techniques used in FIG. 20, the ROC curves were used to illustrate which feature (e.g. MDS, CCCA % and DNASE1L3/DFFB-cutting ratio) would lead to the biggest difference between liver-derived DNA molecules and DNA molecules of hematopoietic origin (i.e. recipient-specific DNA). The higher AUC in the ROC indicated that the corresponding feature would be more powerful to reflect the liver-derived DNA contributions or liver-derived DNA related cutting alterations in plasma DNA pool.

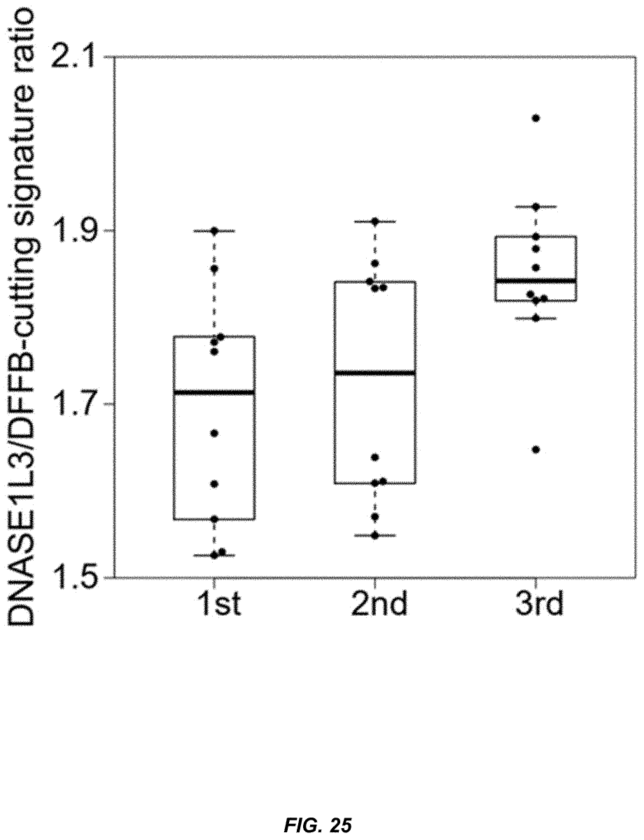

Compared with an AUC of 0.76 for MDS analysis between the liver-derived and hematopoietic DNA molecules (FIG. 24A and FIG. 25), the frequency ratios of the CCCA to AAAA end motif gave rise to a higher AUC (0.88) (FIG. 24B and FIG. 25). CCCA % gave the least discriminative power (AUC: 0.72). Accordingly, MDS and the DNASE1L3/DFFB-cutting signature ratio can provide good accuracy for being able to differentiate between liver-derived DNA molecules and DNA molecules of hematopoietic origin.

In one embodiment, nuclease-cutting signatures are defined by using a permutation analysis to determine the combination of cutting signatures exhibiting the most discriminating power in differentiating liver-derived DNA molecules from DNA molecules mainly of hematopoietic origin. As an example, one could enumerate all combinations of frequency ratios between any two end motifs. There are 256 motifs, leading to a total of 32,640 combinations. Among 32,640 frequency ratios between any two end motifs, the frequency ratio of the CTGA to GGAG end motif gave an AUC of 1. These results suggested that the selective analysis of two particular motifs would improve the discriminative power in differentiating the tissue of origin of plasma DNA molecules.

3. Methods for Determining Fractional Concentration of Clinically-Relevant DNA

FIG. 23 is a flowchart illustrating a method 2300 for estimating a fractional concentration of clinically-relevant DNA molecules in a biological sample, based on sequence end signatures in accordance with some embodiments. The biological sample includes a mixture of cell-free DNA molecules from a plurality of tissue types. In some embodiments, the clinically-relevant DNA includes fetal DNA, tumor DNA, or DNA of a transplanted organ. The target tissue type can include a liver tissue, hematopoetic cells, a fetal tissue, an organ that has a cancer, and a placental tissue. Similar steps in method 2300 can be performed in a similar manner as method 1700 of FIG. 17. Additionally, other methods with similar steps can be performed in a similar manner. Thus, additional description may not be repeated for each method.

At step 2302, a first nuclease is differentially regulated in a target tissue type relative to at least one other tissue type of the plurality of tissue types is identified. In some embodiments, the clinically-relevant DNA molecules are from the target tissue type. In some instances, a second nuclease being differentially regulated in the target tissue type of one or more tissue types relative to at least one other tissue type of the plurality of tissue types is also identified. Step 2302 may be performed in a similar manner as step 1702 of FIG. 17.

At step 2304, the first nuclease is determined to preferentially cut DNA into DNA molecules having a first sequence end signature relative to other sequence end signatures. In some instances, the cutting preference of the first nuclease is determined by analyzing a biological sample of another organism (e.g., mice).

At step 2306, a plurality of the cell-free DNA molecules from the biological sample are analyzed to obtain sequence reads. In some embodiments, the sequence reads include ending sequences corresponding to ends of the plurality of the cell-free DNA molecules. In some embodiments, paired-end sequencing is used to obtain sequence reads, which two sequence reads are obtained from the two ends of a DNA fragment, e.g., 30-120 bases per sequence read. As described herein, sequence read may be obtained in a variety of ways, e.g., using sequencing techniques (e.g., using a sequencing-by-synthesis approach (e.g., Illumina), or single molecule sequencing (e.g., by the single molecule, real-time system from Pacific Biosciences, or by nanopore sequencing (e.g., by Oxford Nanopore Technologies), or using probes, e.g., in hybridization arrays or capture probes. In some embodiments, the sequencing process may be preceded by amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

At step 2308, a first set of the sequence reads is identified. In some embodiments, each sequence read of the first set of the sequence reads includes an ending sequence corresponding to the first sequence end signature. In some embodiments, the first set of sequence reads include ending sequences corresponding to ends of the plurality of cell-free DNA molecules. The ending sequences having the first sequence end signature may be determined using a reference genome, e.g., to identify bases just before a start position or just after an end position. Such bases will still correspond to ends of cell-free DNA fragments, e.g., as they are identified based on the ending sequences of the fragments.

At step 2310, a first amount of the first set of the sequence reads is determined. In some embodiments, the first amount of the first set of the sequence reads may be counted (e.g., stored in an array in memory).

At step 2312, a first parameter is determined using the first amount and potentially another amount of the sequence reads. In some examples, both of such amounts can be separate parameters. As described herein, the other amount can take various forms, e.g., corresponding to a total number of sequence reads and/or DNA molecules analyzed. As another example, the other amount can correspond to an amount of a second set of sequence reads that each include an ending sequence corresponding to one or more other sequence end signatures (end motifs). In some embodiments, the first parameter is a ratio of amounts between two sets of sequence reads having their respective end motifs (e.g., CCCA/AAAA). In some instances, the first parameter (e.g., DNAS1L3/DFFB) is generated by using the first amount of sequence reads that include ending sequences corresponding to an end signature corresponding to the first nuclease (e.g., DNASE1L3) and a second amount of sequence reads that include ending sequences corresponding an end signature of the second nuclease (e.g., DFFB), in which the second nuclease is differentially regulated in an abnormal tissue cells of one or more tissue types relative to a normal tissue of the one or more tissue types. In some instances, the first parameter indicates a motif diversity score, relative frequencies of end motifs, or DNASE1L3/DFFB-cutting signature ratio.

Differences in relative frequencies of end motifs can be detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

In some instances, the same amount of sequence reads is used for normalizing each parameter that represents expression levels of a corresponding nuclease. Additionally or alternatively, different amounts of sequence reads can be used to normalize each parameter for a corresponding nuclease.

At step 2314, the fractional concentration of the clinically-relevant DNA molecules in the biological sample is estimated. Parameters generated based on respective nucleases can be used to determine the fractional concentration of clinically-relevant DNA molecules based on sequence end signatures. These respective parameters can be combined to form a new combined parameter, e.g., as a ratio, a ratio of respective functions of the respective parameters, and as two inputs to more complex functions, such as a machine learning model. Example combined parameters can include DNASE1L3/DFFB, DNASE1/DFFB, or other ratios of DNASE1L3:DNASE1:DFFB. Further, the parameters of more than two nucleases can be used, e.g., relative parameters of 3 or more nucleases can be used.

In some embodiments, the fractional concentration of the clinically-relevant DNA molecules is estimated based on analyzing a set of parameters, in which each parameter corresponds to an amount of sequence reads that each include an ending sequence corresponding to a particular sequence end signature in combination with another amount (e.g., for normalization) of sequence reads. For instance, a parameter can include a particular combination of frequency ratios between two sets of sequence reads with their respective end signatures. For example, a first parameter of the set of parameters may correspond to a ratio of end signatures (e.g., CGTA/GGAG) between a first amount of sequence reads each including an ending sequence corresponding to an end signature of a first nuclease and another amount of sequence reads, and a second parameter of the set of parameters may correspond to a ratio of end signatures (e.g., CCCA/AAAA) between a second amount of sequence reads each including an ending sequence corresponding to an end signature of a second nuclease and a third amount of sequence reads. In some instances, the third amount of sequence reads is the other amount of sequence reads used to determine the first parameter.

In some embodiments, the fractional concentration is estimated by comparing the first parameter to one or more calibration values determined from one or more calibration samples whose fractional concentration of the clinically-relevant DNA molecules are known. For example, the comparison can be whether the first parameter (e.g., CCCA/AAAA end-motif ratio) is higher or lower than the calibration value that represents a particular fractional concentration of clinically-relevant DNA molecules. The comparison can involve comparing to a calibration curve (composed of the calibration data points), and thus the comparison can identify the point on the curve having the first value of the first parameter. The fractional concentration corresponding to the identified point can then be used to estimate the fractional concentration of the first parameter. For example, the first parameter can be provided as an input to the calibration function (e.g., a linear or non-linear fit) to obtain an output of the fractional concentration. A same technique can be used to determine a characteristic value for a target tissue type.

The comparison can be to a plurality of calibration values. The comparison can occur by inputting the first parameter into a calibration function fit to the calibration data that provides a change in the first parameter relative to a change in the fractional concentration of the clinically-relevant DNA in the sample. As another example, the one or more calibration values can correspond to other parameters in the one or more calibration samples. A multidimensional calibration curve can be used. For example, the first parameter and the second parameter can be input into a multi-dimensional calibration function identified from a functional fit (e.g., a calibration surface) of calibration data points from calibration samples, whose fractional concentration is known and that have had the first and second parameter measured.

In various embodiments, measuring a fractional concentration of clinically-relevant DNA can be performed using a tissue-specific allele or epigenetic marker, or using a size of DNA fragments, e.g., as described in US Patent Publication 2013/0237431, which is incorporated by reference in its entirety. Tissue-specific epigenetic markers can include DNA sequences that exhibit tissue-specific DNA methylation patterns in the sample.

In various embodiments, the clinically-relevant DNA can be selected from a group consisting of fetal DNA, tumor DNA, DNA from a transplanted organ, and a particular tissue type (e.g., from a particular organ). The clinically-relevant DNA can be of a particular tissue type, e.g., the particular tissue type is liver or hematopoietic. When the subject is a pregnant female, the clinically-relevant DNA can be placental tissue, which corresponds to fetal DNA. As another example, the clinically-relevant DNA can be tumor DNA derived from an organ that has cancer.

Generally, it is preferred for the one or more calibration values determined from one or more calibration samples to be generated using a similar assay as used for the biological (test) sample for which the fractional concentration is being measured. For example, a sequencing library can be generated in a same manner. Two example processing techniques are GeneRead (www.qiagen.com/us/shop/sequencing/generead-size-selection-kit/#orderinginformation) and SPRI (solid phase reversible immobilization, AMPure bead, www.beckman.hk/reagents_depr/genomic_depr/cleanup-and-size-selection/per). GeneRead can remove the short DNA, which are predominantly tumor fragments, which can affect the relative frequencies of the end motifs for the wildtype and mutant fragments, as well as for the fetal and transplant cases.

C. Characteristic of a Target Tissue

In various embodiments, cell-free DNA end signatures are used to determine a characteristic of a target tissue. For example, the determined characteristic can include a particular gestational age or range (e.g., 8 weeks, 9-12 weeks), e.g., when a nuclease is differentially regulated between fetal tissue and maternal tissue. In another example, the determined characteristic can be a size or nutrition status of an organ corresponding a particular tissue type, which may be affected by metabolic changes of a corresponding subject over the course of pregnancy. At different gestational ages, the metabolism of many organs in both maternal and fetal sides, as well as placenta, would be changed.

1. Determining Gestational Age

DNASE1L3 expression levels can be upregulated in pregnant subjects with late gestational ages (e.g., third trimester), relative to DNASE1L3 expression levels in pregnant subjects with early gestational ages (e.g., first trimester). Thus, one or more end-cutting signatures representing a particular nuclease can be used to determine a parameter that is predictive of a gestational age of a pregnant subject.

FIGS. 24A and 24B show boxplots of DNASE1L3 expression levels across different gestational ages of human placenta tissues (A, DNASE1L3) and murine placenta tissues (B, Dnase113), according to some embodiments. The nuclease activities would vary according to different pathophysiological stages such as pregnancy. For example, we analyzed one microarray-based dataset, from Gene Expression Omnibus (NCBI) (www.ncbi.nlm.nih.gov/geo/query/acc.cgi?acc=GSE28551), comprising 21 women recruited with uncomplicated pregnancies who delivered at term and 16 healthy women undergoing surgical abortion at 9-12 weeks gestation. As shown in FIG. 24A, DNASE1L3 expression levels was found to be significantly increased in the human placenta at the 3^{rd} trimester (median expression level: 12.4; range: 10.9-14.4), in comparison with the 1st trimester (median expression level: 10.3; range: 7.7-12.4) (P value <0.0001, Mann-Whitney U test). On the other hand, we also analyzed another microarray-based dataset from Expression Omnibus (NCBI) (www.ncbi.nlm.nih.gov/geo/query/acc.cgi?acc=GSE41438), comprising 5 mice from each of the gestational ages of days 10, 15 and day 19. The results showed that the orthologous gene DNASE1L3 in mouse was also significantly increased at the advanced gestational ages of day 15 and 19 (median expression level: 10.1; range: 7.8-10.4), compared with the early gestational age of day 10 (median expression level: 8.8; range: 8.5-9.9) (P value=0.02, Mann-Whitney U test) (FIG. 24B).

FIG. 25 shows a boxplot of DNASE1L3/DFFB-cutting signature ratios across different gestational ages according to some embodiments. As shown in FIG. 25, nuclease-cutting signature ratio of CCCA to AAAA end motifs increased as the gestational age progressed. These results suggest that nuclease-cutting signature ratio between two motifs can serve as a biomarker for assessing the gestational ages. These data therefore support the feasibility of using nuclease-cutting signature ratios to reflect pathophysiological changes over time, for example including that for cancer. On the basis of this finding, one would envision that the nuclease-cutting signature ratio would be used for monitoring or predicting the response to therapeutic intervention for patients with cancers or other diseases over time.

2. Methods for Determining Characteristic Value of Target Tissue

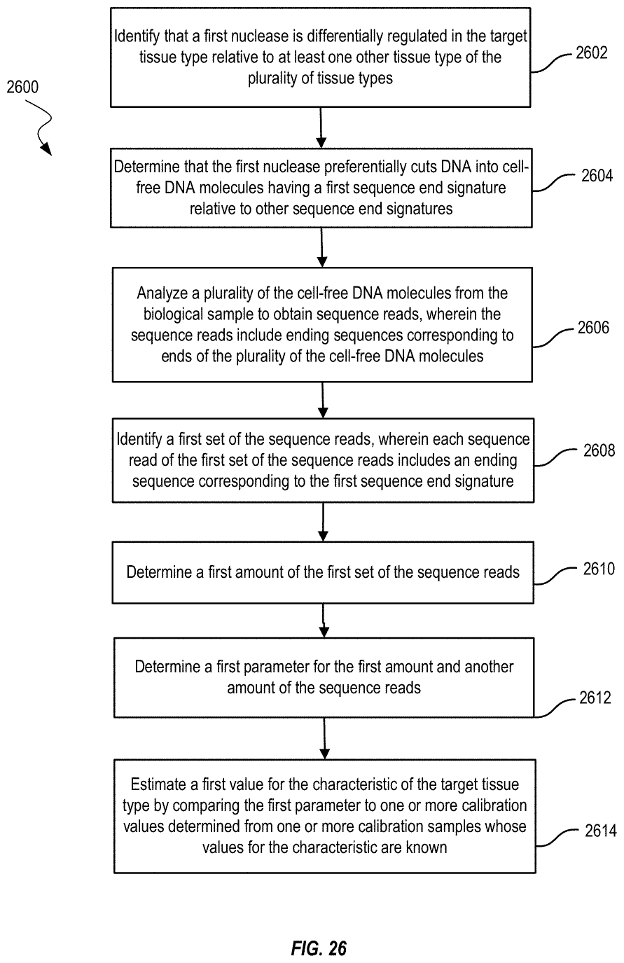

FIG. 26 is a flowchart illustrating a method of determining a characteristic of a target tissue type based on sequence end signatures, according to some embodiments. The characteristic of the target tissue type can be determined by analyzing a biological sample including a mixture of cell-free DNA molecules from a plurality of tissue types. In some embodiments, the characteristic of a target tissue type indicates a gestational age in placental tissues, or conditions relating to the placental tissue including preeclampsia, preterm birth, fetal chromosomal aneuploidies, and/or fetal genetic disorder. The characteristic of the target tissue type may also be used to differentiate tissue types, such as differentiating liver-derived DNA molecules and DNA molecules mainly of hematopoietic origin.

At step 2602, a first nuclease is differentially regulated in a target tissue type relative to at least one other tissue type of the plurality of tissue types is identified. In some embodiments, the clinically-relevant DNA molecules are from the target tissue type. In some instances, a second nuclease being differentially regulated in the target tissue type of one or more tissue types relative to at least one other tissue type of the plurality of tissue types is also identified.

At step 2604, the first nuclease is determined to preferentially cut DNA into DNA molecules having a first sequence end signature relative to other sequence end signatures. In some instances, the cutting preference of the first nuclease is determined by analyzing a biological sample of another organism (e.g., mice). In some instances, the cutting preference of the first nuclease is determined by using a permutation analysis, so as to determine the combination of end signatures exhibiting the most discriminating power in differentiating tissue DNA molecules (e.g., liver-derived DNA molecules from DNA molecules mainly of hematopoietic origin).

At step 2606, a plurality of the cell-free DNA molecules from the biological sample are analyzed to obtain sequence reads. In some embodiments, the sequence reads include ending sequences corresponding to ends of the plurality of the cell-free DNA molecules. In some embodiments, paired-end sequencing is used to obtain sequence reads, which two sequence reads are obtained from the two ends of a DNA fragment, e.g., 30-120 bases per sequence read. As described herein, sequence read may be obtained in a variety of ways, e.g., using sequencing techniques (e.g., using a sequencing-by-synthesis approach (e.g., Illumina), or single molecule sequencing (e.g., by the single molecule, real-time system from Pacific Biosciences, or by nanopore sequencing (e.g., by Oxford Nanopore Technologies), or using probes, e.g., in hybridization arrays or capture probes. In some embodiments, the sequencing process may be preceded by amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

At step 2608, a first set of the sequence reads is identified. In some embodiments, each sequence read of the first set of the sequence reads includes an ending sequence corresponding to the first sequence end signature. In some embodiments, the first set of sequence reads include ending sequences corresponding to ends of the plurality of cell-free DNA molecules. The ending sequences having the first sequence end signature may be determined using a reference genome, e.g., to identify bases just before a start position or just after an end position. Such bases will still correspond to ends of cell-free DNA fragments, e.g., as they are identified based on the ending sequences of the fragments.

At step 2610, a first amount of the first set of the sequence reads is determined. In some embodiments, the first amount of the first set of the sequence reads may be counted (e.g., stored in an array in memory).

At step 2612, a first parameter is determined using the first amount and potentially another amount of the sequence reads. In some examples, both of such amounts can be separate parameters. The other amount can take various forms, e.g., corresponding to a total number of sequence reads and/or DNA molecules analyzed. As another example, the other amount can correspond to an amount of a second set of sequence reads that each include an ending sequence corresponding to one or more other sequence end signatures (end motifs). The first parameter can be a ratio of amounts between two sets of sequence reads having their respective end motifs (e.g., CCCA/AAAA).

In some instances, the first parameter (e.g., DNASE1L3/DFFB) is generated by using the first amount of sequence reads that include ending sequences corresponding to an end signature of the first nuclease (e.g., DNASE1L3) and a second amount of sequence reads that include ending sequences corresponding to an end signature of the second nuclease (e.g., DFFB), in which the second nuclease is differentially regulated in an abnormal tissue cells of one or more tissue types relative to a normal tissue of the one or more tissue types. In some instances, the first parameter indicates a motif diversity score, relative frequencies of end motifs, or DNASE1L3/DFFB-cutting signature ratio.

Differences in relative frequencies of end motifs can be detected for different types of tissue and for different phenotypes, e.g., different levels of pathology. The differences can be quantified by an amount of DNA fragments having specific end motifs or an overall pattern, e.g., a variance (such as entropy, also called a motif diversity score), across a set of end motifs (e.g., all possible combinations of the k-mers corresponding to the length used).

In some instances, the same amount of sequence reads is used for normalizing each parameter that represents expression levels of a corresponding nuclease. Additionally or alternatively, different amounts of sequence reads can be used to normalize each parameter for a corresponding nuclease.

At step 2614, a first value for the characteristic of the target tissue type is estimated by comparing the first parameter to one or more calibration values determined from one or more calibration samples whose values for the characteristic are known. Step 2614 may be performed in a similar manner as step 2314 of FIG. 23.

Parameters generated based on respective nucleases can thus be used to determine the characteristic of the target tissue type. These respective parameters can be combined to form a new combined parameter, e.g., as a ratio, a ratio of respective functions of the respective parameters, and as two inputs to more complex functions, such as a machine learning model. Example combined parameters can include DNASE1L3/DFFB, DNASE1/DFFB, or other ratios of DNASE1L3:DNASE1:DFFB. Further, the parameters of more than two nucleases can be used, e.g., relative parameters of 3 or more nucleases can be used.

In some embodiments, the first value for the characteristic of the target tissue type is estimated based on analyzing a set of parameters, in which each parameter corresponds to an amount of sequence reads that each include an ending sequence corresponding to a particular sequence end signature in combination with another amount (e.g., for normalization). For instance, a parameter can include a particular combination of frequency ratios between two sets of sequence reads with their respective end signatures. For example, a first parameter of the set of parameters may correspond to a ratio of end signatures (e.g., CGTA/GGAG) between a first amount of sequence reads each including an ending sequence corresponding to an end signature of a first nuclease and another amount of sequence reads, and a second parameter of the set of parameters may correspond to a ratio of end signatures (e.g., CCCA/AAAA) between a second amount of sequence reads each including an ending sequence corresponding to an end signature of a second nuclease and a third amount of sequence reads. In some instances, the third amount of sequence reads is the other amount of sequence reads used to determine the first parameter.

The determined characteristic can include a gestational age or range (e.g., 8 weeks, or 9-12 weeks), e.g., when a nuclease is differentially regulated between fetal tissue and maternal tissue. In another example, the determined characteristic can be a particular tissue type (e.g., liver cells) relative to the other tissue type (e.g., hematopoietic cells). The characteristic of the target tissue type may also indicate a particular condition of the target tissue type (e.g., HCC, preeclampsia, preterm birth). In another example, the determined characteristic can be a size or nutrition status of an organ corresponding a particular tissue type (e.g., liver cells).

The comparison can be to a plurality of calibration values. The comparison can occur by inputting the first parameter into a calibration function fit to the calibration data that provides a change in the first parameter relative to a change in the characteristics in the sample. As another example, the one or more calibration values can correspond to other parameters in the one or more calibration samples.

Generally, it is preferred for the one or more calibration values determined from one or more calibration samples to be generated using a similar assay as used for the biological (test) sample. For example, a sequencing library can be generated in a same manner. Two example processing techniques are GeneRead (www.qiagen.com/us/shop/sequencing/generead-size-selection-kit/#orderinginformation) and SPRI (solid phase reversible immobilization, AMPure bead, www.beckman.hk/reagents_depr/genomic_depr/cleanup-and-size-selection/per). GeneRead can remove the short DNA, which are predominantly tumor fragments, which can affect the relative frequencies of the end motifs for the wildtype and mutant fragments, as well as for the fetal and transplant cases.

### V. Jagged-End Analysis Based on Differential Regulation of Nucleases

As described herein, one could determine if a plasma DNA carries a single-stranded end, termed jagged ends, by taking advantage of unmethylated cytosines or methylated cytosines in the DNA end repair step. The DNA end repair would fill in the single-stranded DNA to form double-stranded DNA. For a method based on the DNA end repair involving the filling of unmethylated cytosines, the degree of jaggedness could be deduced by the reduction of methylation level in the read 2. Such a degree of jaggedness inferred by the filling of unmethylated cytosines was referred to JI-U. On the other hand, for a method based on the end repair involving the filling of methylated cytosines, the degree of jaggedness could be deduced by the increase of methylation level in the read 2. Such a degree of jaggedness inferred by the filling of methylated cytosines was referred to JI-M.

In some embodiments, different reference values can be determined, such that they are compared with the jaggedness index value to differentiate abnormal tissues from normal tissues, determine fractional concentration of clinically-relevant DNA, differentiate tissue types, and the like. For example, the reference value can change based on whether the nuclease is upregulated or downregulated, in combination with whether the nuclease causes jaggedness to increase/decrease relative to a typical/normal level of jaggedness in a cell-free sample.

In other embodiments, multiple jaggedness index values can be generated to represent expression levels corresponding to different nucleases. For example, a first nuclease can be associated with an end signature that results in a first length of overhang between the two DNA strands. A second nuclease can be associated with a different end signature that results in a second length of overhang between the two DNA strands.

The reference value can vary based on the first and second length relative to a typical/normal value, and vary based on whether the nucleases are upregulated or downregulated. For instance, a larger deviation from normal would be expected for two nucleases that are both upregulated/downregulated and both result in shorter/longer lengths than normal. Or a smaller deviation can be expected if the nucleases act in different direction for the jaggedness index value. The multiple jaggedness index values can be compared to respective reference values, so as to differentiate abnormal tissues from normal tissues, determine fractional concentration of clinically-relevant DNA, differentiate tissue types, and the like. For example, the multiple jaggedness index values of nucleases (e.g., DNASE1L3, DFFB, and DNASE1) are plotted in a three-dimensional scatter plot, such that a hyperplane can be determined for differentiating abnormal and normal tissues.

A. Jaggedness of Cell-Free DNA Across Various Nucleases and Fragment Sizes

Although the jaggedness of cell-free DNA molecules with a size of between 130 to 160 bp was increased in mice with the DNASE1L3 deletion (Jiang et al. Genome Res. 2020; 30:1144-1153) compared with wild-type mice, other fragment sizes can be considered for jagged-end analysis for some nucleases (e.g., DNASE1L3). For illustrative purposes, jaggedness of cell-free DNA are assessed with a wide range size from 50 to 600 bp. Jaggedness of cell-free DNA was defined by methylation level reduction at CpG sites in read 2 compared with read 1, on the basis of massively parallel bisulfite sequencing. The principles of the quantification of jaggedness of cell-free DNA were described herein, and in U.S. Application No. 63/122,669, filed Dec. 8, 2020, and U.S. Application No. 63/193,508, filed May 26, 2021, the entire contents of which are incorporated herein by reference in its entirety and for all purposes.

1. DNASE1L3

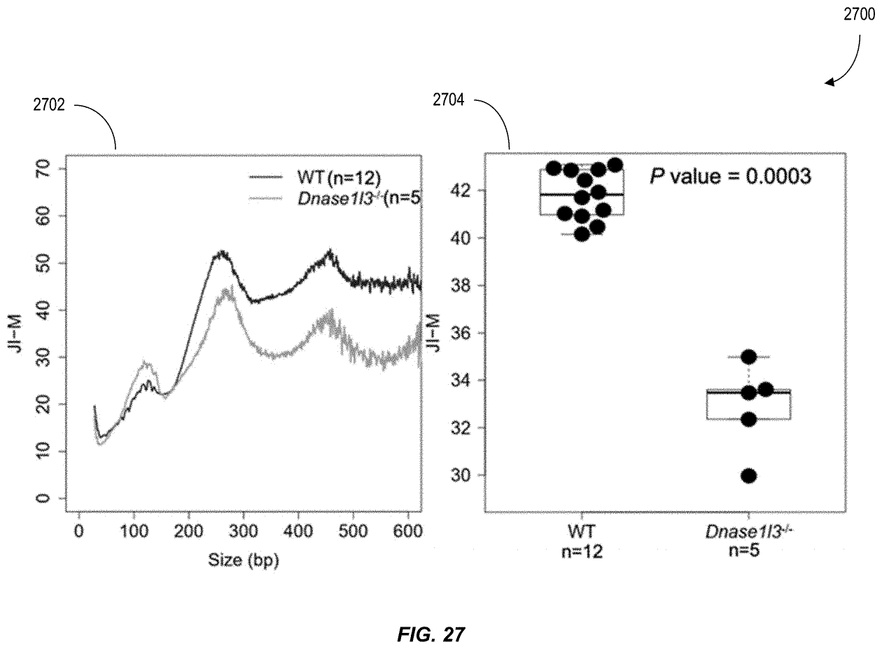

FIG. 27 shows a set of graphs 2700 that show jaggedness of plasma DNA between wild-type mice and mice with DNASE1L3 deletion. In FIG. 27, graph 2702 shows JI-M values across various fragment sizes for wildtype mice and mice with deletion of DNASE1L3. Box plot 2704 shows for JI-M values of plasma DNA within the 200 to 600 bp range for wildtype mice and mice with deletion of DNASE1L3. In this example, we measured the jaggedness index in a wider range size from 50 to 600 bp for wild-type (n=12) and DNASE1L3^{-/-} mice (n=5) with the use of methylated cytosines. The median number of mapped paired-end reads was 115 million (range: 51-216 million). As shown in the graph 2702, in addition to the jaggedness for plasma DNA molecules with the size between 130 to 160 bp being higher in plasma of mice with the DNASE1L3 deletion than wild-type mice, the jaggedness of plasma DNA were shown to be lower for those molecules greater than 200 bp in mice with the DNASE1L3 deletion.

As shown in the graph 2702, a biphasic jaggedness distribution across fragment size was observed in mice with deletion of DNASE1L3 compared with wild-type mice. In short fragments with size shorter than 170 bp, which is nearly the size of one nucleosome, an increase of jaggedness can be seen in DNASE1L3″ mice. In contrast, the box plot 2704 shows that, while in fragments longer than 200 bp, a median of 24.95% decrease can be observed in DNASE1L3′ mice.

In some instances, the use of jaggedness of plasma DNA molecules greater than 200 bp leads to a larger difference between mice with and without deletion of DNASE1L3 (the box plot 2704), compared with the results based on plasma DNA molecules ranged from 130 to 160 bp. These results indicate that the use of jaggedness of relatively longer plasma DNA would reflect the DNA nuclease activity. In some embodiments, jaggedness of plasma DNA is determined based on DNA molecules having a size greater than, but not limited to, 170 bp, 180 bp, 190 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 400 bp, 450 bp, 500 bp, 550 bp, 600 bp or others.

2. DNASE1

The increase of jaggedness exists in short fragments (e.g., <170 bp) in DNASE1L3^{-/-} mouse model could be attributed to other responsible enzymes. For instance, we tested the impact of DNASE1 on plasma DNA jagged ends.

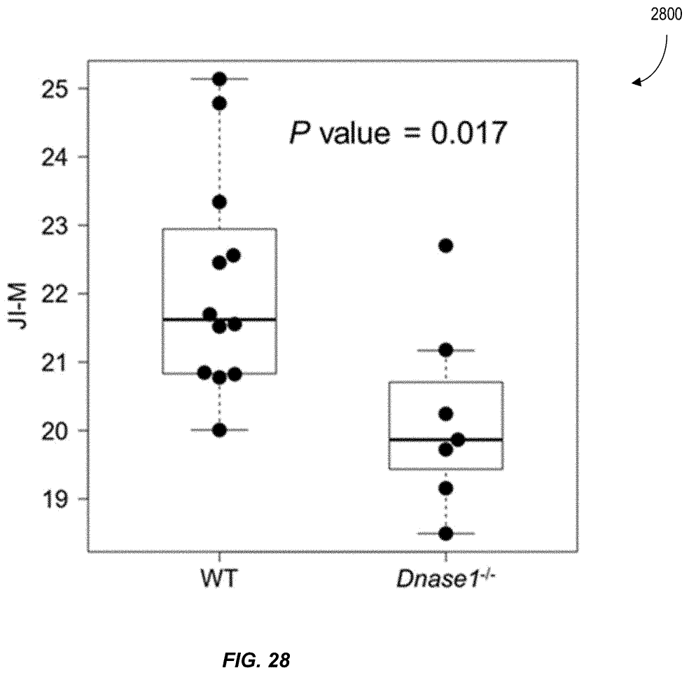

FIG. 28. shows a box plot that identifies jaggedness of plasma DNA (JI-M) between Dnase1^{-/-} mice and WT mice. In FIG. 28, a set of 7 DNASE1^{-/-} mice and 12 WT mice were used to explore the difference in jaggedness. In this example, the jaggedness index was measured for DNA fragments having a size that is less than 170 bps. The average of jaggedness presents in the DNASE1^{-/-} mice DNA molecules (mean JI-M value: 20.19; range: 18.49-22.70) were significantly lower than those from molecules from WT mice (mean JI-M value: 22.12; range: 20.01-25.14; P-value=0.017, Mann-Whitney U test). This result indicates that the DNASE1 would be one of factors that can introduce jagged ends in cell-free DNA molecules.

3. DFFB

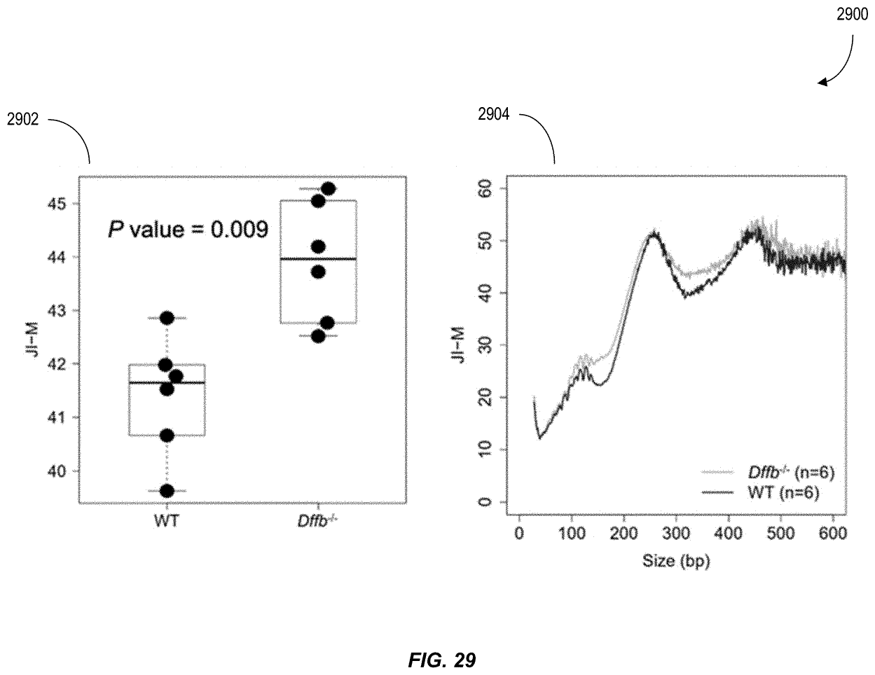

To further investigate jagged end generation related enzymes, we took use of 6 Dff^{-/-} mice and 6 WT mice. FIG. 29 shows a set of graphs that identify jaggedness of plasma DNA between WT and DFFB^{-/-} mice. In FIG. 29, box plot 2902, shows difference of JI-M values between WT and DFFB^{-/-} mice. The knockout of DFFB (median JI-M value: 43.96; range: 42.53-45.28) leads to a 5.57% increase of JI-M with fragment size longer than 200 bp compared with WT mice (median JI-M value: 41.64; range: 39.63-42.86; P-value=0.009, Mann-Whitney U test). In addition, graph 2904 shows JI-M values of plasma DNA across different fragment sizes between WT and DFFB^{-/-} mice. As shown in the graph 2904, increase of JI-M values can also be seen in JI-M distribution across different fragment sizes. This result can preliminarily reveal that DFFB might facilitate the generation of very short jagged ends or blunt ends during DNA fragmentation process.

These results demonstrates that the use of jagged ends of plasma DNA across different sizes could inform various DNA nuclease activities. The diseases associated with aberrations in DNA nuclease activities would be detected through the analysis of jagged ends of plasma DNA according to embodiments present in this disclosure.

B. Fractional Concentration of Clinically-Relevant DNA

In some embodiments, a specified length of overhang between two DNA strands can be associated with an end-cutting signature of a particular nuclease.

For a biological sample of a particular subject, a parameter that identifies an amount of DNA molecules having this property (e.g., the specified length of overhang) can be generated, and the parameter can be used to determine fractional concentration of clinically-relevant DNA for the subject. For example, a parameter such as jaggedness index value can be indicative of a biological sample including a particular amount of fetal-specific DNA, tumor DNA, or transplanted DNA. For example, a determination that the jaggedness index value is higher relative to another jaggedness index value of another sample indicates a different fractional concentration of fetal-specific DNA or tumor DNA.

1. Jaggedness for Fetal and Maternal DNA

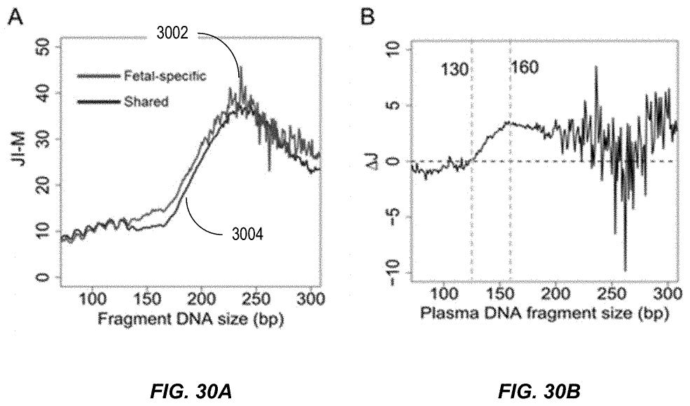

FIGS. 30A and 30B shows comparisons of jaggedness index values between fetal-specific and shared DNA molecules, according to some embodiments. As presented in fetal-specific data 3002, higher JI-M values were present in fetal-specific DNA molecules compared with shared DNA fragments represented by shared data 3004, carrying alleles shared between fetal and maternal genotypes (mainly of maternal origin), across the different sizes of plasma DNA fragments (FIG. 30A). FIG. 30B shows the plot of the difference in JI-M (i.e. ΔJ), across different sizes from short to long molecules, between the fetal and maternal DNA molecules in relation to the different sizes of plasma DNA fragments. A positive JI-M means that molecules carrying fetal-specific alleles have higher JI-M. The positive and gradually rising values of ΔJ within the size range of 130 bp to 160 bp were present in fetal-specific DNA across this size range, attaining the maximal value of the range at 160 bp (FIG. 30B).

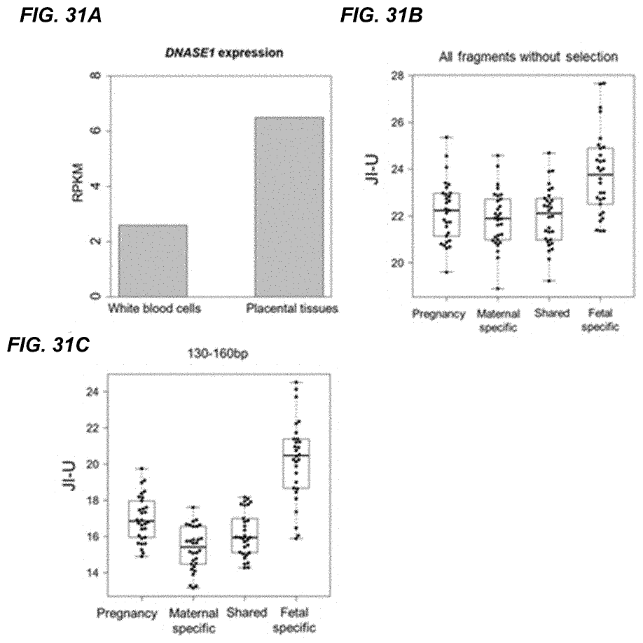

FIG. 31A shows gene expression of DNASE1 in placental tissues and white blood cells, FIG. 31B shows a boxplot of unmethylated-jaggedness index (JI-U) values between fetal-specific and shared fragments without size selection, and FIG. 31C shows a boxplot of JI-U values between fetal-specific and shared fragments within a size range of 130 to 160 bp, according to some embodiments. We found that DNASE1 expression level was 2.5 times higher in placental tissue compared with the DNASE1 expression level of white blood cells. Thus, DNASE1 might be one enzyme which was contributing towards the enhanced jaggedness in fetal DNA molecules (FIG. 31A). We also analyzed 30 pregnant subjects based on JI-U measurement using the previously published dataset (Jiang et al. Clin Chem. 2017; 63:606-608). Compared with JI-U values of shared DNA fragments without size selection (FIG. 31B) (mean: 16.1; range: 14.3-18.2), a higher JI-U values were observed in fetal DNA molecules between 130 and 160 bp (mean: 20.4; range: 15.9-26.2) (FIG. 31C) (P values <0.0001, Mann Whitney U test). The median absolute difference in JI-U between fetal and shared fragments (4.5) was much higher in such a size range of 130 to 160 bp than that of all fragments without size selection (1.7) (P values <0.0001, Mann Whitney U test).

These results suggest that the jaggedness would be informative in reflecting the DNASE1 activity in placental tissues, thus providing a new approach to inform the tissue of origin of plasma DNA molecule. For example, the higher the jaggedness of plasma DNA in a pregnant woman, the more the DNA molecules would be originated from placental tissues. The size selection would enhance the signal to noise ratio in differentiating fetal and maternal DNA molecules.

2. Jaggedness Between Tumor and Non-Tumor DNA

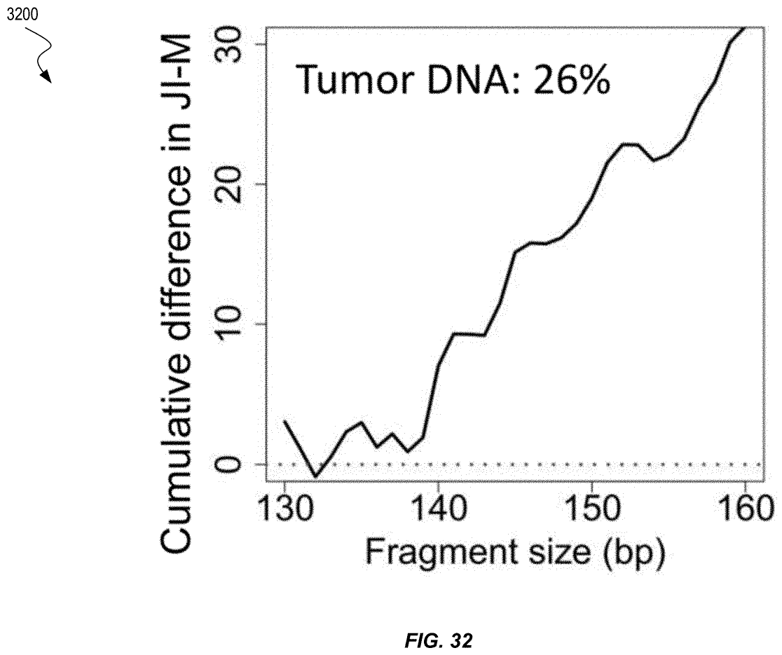

FIG. 32 shows a graph 3200 that identifies a cumulative difference in JI-M values between plasma DNA molecules carrying mutant (tumoral DNA) and wild-type alleles (mainly non-tumoral DNA) in a subject with HCC. As shown in FIG. 32, the plasma DNA carrying the mutant alleles was of tumoral origin, whereas the plasma DNA carrying the wild-type alleles was mainly non-tumoral. There were 31,234 tumor-derived DNA molecules and 209,027 DNA molecules carrying wild-type alleles. The jaggedness of tumor-derived DNA was observed to be higher than that of sequences carrying wild-type, and the cumulative difference in JI-M between the tumor-derived DNA molecules and wild-type molecules increased as the size of DNA fragments increased. This difference in jaggedness can be used to determine a fractional concentration of tumor DNA in a similar manner as for fetal DNA.

3. Methods for Determining Fraction of Clinically-Relevant DNA

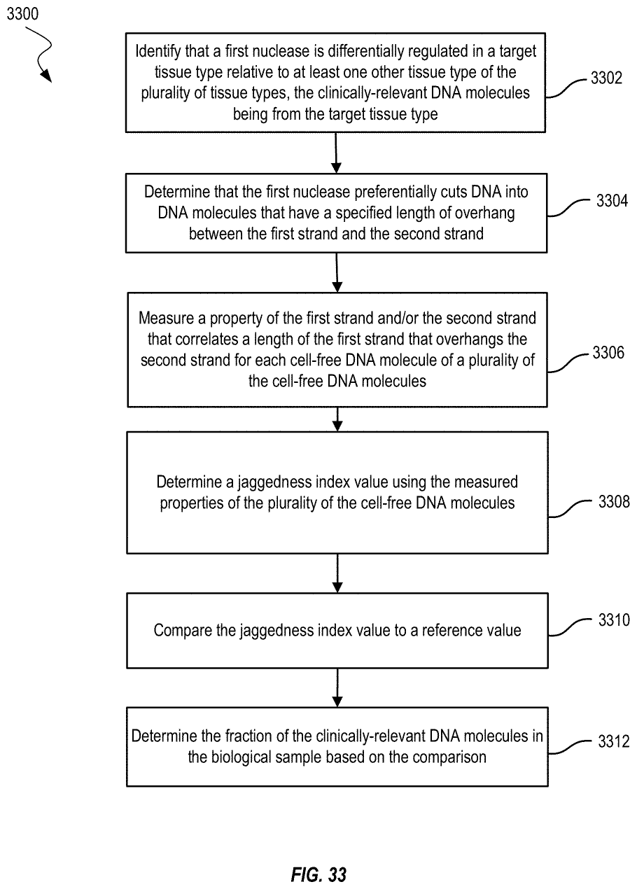

FIG. 33 is a flowchart illustrating a method of determining a fraction of clinically-relevant DNA molecules based on jaggedness index values according to some embodiments. The biological sample may include a mixture of cell-free DNA molecules from a plurality of tissue types, in which each of the cell-free DNA molecules is partially or completely double-stranded with a first strand having a first portion and a second strand. In some instances. the first portion of the first strand of at least some of the cell-free DNA molecules has no complementary portion from the second strand, is not hybridized to the second strand, and is at a first end of the first strand.

At step 3302, a first nuclease is identified as differentially regulated in a target tissue type relative to at least one other tissue type of the plurality of tissue types. The clinically-relevant DNA molecules can be from the target tissue type. For example, DNASE1 expression is relatively upregulated in placental tissue compared with the DNASE1 expression level of white blood cells (FIG. 31A). In another example, DNASE1L3 expression is relatively downregulated in HCC cells compared with liver tissues in healthy subjects. Step 3302 may be performed in a similar manner as step 1702 of FIG. 17.

In some embodiments, multiple jaggedness index values are generated to represent expression levels corresponding to different nucleases. The multiple jaggedness index values can be compared to differentiate abnormal tissues from normal tissues, determine fractional concentration of clinically-relevant DNA, differentiate tissue types, and the like. For example, the multiple jaggedness index values of nucleases (e.g., DNASE1L3, DFFB, and DNASE1) are plotted in a three-dimensional scatter plot, such that a hyperplane can be determined for determining the clinically-relevant DNA molecules.

At step 3304, the first nuclease is determined to preferentially cut DNA into DNA molecules that have a specified length of overhang between the first strand and the second strand. In some instances, the cutting preference of the first nuclease is determined by analyzing a biological sample of another organism (e.g., mice).

At step 3306, a property of the first strand and/or the second strand that correlates a length of the first strand that overhangs the second strand is measured for each cell-free DNA molecule of a plurality of the cell-free DNA molecules. For example, a measured property includes a higher methylation level of the first strand, in which the higher methylation level is correlated with a longer length of the first strand that overhangs the second strand. In another example, a measured property includes a lower methylation level of the first strand, in which the lower methylation level is correlated with a longer length of the first strand that overhangs the second strand. In some instances, the property is a methylation status at one or more sites at end portions of the first strands and/or second strands of each of the plurality of nucleic acid molecules. In other instances, the property is a length of the first strand and/or the second strand that is proportional to the length of the first strand that overhangs the second strand.

In several embodiments, the plurality of the cell-free DNA molecules (for which the property is measured) is configured to have a size within a specified range, e.g., 130 to 160 bps. Other size ranges, including but not limited to, 100-130 bp, 110-140 bp, 120-150 bp, 140-170 bp, 150-180 bp, 160-190 bp, 170-200 bp, 180-210 bp, 190-220 bp, and other size ranges or multiple combinations of different size ranges, would be used in other embodiments.

In some embodiments, jagged ends across different size ranges and different genomic locations can be used as training data for machine learning algorithms to determine fractional concentration of clinically-relevant DNA, differentiate abnormal cells from normal tissue, and the link. The machine learning algorithms may include, but not limited to, linear regression, logistic regression, deep recurrent neural network, Bayes classifier, hidden Markov model (HMM), linear discriminant analysis (LDA), k-means clustering, density-based spatial clustering of applications with noise (DBSCAN), random forest algorithm, and support vector machine (SVM).

At step 3308, a jaggedness index value is determined using the measured properties of the plurality of the cell-free DNA molecules. In some embodiments, the jaggedness index value provides a collective measure that a strand overhangs another strand in the plurality of the cell-free DNA molecules. In some instances, the jaggedness index value identifies a methylation level over the plurality of nucleic acid molecules at one or more sites of end portions of the first strands and/or second strands. In some embodiments, the jaggedness index value corresponds to the measured properties of the plurality of the cell-free DNA molecules having size within a specified range, e.g., 130 to 160 bps (FIG. 31C).

If the first plurality of nucleic acid molecules are in a specified size range, methods may include measuring the property of each nucleic acid molecule of a second plurality of nucleic acid molecules. The second plurality of nucleic acid molecules may have sizes with a second specified size range. Determining the jaggedness index value may include calculating a ratio using the measured properties of the first plurality of nucleic acid molecules and the measured properties of the second plurality of nucleic acid molecules. The jaggedness index value may include the jagged end ratio or the overhang index ratio described herein.

At step 3310, the jaggedness index value is compared to a reference value. The reference value can be determined based on the specified length of overhang between the first strand and the second strand. In some instances, the reference value or the comparison is determined using machine learning with training data sets. The comparison may be used to determine different information regarding the biological sample or the individual.

At step 3312, the fraction of the clinically-relevant DNA molecules in the biological sample is determined based on the comparison. In some instances, the reference value is determined using one or more reference samples of subjects that have the condition. As another example, the reference value is determined using one or more reference samples of subjects that do not have the condition. Multiple reference values can be determined from the reference samples, potentially with the different reference values distinguishing between different levels of the condition.

In various embodiments, measuring a fractional concentration of clinically-relevant DNA can be performed using a tissue-specific allele or epigenetic marker, or using a size of DNA fragments, e.g., as described in US Patent Publication 2013/0237431, which is incorporated by reference in its entirety. Tissue-specific epigenetic markers can include DNA sequences that exhibit tissue-specific DNA methylation patterns in the sample.

In various embodiments, the clinically-relevant DNA can be selected from a group consisting of fetal DNA, tumor DNA, DNA from a transplanted organ, and a particular tissue type (e.g., from a particular organ). The clinically-relevant DNA can be of a particular tissue type, e.g., the particular tissue type is liver or hematopoietic. When the subject is a pregnant female, the clinically-relevant DNA can be placental tissue, which corresponds to fetal DNA. As another example, the clinically-relevant DNA can be tumor DNA derived from an organ that has cancer.

Generally, it is preferred for the one or more calibration values determined from one or more calibration samples to be generated using a similar assay as used for the biological (test) sample for which the fractional concentration is being measured. For example, a sequencing library can be generated in a same manner. Two example processing techniques are GeneRead (www.qiagen.com/us/shop/sequencing/generead-size-selection-kit/#orderinginformation) and SPRI (solid phase reversible immobilization, AMPure bead, www.beckman.hk/reagents_depr/genomic_depr/cleanup-and-size-selection/per). GeneRead can remove the short DNA, which are predominantly tumor fragments, which can affect the relative frequencies of the end motifs for the wildtype and mutant fragments, as well as for the fetal and transplant cases.

The reference value can be a calibration value determined using calibration (reference) samples, which have known classifications and can be analyzed collectively to determine a reference value or calibration function (e.g., when the classifications are continuous variables). Calibration data points for determining the reference value can include a measured jaggedness index value and a measured/known fraction of the clinically-relevant DNA. The measured jaggedness index value for any sample whose fraction is measured via another technique (e.g., using a tissue-specific allele) can be correspond to a reference value. As another example, a calibration curve (function) can be fit to the calibration data points, and the reference value can correspond to a point on the calibration curve. Thus, a measured jaggedness index value of a new sample can be input into the calibration function, which can output the faction of the clinically-relevant DNA.

C. Detecting Abnormal Cells Using Biological Mixture

A specified length of overhang between two DNA strands can also be associated with an end-cutting signature of a particular nuclease. For a biological sample of a particular subject, a parameter that identifies an amount of DNA molecules having this property (e.g., the specified length of overhang) can be used to differentiate abnormal cells from normal cells. For example, a parameter such as jaggedness index value can be predictive of a biological sample including HCC cells, in response to a determination that the jaggedness index value is higher relative to another jaggedness index value that represents normal cells. Such differentiation can be used to predict a level of pathology of the subject.

1. Jaggedness for DNA from Abnormal Vs Normal Cells

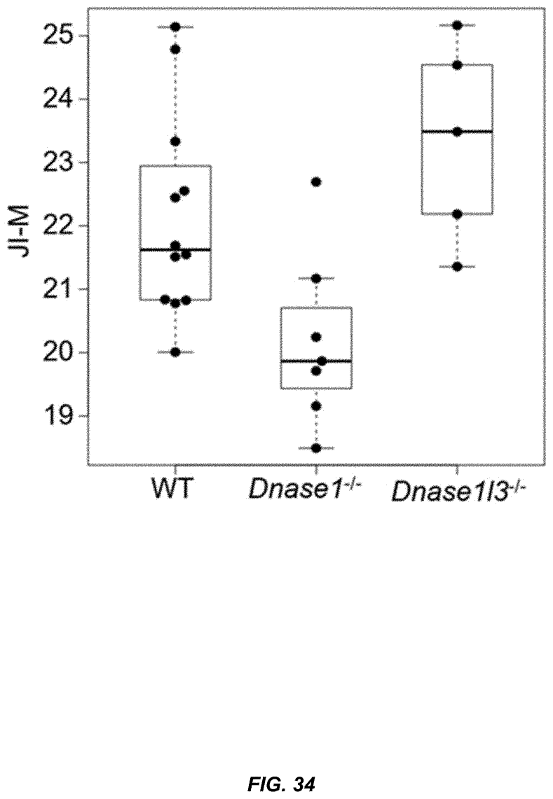

FIG. 34 shows a boxplot of jaggedness index values of plasma DNA in mice across different genotypes including wildtype, DNASE1^{-/-} and DNASE1L3^{-/-}, according to some embodiments. Referring to FIG. 34, the y-axis indicates the jaggedness index value based on the filling of methylated cytosine (JI-M). WT: wildtype; DNASE1^{-/-}: mice with deletion of DNASE1. DNASE1^{-/-}: mice with deletion of DNASE1L3. To further verify the approaches to reveal the link between nucleases and plasma DNA fragmentation patterns, we sequenced 12 wildtype mice, 7 mice with the deletion of DNASE1 (DNASE1^{-/-}) and 5 mice with the deletion of DNASE1L3 (DNASE1L3^{-/-}), with a median of 115 million mapped paired-end reads (range: 31-223 million). We analyzed plasma DNA fragments between 130 and 160 bp. As shown in FIG. 34, an increase of jaggedness (JI-M) was observed in mice with the deletion of DNASE1L3 (DNASE1L3^{-/-}) compared with wildtype mice, whereas a decreasing trend was seen in mice with deletion of DNASE1 (DNASE1^{-/-}) (FIG. 34) (P value: 0.01; Kruskal-Wallis test). These results suggested the possibility of using the jaggedness of plasma DNA to monitor the activities of nucleases. On the other hand, these results also suggested that DNASE1 would contribute towards the generation of long jagged ends in plasma DNA, whereas DNASE1L3 would play a role in generating plasma DNA molecules with relatively short jagged ends or blunt ends.

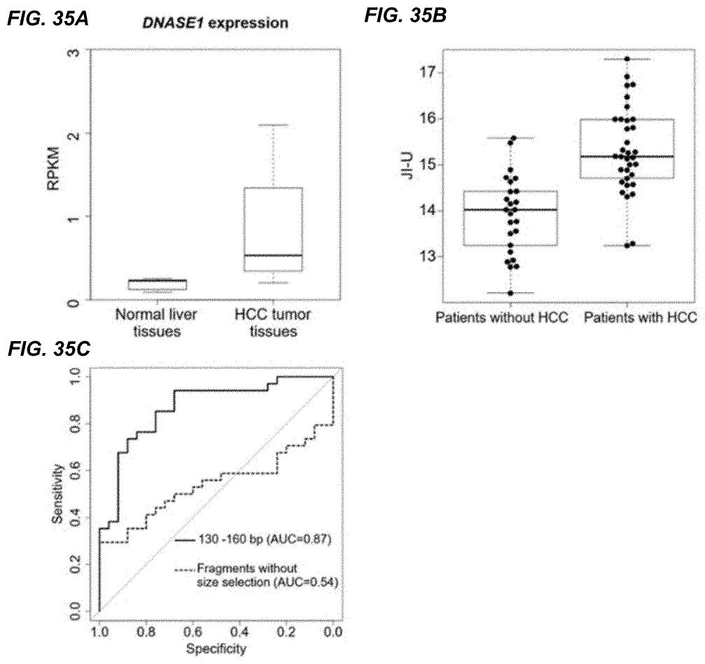

FIG. 35A shows a boxplot of DNASE1 gene expression in normal liver tissues and liver cancer tissues, FIG. 35B shows a boxplot of JI-U values between patients without and with HCC, and FIG. 35C shows ROC curves for comparing performance between JI-U values deduced by fragments with and without size selection, according to some embodiments. On the basis of results shown in mouse models, the aberrations of jaggedness for plasma DNA in patients with HCC would be enhanced, as the DNASE1 expression was upregulated in HCC tumor while the DNASE1L3 was downregulated (FIG. 35A). Much higher JI-U values deduced from fragments within a range of 130 to 160 bp were observed in patients with HCC (mean: 15.3; range: 13.2-17.3) in comparison with patients without HCC (mean: 13.9; range: 12.2-15.6) (FIG. 35B) (P values <0.0001, Mann Whitney U test). AUC of JI-U using fragments between 130 and 160 bp between patients with and without HCC was 0.87, which was superior to the approach without size selection (AUC: 0.54) (FIG. 35C). These results would suggest that in one embodiment, the JI-U for fragments between 130 to 160 bp had the clinical potential for cancer detection. Other size ranges, including but not limited to, 100-130 bp, 110-140 bp, 120-150 bp, 140-170 bp, 150-180 bp, 160-190 bp, 170-200 bp, 180-210 bp, 190-220 bp, and other size ranges or multiple combinations of different size ranges, would be used in other embodiments. In several embodiments, jaggedness index values are generated across different types of tissues to detect tissue abnormalities, including lung cancer, breast cancer, gastric cancer, glioblastoma multiforme, pancreatic cancer, colorectal cancer, nasopharyngeal carcinoma, and/or head and neck squamous cell carcinoma.

In one embodiment, by making use of jagged ends across different size ranges and different genomic locations, machine learning algorithms would be applied to train classifiers for differentiating patients such as cancer, including but not limited to, linear regression, logistic regression, deep recurrent neural network, Bayes classifier, hidden Markov model (HMM), linear discriminant analysis (LDA), k-means clustering, density-based spatial clustering of applications with noise (DBSCAN), random forest algorithm, and support vector machine (SVM).

2. Methods for Determining Abnormality in a Tissue Type

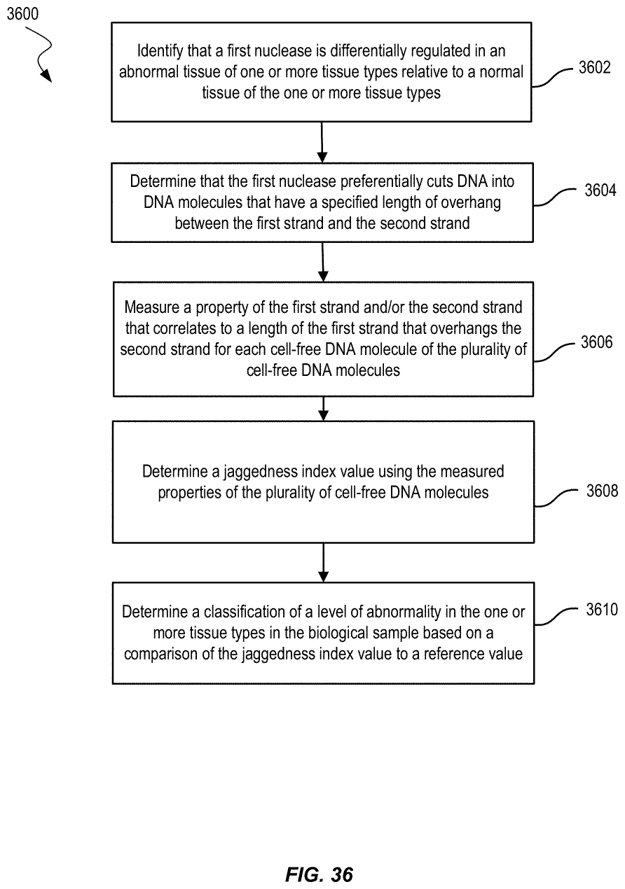

FIG. 36 is a flowchart illustrating a method of classifying a level of abnormality of a tissue based on jaggedness index values, according to some embodiments. The biological sample includes a plurality of cell-free DNA molecules, in which each of the plurality of cell-free DNA molecules is partially or completely double-stranded with a first strand having a first portion and a second strand. In some instances, the first portion of the first strand of at least some of the plurality of cell-free DNA molecules has no complementary portion from the second strand, is not hybridized to the second strand, and is at a first end of the first strand. The abnormality may be a pathology including cancer (e.g., hepatocellular carcinoma, lung cancer, breast cancer, gastric cancer, glioblastoma multiforme, pancreatic cancer, colorectal cancer, nasopharyngeal carcinoma, and/or head and neck squamous cell carcinoma) and an auto-immune disorder (e.g., systemic lupus erythematosus). In some instances, the abnormality in the biological sample is an abnormality of placental tissue (e.g., placental tissue detected in maternal plasma), including preeclampsia, preterm birth, fetal chromosomal aneuploidies, or fetal genetic disorders.

At step 3602, a first nuclease is differentially regulated in abnormal cells of one or more tissue types relative to a normal tissue of the one or more tissue types is identified. For example, DNASE1L3 (Deoxyribonuclease 1 Like 3) expression is relatively downregulated in HCC cells compared with liver tissues in healthy subjects. In another example, DFFB (DNA Fragmentation Factor Subunit Beta) and DNASE1 (Deoxyribonuclease 1) expression are relatively upregulated in in HCC cells compared with liver tissues in healthy subjects. Step 3602 may be performed in a similar manner as step 1702 of FIG. 17.

At step 3604, the first nuclease is determined to preferentially cut DNA into DNA molecules that have a specified length of overhang between the first strand and the second strand. In some instances, the cutting preference of the first nuclease is determined by analyzing a biological sample of another organism (e.g., mice).

In some embodiments, multiple jaggedness index values are generated to represent expression levels corresponding to different nucleases. The multiple jaggedness index values can be compared to differentiate abnormal tissues from normal tissues, determine fractional concentration of clinically-relevant DNA, differentiate tissue types, and the like. For example, the multiple jaggedness index values of nucleases (e.g., DNASE1L3, DFFB, and DNASE1) are plotted in a three-dimensional scatter plot, such that a hyperplane can be determined for differentiating abnormal and normal tissues.

At step 3606, a property of the first strand and/or the second strand that correlates to a length of the first strand that overhangs the second strand is measured for each cell-free DNA molecule of the plurality of cell-free DNA molecules. For example, a measured property includes a higher methylation level of the first strand, in which the higher methylation level is correlated with a longer length of the first strand that overhangs the second strand. In another example, a measured property includes a lower methylation level of the first strand, in which the lower methylation level is correlated with a longer length of the first strand that overhangs the second strand. Step 3606 may be performed in a similar manner as step 3306 of FIG. 33.

At step 3608, a jaggedness index value is determined using the measured properties of the plurality of cell-free DNA molecules. In some embodiments, the jaggedness index value provides a collective measure that a strand overhangs another strand in the plurality of cell-free DNA molecules. In some instances, the jaggedness index value includes a methylation level over the plurality of nucleic acid molecules at one or more sites of end portions of the first strands and/or second strands. In some embodiments, the jaggedness index value corresponds to the measured properties of the plurality of the cell-free DNA molecules having size within a specified range, e.g., 130 to 160 bps (FIG. 35C). Step 3608 may be performed in a similar manner as step 3308 of FIG. 33.

At step 3610, a classification of a level of abnormality in the one or more tissue types in the biological sample is determined based on a comparison of the jaggedness index value to a reference value. The reference value can be determined based on the specified length of overhang between the first strand and the second strand. In some embodiments, the classification of the level of abnormality includes one of a plurality of stages of pathology (e.g., HCC). For example, the aberrations of jaggedness for plasma DNA in patients with HCC would be enhanced, as the DNASE1 expression was upregulated in HCC tumor while the DNASE1L3 was downregulated. In several embodiments, jaggedness index values are generated across different types of tissues to detect tissue abnormalities, including lung cancer, breast cancer, gastric cancer, glioblastoma multiforme, pancreatic cancer, colorectal cancer, nasopharyngeal carcinoma, and/or head and neck squamous cell carcinoma. In some instances, machine learning algorithms are applied to train classifiers for differentiating abnormal cells from normal tissue.

D. Jagged-End Analysis for Determining Genetic Disorders

Autoimmune disease occurs when the body's immune system loses the self-tolerance and mistakenly attacks the cells or tissues of the body itself. Autoimmune disease is a heterogeneous group of diseases, more than 80 types of autoimmune diseases have been identified (Hayter et al. Autoimmunity Reviews. 2012; 11 (10): 754-65; The American Autoimmune Related Diseases Association, Autoimmune Disease List. https://www.aarda.org/diseaselist/). The most common autoimmune diseases include rheumatoid arthritis, type 1 diabetes, multiple sclerosis, systemic lupus erythematosus (SLE), inflammatory bowel disease, psoriasis, scleroderma and autoimmune thyroiditis (Hayter et al. Autoimmunity Reviews. 2012; 11 (10): 754-65).

Autoimmune diseases can affect almost any organ systems. Some of these diseases, such as type 1 diabetes and multiple sclerosis, attack specific organs (Bias et al. Am. J. Hum. Genet. 1986; 39: 584-602) while others, for example SLE, attack multiple organs (Fava et al. Journal of Autoimmunity. 2019; 96: 1-13). The overall cumulative prevalence of all autoimmune diseases is 5% (Hayter et al. Autoimmunity Reviews. 2012; 11 (10): 754-65), but there has been a trend of increasing the prevalence in recent years (Dinse et al. Arthritis & Rheumatology. 2020; 72 (6): 1026-1035). Most autoimmune diseases are chronic and can be controlled with appropriate treatments. However, the vague and variable symptoms between individuals and within individuals over time often make the diagnosis and disease monitoring be difficult.

cfDNA molecules are nonrandomly fragmented and are released from various tissues within body through cell death, such as apoptosis and necrosis (Chandrananda et al. BMC Med Genomics. 2015; 8:29; Thierry et al. Cancer Metastasis Rev. 2016; 35: 347-376). The analysis of plasma nucleic acids has been developing as a non-invasive prognostic and diagnostic tools for various diseases that include but not limit to pregnancy, cancer and allograft rejection (Chiu et al. BMJ. 2011; 342: c7401; Chan et al. N. Engl. J. Med. 2017; 377:513-522; Cohen et al. Science. 2018; 359:926-930; Gielis et al. Am J Transplant. 2015; 15: 2541-2551). High resolution analysis on the genomic and epigenetic signatures of plasma DNA has been shown to reflect disease activities of SLE patients (Chan et al. Proc Natl Acad Sci USA. 2014; 111:E5302-11).

DNA degradation is a critical process for healthy functioning of a body (Keyel. Dev Biol. 2017; 429(1):1-11). Impaired clearance of plasma DNA may cause the development of autoimmunity (Duvvuri et al. Front Immunol. 2019; 10:502). Nucleases, for example the DNase family, play a pivotal role in DNA fragmentation. Different nucleases have different expression in different tissues (The human protein atlas, https://www.proteinatlas.org/). They perform roles in regulating plasma DNA fragmentation (Han et al. Am J Hum Genet. 2020; 106:202-214). A number of studies have demonstrated the involvement of nucleases in the pathogenesis of various autoimmune diseases (Maličlová et al. Autoimmune Dis. 2011; 2011: 945861; Zykova et al. PLoS One; 2010; 5(8):e12096; Gatselis et al. Autoimmunity. 2017 March; 50(2):125-132). Some recent studies have shown the relationship between DNA nucleases and plasma DNA end modalities, such as DNA end motifs (Serpas et al. Proc Natl Acad Sci USA. 2019; 116:641-649; Han et al. Am J Hum Genet. 2020; 106:202-14) and jagged ends (Jiang et al. Genome Res. 2020; 30:1144-1153) in murine model. Such end modalities could be developed as a new type of biomarkers associated with DNA fragmentation. For example, human patients with DNASE1L3 deficiency showed aberrations in fragment sizes and end motifs of plasma DNA (Chan et al. Am J Hum Genet. 2020; 107:882-894).

A number of immunological tests have been developed and routinely used in clinics. For example, a patient's blood sample may be tested for rheumatoid factor (RF), anti-dsDNA antibody, anti-nuclear antibody (ANA), anti-extractable nuclear antigen antibody (ENA), anti-neutrophil cytoplasmic antibody (ANCA), C-reactive protein (CRP) and erythrocyte sedimentation rate (ESR). However, because of the heterogeneity of autoimmune diseases and the importance of early detection and treatment, especially with the fact that most autoimmune diseases are chronic in nature and show vague symptoms, there is a need for sensitive methods for diagnosis and monitoring of autoimmune diseases.

In some embodiments of the present disclosure, various parameters associated with end modalities of cell-free DNA are used for detecting and monitoring autoimmune diseases. The end modalities can include end motifs and jagged ends, and the parameters can include a number of reads (end motifs) and jaggedness index values (jagged ends). Such end modalities can be associated with DNA nuclease activities, including but not limited to DNASE1L3, DFFB, DNASE1, TREX1, AEN, EXO1, DNASE2, ENDOG, APEX1, FEN1, DNASE1L1, DNASE1L2, and EXOG. For example, parameters associated with the presentation of plasma DNA jagged ends can be used to differentiate healthy controls, inactive SLE, and active SLE.

1. Jaggedness of Cell-Free DNA in DNASE1L3 Disease Associated Variants

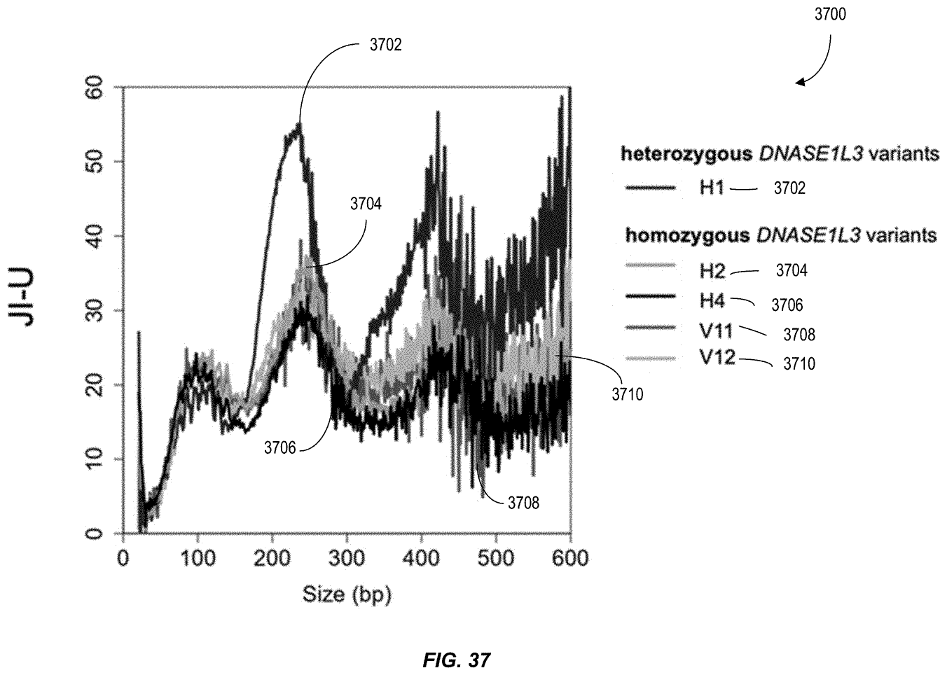

To identify differences of jaggedness in cell-free DNA across DNASE1L3 disease associated variants, jaggedness of plasma DNA was measured for each of 5 human subjects with DNASE1L3 disease associated variants. FIG. 37 shows a graph identifying the distribution of jagged ends in DNA molecules in human subjects with different genotypes of DNASE1L3 associated variants. Line 3702 represents “H1,” which is the heterozygous DNASE1L3 associated variants (i.e., one copy of DNASE1L3 gene being still functional). Line 3704-3710 respectively represent “H2,” “H4,” “V11,” and “V12,” which are subjects with homozygous DNASE1L3 variants (i.e., both copies of DNASE1L3 gene being not able to produce functional DNASE1L3 enzymes). H2 and H4 subjects had homozygous frameshift c.290_291delCA (p.Thr97Ilefs*2) mutation.

In contrast to the JI-U of short plasma DNA fragments (e.g., <150 bp), JI-U of long plasma DNA fragments (e.g., >200 bp) were lower in subjects with homozygous DNASE1L3 associated variants (median JI-U value: 22.01), in comparison with the subject with heterozygous DNASE1L3 variants (median JI-U value: 38.00).

These results suggest that the jaggedness of plasma DNA can be used for detecting the patients with nuclease deficiency. The jaggedness of long plasma DNA would provide a more sensitive approach to reflect the DNA nuclease activity. In one embodiment, the jaggedness of plasma DNA would be used for monitoring therapeutic interventions in the context of the treatment of DNA nuclease associated diseases.

2. Jaggedness of Cell-Free DNA in Subjects with SLE

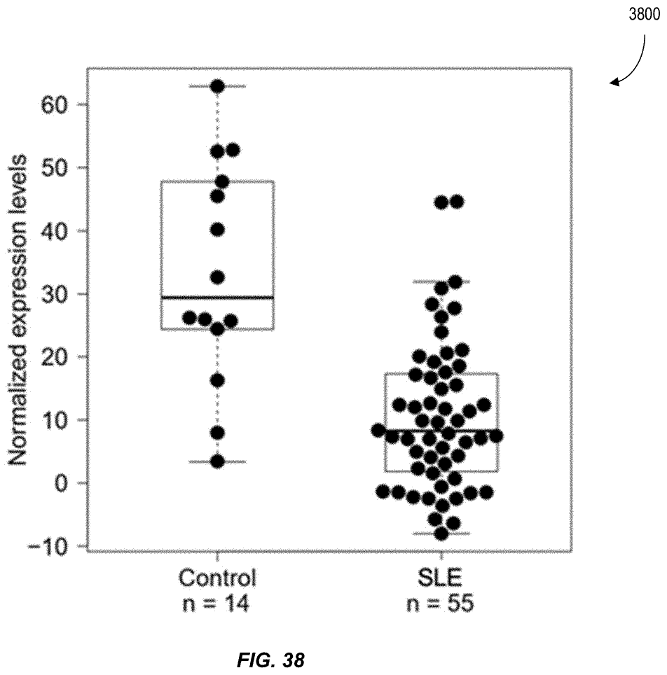

FIG. 38 shows a box plot that identify gene expression level of DNASE1L3 in peripheral blood mononuclear cells between control subjects and patients with SLE. As shown in FIG. 38, a significant reduction of DNASE1L3 expression level was observed in SLE patients from published data (Rinchai D et al. Clin Transl Med. 2020 December; 10(8):e244)(FIG. 3), which can be regarded as DNASE1L3 partial deficiency. In light of the different expression levels of DNASE1L3, we analyzed the jaggedness of plasma DNA based on previously published bisulfate sequencing data, comprising 14 healthy control samples, 14 inactive SLE patients and 20 active SLE patients (Chan et al. Proc Natl Acad Sci USA. 2014; 111:E5302-11).

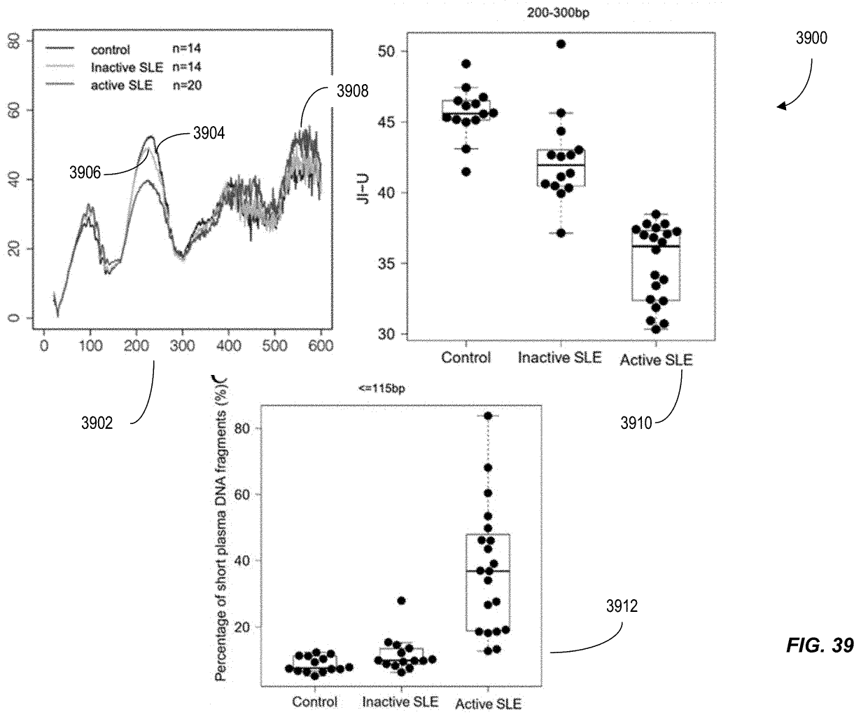

FIG. 39 shows a set of graphs 3900 that identify jaggedness of plasma DNA (JI-U) for control samples, and samples with inactive SLE and active SLE. In FIG. 39, graph 3902 shows jaggedness index (JI-U) values across various DNA fragment sizes in control subjects 3904, subjects with inactive SLE 3906, and subjects with active SLE 3908. The graph 3902 shows that the JI-U of the active SLE patients displayed a lowest jaggedness level for those molecules with around 230 bp in size (median JI-U value: 39.16) compared with those control subjects (median JI-U value: 52.31). The plasma DNA jaggedness of inactive SLE patients (median JI-U value: 48.21) were shown to be in-between the control subjects and patients with active SLE patients.

A box plot 3910 shows jaggedness index values of plasma DNA within the 200 bp-300 bp range for control subjects, subjects with inactive SLE and subjects with active SLE. In the box plot 3910, the jaggedness in selected fragments with a size range between 200 bp to 300 bp allowed us for differentiating three groups, namely, control subjects, subjects with inactive SLE and subjects with active SLE. A median of 25.91% decrease of jaggedness in patients with active SLE (median JI-U value: 36.21; range: 30.34-38.47) was observed relative to control subjects (median JI-U value: 45.59; range: 41.46-49.09) (P-value <0.0001, Mann-Whitney U test), and a median of 8.68% decrease of jaggedness was observed in patients with inactive SLE (median JI-U value: 41.95; range: 37.14-50.51) (P-value=0.00079, Mann-Whitney U test).

As a comparison, a box plot 3912 shows proportion of short plasma DNA (shorter than 115 bp) among control subjects, subjects with inactive SLE and subjects with active SLE. As shown in the box plot 3912, the metric regarding the proportion of short plasma DNA (i.e. <115 bp) (Chan et al. Proc Natl Acad Sci USA. 2014; 111:E5302-11) could only differentiate two groups, namely, subjects with active SLE versus control subjects and subjects with inactive SLE. There was no significant increase observed between inactive SLE and control groups, which shows that jaggedness index values can be a more effective technique for differentiating normal subjects and subjects with SLE.

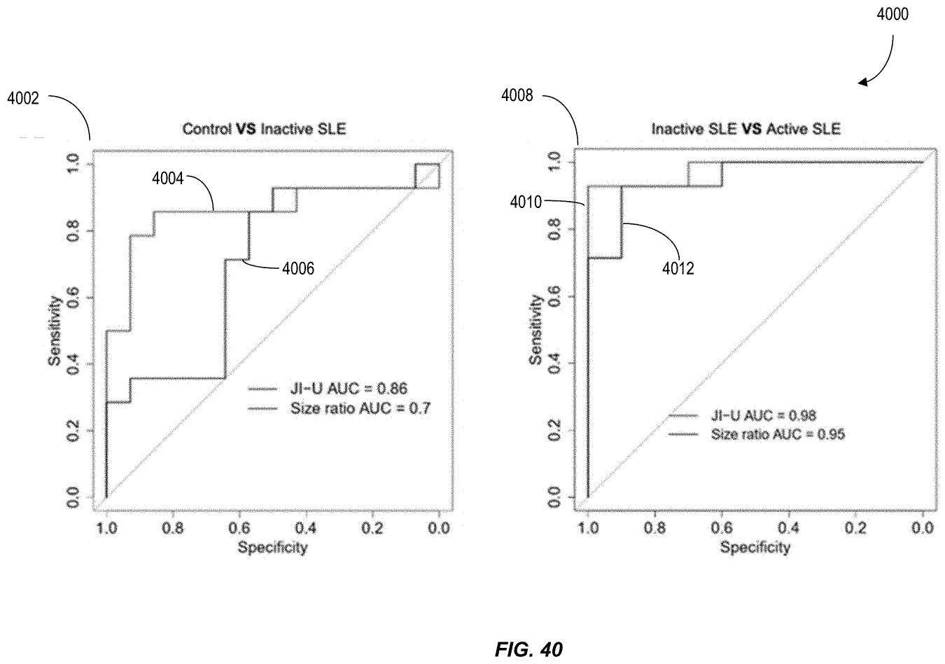

FIG. 40 shows receiver operating characteristic (ROC) curves 4000 that identify performance of jaggedness index values and size ratio methods for differentiating control subjects and SLE subjects. An ROC curve 4002 shows performance of jaggedness index values and size ratio methods for differentiating control subjects and inactive SLE subjects. Compared with the techniques that use plasma DNA size ratio (AUC: 0.7; line 4006), jaggedness index values showed improved performance with AUC of 0.86 in differentiating between patients with inactive SLE and healthy subjects (line 4004). FIG. 40 also shows an ROC curve 4008 that identifies performance of jaggedness index values and size ratio methods for differentiating inactive SLE subjects and active SLE subjects. Here, jaggedness showed an improved performance with AUC of 0.98 (line 4008) in differentiating between patients with active and inactive SLE, compared with the results based on size ratio method (AUC: 0.95; line 4010). Thus, the jaggedness index values determined at a size range of 200 to 300 bp can be used as a biomarker for detecting SLE. In addition, the determination of optimal size ranges for jagged-end analysis can be performed by comparing a reference sample with samples having different nuclease knockouts or samples known to have mutant nuclease genes.

3. Jagged-End Analysis for Samples Incubated with Anticoagulants

Heparin is known to enhance DNASE1 activity and inhibit DNASE1L3 activity. Apart from the use of DNASE1^{-/-} mouse model, we used in-vitro heparin incubation method to further explore the role DNASE1 playing in jagged end generation process.

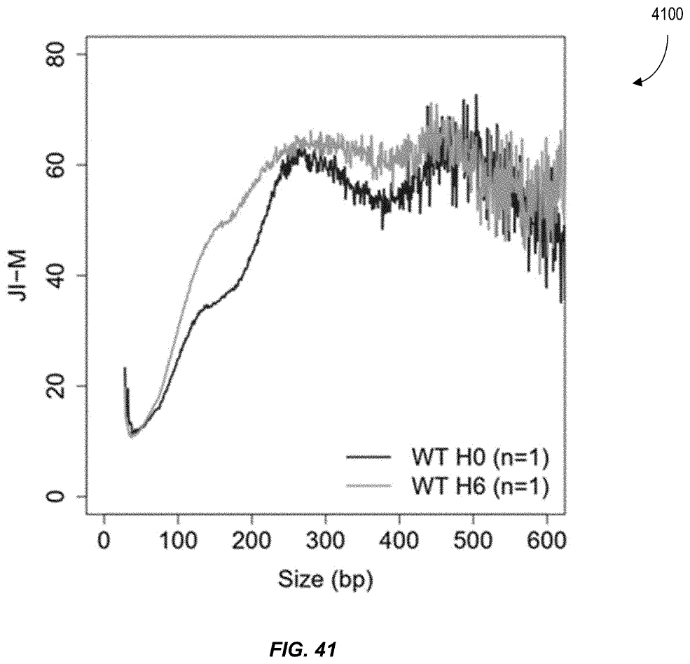

FIG. 41 shows a graph 4100 that identifies JI-M values across different fragment sizes between 0-hour heparin incubation and 6-hour heparin incubation from wildtype mice. As shown in the graph 4100, the existence of DNASE1 in WT mice (JI-M: 34.01) leads to a 62.57% increase in jaggedness after 6-hour heparin incubation (JI-M: 46.72). Thus, the overall JI-M distribution of WT mice DNA molecules with different heparin incubation time shows that DNA molecules from 6-hour heparin incubation plasma bear higher jaggedness.

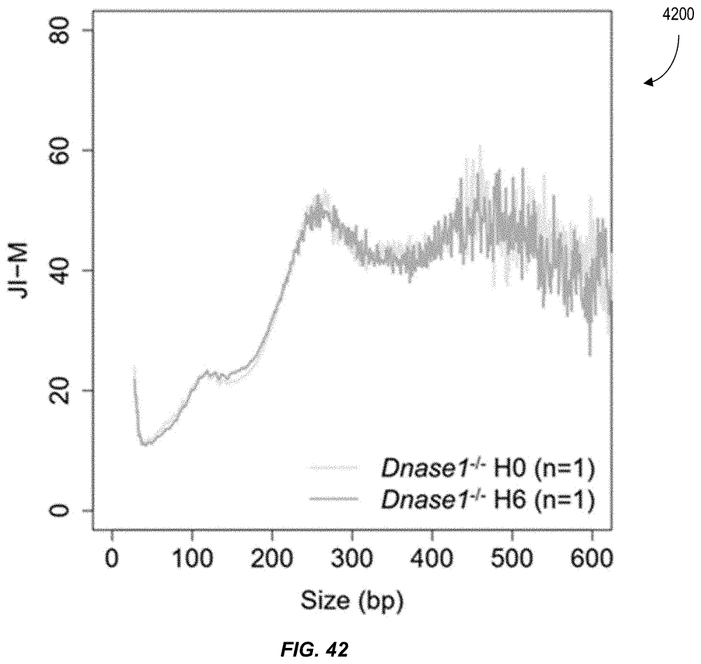

FIG. 42 shows a graph 4200 that identifies JI-M values across different fragment sizes between 0-hour incubation and 6-hour incubation with heparin for DNASE1″ mice. The graph 4200 shows that, when DNASE1 is knocked out, the increase of jaggedness in 6-hour heparin incubation disappears. The JI-M distribution across fragment size thus in DNASE1″ cfDNA molecules shows an overall similar trend between 0-hour and 6-hour incubation. Compared with the significant increase of jaggedness in wildtype mice after 6-hour-heparin incubation, the overall trend of jaggedness across sizes in DNASE1^{-/-} mice were found to be nearly overlapped.

These data suggested that with heparin-based enhancement of the activity of DNASE1, jaggedness increased especially in short plasma DNA fragments, which means that DNASE1 might be responsible for jagged end generation regarding short plasma DNA fragments.

4. Methods for Determining Genetic Disorders

Various techniques can be used to detect genetic disorders, e.g., associated with a nuclease. The genetic disorders can relate to a mutation (e.g., a deletion) of a nuclease corresponding to a particular gene. Such a mutation can cause the nuclease to not exist or to function in an irregular manner. Accordingly, an extent of changes in expression levels of the affected nuclease can be determined. In some instances, jaggedness index values corresponding to a plurality of nuclei acid molecules in the biological sample can be determined to identify the changes in nuclease expression levels. These jaggedness index values can be used as reference values, which can be compared with a jaggedness index value determined for a subject to determine genetic disorders. Examples of such methods are described in the following flowcharts. Techniques described for one flowchart are applicable to other flowcharts, and are not repeated for the sake of being concise.

a) Detecting Genetic Disorder Using Incubation Over Time

Different amounts of incubation of a sample can result in different jaggedness index values (e.g., FIGS. 40 and 41) depending on whether the genetic disorder exists. As a particular jaggedness index value can depend on whether a particular nuclease expressed and functioning properly, a change in such behavior from normal can indicate the genetic disorder exists.

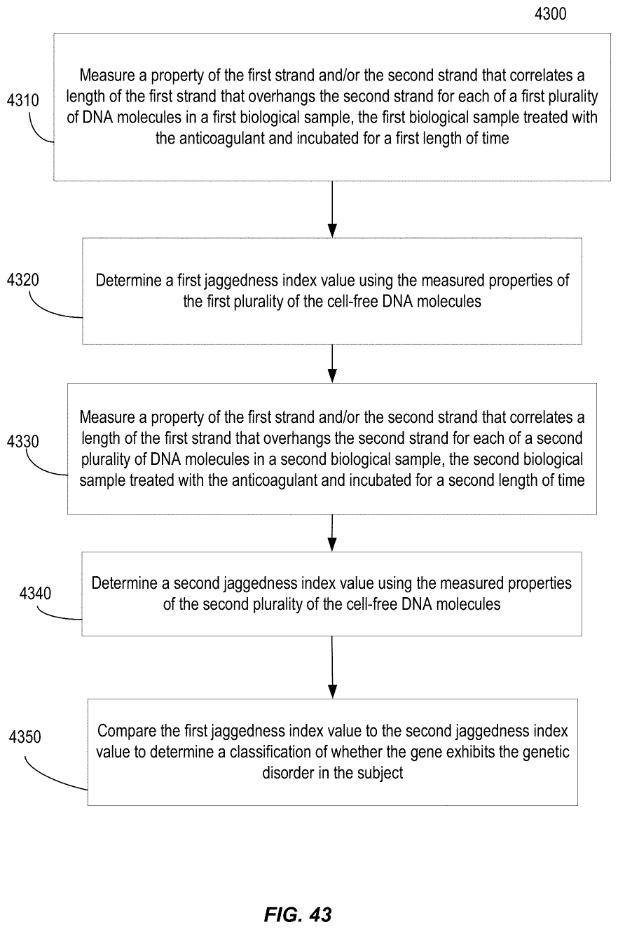

FIG. 43 shows a flowchart illustrating a method 4300 for detecting a genetic disorder for a gene associated with a nuclease using biological samples including cell-free DNA according to embodiments of the present disclosure. Method 4300 and others method herein can be performed entirely or partially with a computer system, including being controlled by a computer system. As examples, a gene can be associated with a nuclease by coding for the nuclease, having epigenetic markers for its transcription, having its RNA transcripts present, having variably spliced RNA, or having its RNA variably translated. The genetic disorder may be in only certain tissue (e.g., tumor tissue). Accordingly, the detection of the genetic disorder may be used to determine a level of cancer.

At block 4310, a property of the first strand and/or the second strand that correlates a length of the first strand that overhangs the second strand is measured for each cell-free DNA molecule of a first plurality of the cell-free DNA molecules of a first biological sample. The first biological sample can treated with an anticoagulant and incubated for a first length of time. The incubation can be at a certain temperature or higher, e.g., above 5°, 10°, 15°, 20°, 25°, or 30° Celsius. Storage at lower temperatures may not count as part of the incubation time. The first length of time can be zero. In other implementations, the first biological sample is incubated for the first length of time without being treated with an anticoagulant. As examples, the anticoagulant can be EDTA or heparin. The EDTA can help to inhibit plasma nucleases (e.g., DNASE1 and DNASE1L3) to preserve cfDNA for analysis.

In some instances, a measured property includes a higher methylation level of the first strand, in which the higher methylation level is correlated with a longer length of the first strand that overhangs the second strand. In another example, a measured property includes a lower methylation level of the first strand, in which the lower methylation level is correlated with a longer length of the first strand that overhangs the second strand. In some instances, the property is a methylation status at one or more sites at end portions of the first strands and/or second strands of each of the plurality of nucleic acid molecules. In other instances, the property is a length of the first strand and/or the second strand that is proportional to the length of the first strand that overhangs the second strand.

In several embodiments, the plurality of the cell-free DNA molecules (for which the property is measured) is configured to have a size within a specified range, e.g., 130 to 160 bps. Other size ranges, including but not limited to, 100-130 bp, 110-140 bp, 120-150 bp, 140-170 bp, 150-180 bp, 160-190 bp, 170-200 bp, 180-210 bp, 190-220 bp, and other size ranges or multiple combinations of different size ranges, would be used in other embodiments.

In some embodiments, jagged ends across different size ranges and different genomic locations can be used as training data for machine learning algorithms to determine fractional concentration of clinically-relevant DNA, differentiate abnormal cells from normal tissue, and the link. The machine learning algorithms may include, but not limited to, linear regression, logistic regression, deep recurrent neural network, Bayes classifier, hidden Markov model (HMM), linear discriminant analysis (LDA), k-means clustering, density-based spatial clustering of applications with noise (DBSCAN), random forest algorithm, and support vector machine (SVM).

At block 4320, a first jaggedness index value is determined using the measured properties of the first plurality of the cell-free DNA molecules. In some embodiments, the first jaggedness index value provides a collective measure that a strand overhangs another strand in the first plurality of the cell-free DNA molecules. In some instances, the first jaggedness index value identifies a methylation level over the plurality of nucleic acid molecules at one or more sites of end portions of the first strands and/or second strands. In some embodiments, the first jaggedness index value corresponds to the measured properties of the first plurality of the cell-free DNA molecules having size within a specified range, e.g., 130 to 160 bps.

At block 4330, a property of the first strand and/or the second strand that correlates a length of the first strand that overhangs the second strand is measured for each cell-free DNA molecule of a second plurality of the cell-free DNA molecules of a second biological sample. The second biological sample can be treated with the anticoagulant and incubated for a second length of time that is greater than the first length of time. In other implementations, the second biological sample can be incubated without being treated by the anticoagulant. The length of time can include a temperature factor, e.g., a higher temperature can act as a weighting factor multiplied by a time unit to obtain the length of time. In this manner, a greater/same amount of cell death can occur in a sample/shorter amount of time due to the incubation at a higher temperature. Step 4330 may be performed in a similar manner as step 4310.

At block 4340, a second jaggedness index value is determined using the measured properties of the second plurality of the cell-free DNA molecules. In some embodiments, the second jaggedness index value provides a collective measure that a strand overhangs another strand in the second plurality of the cell-free DNA molecules. In some instances, the second jaggedness index value identifies a methylation level over the plurality of nucleic acid molecules at one or more sites of end portions of the first strands and/or second strands. In some embodiments, the second jaggedness index value corresponds to the measured properties of the second plurality of the cell-free DNA molecules having size within a specified range, e.g., 130 to 160 bps. Step 4340 may be performed in a similar manner as step 4320.

At block 4350, the first jaggedness index value is compared to the second jaggedness index value to determine a classification of whether the gene exhibits the genetic disorder in the subject. In some implementations, comparing the first jaggedness index value to the second jaggedness index value includes determining whether the first jaggedness index value differs from the second jaggedness index value by at least a threshold amount, and can include which jaggedness index value is larger than the other when there is a statistically significant difference or other separation value. Accordingly, the classification can be that the genetic disorder exists when the first jaggedness index value is within a threshold of the second jaggedness index value.

In some instances, the genetic disorder includes rheumatoid arthritis, type 1 diabetes, multiple sclerosis, systemic lupus erythematosus (SLE), inflammatory bowel disease, psoriasis, scleroderma, autoimmune thyroiditis, or any combinations thereof. The classification can be a level or severity of the disorder, e.g., from whether a coding gene for the nuclease is missing in both chromosomes, in only one chromosome, are missing in only certain tissue, or the mutation reduces expression but does not eliminate the existence of the nuclease. Such a partial reduction in the expression of the nuclease can occur when the mutation (e.g., a deletion) is only in certain tissue or when the mutation is within a supporting region, e.g., in a non-coding region such as miRNA that affects the level of expression of the nuclease. The different levels or severity of the genetic disorder, as a result of differing amounts of difference relative to the reference level. Multiple reference levels can be used to determine the difference classifications.

In some examples, when the first jaggedness index value is within a threshold of the jaggedness index value amount, the classification can be that the genetic disorder exists. In some embodiments, the comparison can include determining a separation value between the first jaggedness index value and the second jaggedness index value. The separation value can be compared to a reference value (e.g., a cutoff) to determine the classification. The reference value can be a calibration value determined using calibration (reference) samples, which have known classifications and can be analyzed collectively to determine a reference value or calibration function (e.g., when the classifications are continuous variables). The first jaggedness index value and second jaggedness index value are examples of a parameter value that can be compared to a reference/calibration value. Such techniques can be used for all methods herein.

The one or more calibration values can be one or more reference values or be used to determine a reference value. The reference values can correspond to particular numerical values for the classifications. For example, calibration data points (calibration value and measured property, such as nuclease activity or level of efficacy) can be analyzed via interpolation or regression to determine a calibration function (e.g., a linear function). Then, a point of the calibration function can be used to determine the numerical classification as an input based on the input of the measured amount or other parameter (e.g., a separation value between two amounts or between a measured amount and a reference value). Such techniques may be applied to any of the method described herein.

The type of genetic disorder being tested can provide the type of criteria used for determining whether the disorder exists, as the cfDNA behavior will be different.

As an example, the genetic disorder can include a deletion of the gene. As examples, the genes can be DFFB, DNASE1L3, or DNASE1. The nuclease can be one that cuts intracellular DNA, e.g., DFFB or DNASE1L3. The nuclease can be one that cuts extracellular DNA, e.g., DNASE1 or DNASE1L3.

b) Detecting Genetic Disorder Using Reference Value

As described above, a difference or other separation value (e.g., whether small or large) in jaggedness between samples with different incubations can be used to classify a genetic disorder for a gene associated with a nuclease. Alternatively, a jaggedness index value determined from a measured property of nucleic acid molecules can be compared to a reference value. Such a reference value can correspond to a jaggedness index value measured in a healthy subject.

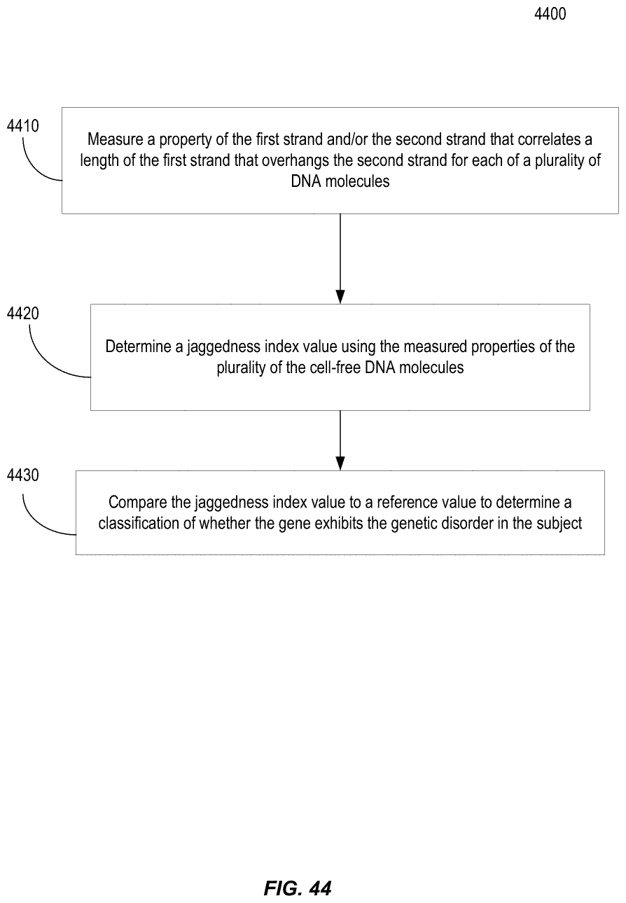

FIG. 44 shows a flowchart illustrating a method 4300 for detecting a genetic disorder for a gene associated with a nuclease using a biological sample including cell-free DNA according to embodiments of the present disclosure. Similar techniques as used for method 4300 may be used in method 4400. As examples, the gene is DNASE1L3, DFFB, or DNASE1. In some instances, the genetic disorder includes rheumatoid arthritis, type 1 diabetes, multiple sclerosis, systemic lupus erythematosus (SLE), inflammatory bowel disease, psoriasis, scleroderma, autoimmune thyroiditis, or any combinations thereof.

At block 4410, a property of the first strand and/or the second strand that correlates a length of the first strand that overhangs the second strand is measured for each cell-free DNA molecule of a plurality of the cell-free DNA molecules of a biological sample. In some instances, a measured property includes a higher methylation level of the first strand, in which the higher methylation level is correlated with a longer length of the first strand that overhangs the second strand. In another example, a measured property includes a lower methylation level of the first strand, in which the lower methylation level is correlated with a longer length of the first strand that overhangs the second strand. In some instances, the property is a methylation status at one or more sites at end portions of the first strands and/or second strands of each of the plurality of nucleic acid molecules. In other instances, the property is a length of the first strand and/or the second strand that is proportional to the length of the first strand that overhangs the second strand. Similar techniques as used for block 4310 of FIG. 43 may be used in block 4410.

In some instances, the biological sample can treated with an anticoagulant and incubated for a specified amount of time. The incubation can be at a certain temperature or higher, e.g., above 5°, 10°, 15°, 20°, 25°, or 30° Celsius. Storage at lower temperatures may not count as part of the incubation time. The first length of time can be zero. In other implementations, the biological sample is incubated for the specified amount of time without being treated with an anticoagulant. As examples, the anticoagulant can be EDTA or heparin. The EDTA can help to inhibit plasma nucleases (e.g., DNASE1 and DNASE1L3) to preserve cfDNA for analysis.

At block 4420, a jaggedness index value is determined using the measured properties of the plurality of the cell-free DNA molecules. In some embodiments, the jaggedness index value provides a collective measure that a strand overhangs another strand in the first plurality of the cell-free DNA molecules. In some instances, the jaggedness index value identifies a methylation level over the plurality of nucleic acid molecules at one or more sites of end portions of the first strands and/or second strands. In some embodiments, the jaggedness index value corresponds to the measured properties of the plurality of the cell-free DNA molecules having size within a specified range, e.g., 130 to 160 bps. For example, a jaggedness index value for detecting SLE in a biological sample can correspond to the measured properties of the plurality of cell-free DNA molecules having a size within 200-300 bps. Similar techniques as used for block 4320 of FIG. 43 may be used in block 4420.

At block 4430, the jaggedness index value is compared to a reference value to determine a classification of whether the gene exhibits the genetic disorder in the subject. In various embodiments, comparing the first amount to the second amount can include: (1) determining whether the jaggedness index value differs from the reference value by at least a threshold amount or the difference is less than the threshold amount; (2) determining whether the jaggedness index value is less than the reference value by at least a threshold amount; or (3) determining whether the jaggedness index value is greater than the reference value by at least a threshold amount. The jaggedness index value is an example of a parameter value and the reference value can be a calibration value or determined from calibration values of calibration samples. In some instances, the classification additionally identifies whether the gene exhibits a symptomatic or asymptomatic disorder (e.g., active SLE) in the subject.

The reference value can be a calibration value determined using calibration (reference) samples, which have known classifications and can be analyzed collectively to determine a reference value or calibration function (e.g., when the classifications are continuous variables). For example, the nuclease activity can be a continuous variable, and the comparison of the amount to the reference value can be determine by inputting the amount to a calibration function, e.g., as is described herein. With respect to known classifications, the reference value can be determined from one or more reference samples that do not have the genetic disorder. Additionally or alternatively, the reference value is determined from one or more reference samples that have the genetic disorder. Similar techniques as used for block 4350 may be used in block 4430.

E. Jagged-End Analysis for Monitoring Nuclease Activity

Jaggedness of cell-free DNA can be determined to monitor the activity of a nuclease, e.g., DFFB, DNASE1, and DNASE1L3. Such activity can be from internal nucleases (i.e., as a natural process of the body) and/or from the result of adding a nuclease, e.g., DNASE1. Such monitoring can be used to determine a change in a genetic disorder for the efficacy of a treatment. For example, DNASE1 can be used to treat a subject. An effect of the treatment can be measured by analyzing the T-end fragment percentage or size. In some embodiments, DNASE1 (e.g., exogenously added) can be used to treat auto-immune conditions, such as SLE. Depending on the determination of the activity, the dosage of treatment of the nuclease can be changed. In some instances, activity of an exonuclease (e.g., exonuclease T) is monitored.

The determination of abnormal nuclease activity (e.g., above or below a reference value corresponding to normal/healthy values) can indicate a level of pathology alone or in combination with other factors. The pathology can be cancer.

1. Jaggedness in Determining Cutting Properties of Nucleases

Apart from the study in mouse models, jaggedness can also be used for revealing the cutting properties of commercial-available enzymes, such as exonucleases and endonucleases, and Cas9. For instance, exonuclease T (ExoT) is a common-use enzyme to generate blunt ends. We studied the jagged end detection with and without ExoT treatment on the basis of DNA molecule carrying a known jagged end (e.g., synthetic oligonucleotides).

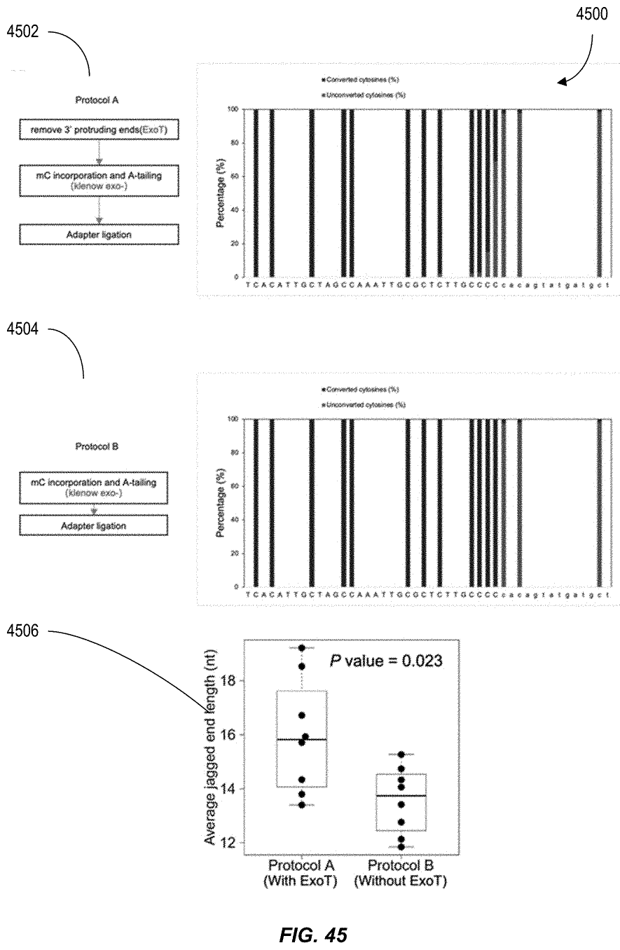

FIG. 45 shows protocols 4500 identifying jaggedness of annealed dsDNA treated with or without ExoT. Protocol 4502 illustrates a process for preparing a library with ExoT, which shows that a few extra sites upstream to the jagged end site would be incorporated with mC in annealed oligo control. The letters in upper case represent the double-stranded region. The letters in lower case represent the single-stranded jagged end. As shown in the protocol 4502, 68.8% of 1 bp upstream of the jagged end site displayed the incorporation of methylated cytosines, 15.04% of 2 bp upstream of the jagged end site displayed the incorporation of methylated cytosines and 2.71% of 3 bp upstream of the jagged end site displayed the incorporation of methylated cytosines.

Protocol 4504 illustrates a process for preparing a library prepared without ExoT, which no such extra incorporation of mC in the upstream of the jagged end site in annealed oligo control. In contrast to the protocol 4502, an extra incorporation of methylated cytosines nearby the jagged end was not observable in samples without ExoT treatment. Box plot 4506 shows averaged jagged end length in 8 paired samples with two different library preparation process. Compared with DNA libraries prepared without ExoT (median JI-M value: 13.74; range 11.84-15.27), a median of 15.16% of increase of jaggedness in human samples was found (median JI-M value 15.82; range 13.40-19.21) (FIG. 10C). These results suggested that ExoT would bear the 3′ to 5′ exonuclease activity even in double strand region.

2. Methods for Monitoring Nuclease Activity

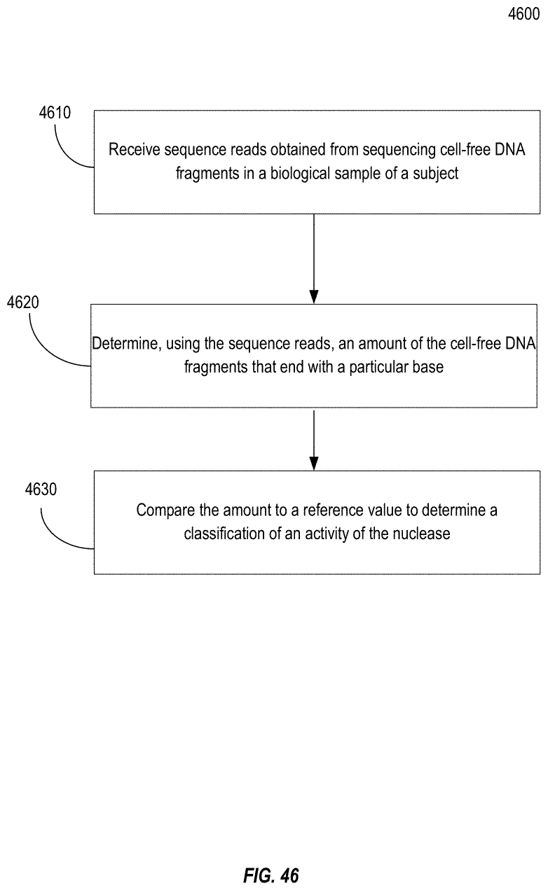

FIG. 46 is a flowchart illustrating a method 4600 for monitoring activity of a nuclease using a biological sample including cell-free DNA according to embodiments of the present disclosure. In some embodiments, the nuclease is an endonuclease, such as DNASE1, DFFB, DNASE1L3, ENDOG, APEX1, FEN1, DNASE1L1, DNASE1L2, or DNASE2. Additionally or alternatively, the nuclease is an exonuclease, such as ExoT, EXOG, TREX1, or EXO1. Aspects of method 4600 can be performed in a similar manner as other methods described herein.

At block 4610, a property of the first strand and/or the second strand that correlates a length of the first strand that overhangs the second strand is measured for each cell-free DNA molecule of a plurality of the cell-free DNA molecules of a biological sample. In some instances, a measured property includes a higher methylation level of the first strand, in which the higher methylation level is correlated with a longer length of the first strand that overhangs the second strand. In another example, a measured property includes a lower methylation level of the first strand, in which the lower methylation level is correlated with a longer length of the first strand that overhangs the second strand. In some instances, the property is a methylation status at one or more sites at end portions of the first strands and/or second strands of each of the plurality of nucleic acid molecules. In other instances, the property is a length of the first strand and/or the second strand that is proportional to the length of the first strand that overhangs the second strand. Similar techniques as used for block 4310 of FIG. 43 may be used in block 4610.

At block 4620, a jaggedness index value is determined using the measured properties of the plurality of the cell-free DNA molecules. In some embodiments, the jaggedness index value provides a collective measure that a strand overhangs another strand in the first plurality of the cell-free DNA molecules. In some instances, the jaggedness index value identifies a methylation level over the plurality of nucleic acid molecules at one or more sites of end portions of the first strands and/or second strands. In some embodiments, the jaggedness index value corresponds to the measured properties of the first plurality of the cell-free DNA molecules having size within a specified range, e.g., 130 to 160 bps. Similar techniques as used for block 430 of FIG. 43 may be used in block 4620.

At block 4630, the jaggedness index value is compared to a reference value to determine a classification of an activity of the nuclease. In some embodiments, if the activity is below the reference value, the subject can be classified as having a disorder. In such a case, the subject can be treated, e.g., as described herein. The classification can be a numerical classification value, which can be compared to a cutoff to determine a second classification of whether a gene associated with the nuclease exhibits a genetic disorder in the subject.

The reference value can be a calibration value determined using calibration (reference) samples, which have known classifications and can be analyzed collectively to determine a reference value or calibration function (e.g., when the classifications are continuous variables). For example, the nuclease activity can be a continuous variable, and the comparison of the amount to the reference value can be determine by inputting the amount to a calibration function, e.g., as is described herein.

In some instances, the reference value is determined using one or more reference samples having a known or measured classification for the activity of the nuclease. The activity of the nuclease for the one or more reference samples can be measured as described herein, e.g., fluorometric or spectrophotometric measurement of cfDNA quantity, which may be done on its own or before, after, and/or in real-time with, the addition of a nuclease-containing sample. Another example is using radial enzyme diffusion methods. The calibration values can be measured in the one or more reference samples, thereby providing calibration data points comprising the two measurements for the reference/calibration samples. The one or more reference samples can be a plurality of reference samples. A calibration function can be determined that approximates calibration data points corresponding to the measured activities and measured amounts for the plurality of reference samples, e.g., by interpolation or regression.

### VI. Combined Analysis of Jagged Ends and End Signatures

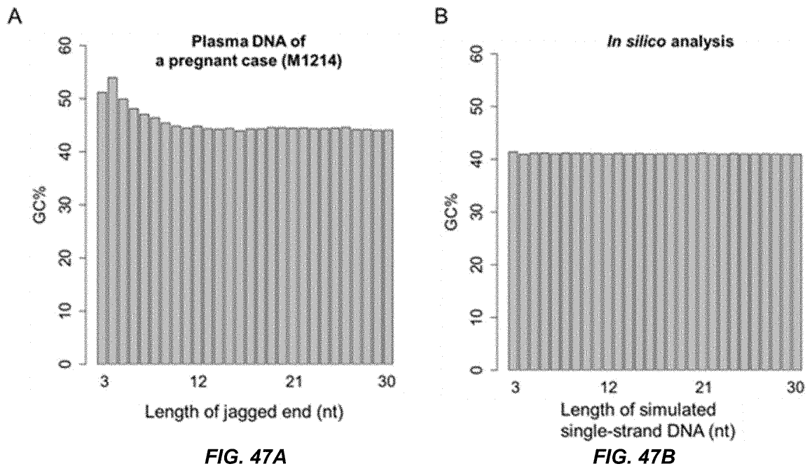

Both end signatures and jagged ends can be used together to represent nuclease expression levels. For example, FIGS. 47A and 47B show example graphs depicting the relationship between GC % and jagged end length according to some embodiments. We found that single-stranded DNA with short jagged ends (e.g., at 3, 4, and 5 nt) contained higher GC % (mean: 51%) than those with long jagged ends (e.g., >12 nt; mean GC %: 45%) (FIG. 47A). However, such patterns were absent in the result which was randomly generated in silico from the human reference genome (FIG. 47B). These results suggested that the base compositions were not even across different jagged end lengths. Embodiments can use this synergy between sequence motifs and a jaggedness index. In one embodiment, we found that the motif diversity score would give the largest AUC value (AUC: 0.84) for those molecules at a jagged end length of 6, which was higher than that using molecules without selection according to jagged end lengths (AUC: 0.77). Thus, these results suggested that one could improve the differentiating power by selectively analyzing those molecules with a certain jagged end length or desired ranges.

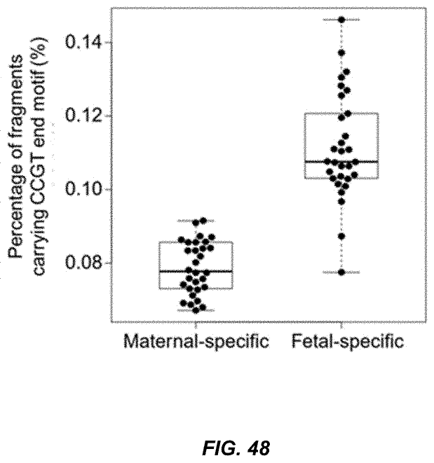

FIG. 48 shows a boxplot of the percentage of fragments carrying CCGT end motif according to some embodiments. The abundance of end motif CCGT was higher in the fetal DNA molecules (median: 0.079; range: 0.067-0.09) than that in maternal DNA molecules (median: 0.11; range: 0.078-0.15) (P value <0.0001) (FIG. 34).

A. Fractional Concentration of Clinically-Relevant DNA

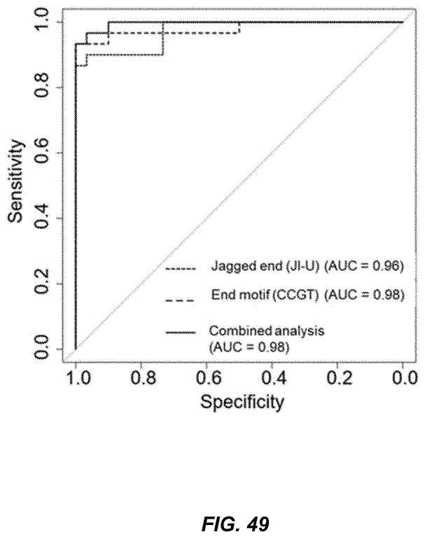

The combined analysis of end signatures and jagged ends can be used to determine a characteristic of a tissue type, in which the characteristic corresponds to a fractional concentration of clinically-relevant DNA. FIG. 49 shows a classification power analysis for differentiating the maternal and fetal DNA fragments using jagged end index (JI-U), end motif (CCGT), and combined end motif and jagged end analysis according to some embodiments. As an example, the combined analysis aforementioned was carried out as below:
(1) a dataset including patients with and without HCC was classified into two classes (i.e. positive cases and negative cases) based on the abundance of end motif CCGT which was compared to a certain cutoff.
(2) Then, the positive cases determined in the above step was further classified into two classes (i.e. positive cases and negative cases) based on the jagged end index which was compared to a certain cutoff.
(3) A case which was persistently classified as positive in two steps of binary classification was deemed positive. The cutoffs used in above processes of binary classification could be varied, forming a number of resultant classification models. Among those classification models, one could determine an optimal model using a combined analysis with end motifs and jagged ends. In one embodiment, this combined analysis would be expanded to include two or more end motifs and other fragmentomic features such as, but not limited to, fragment size, fragment size-fractionated jagged ends, preferred ends, and nucleosome footprints of plasma DNA molecules. In yet other embodiments, one or more of these metrics could be combined with other non-fragmentomic features of plasma DNA, e.g., methylation status.

As shown in FIG. 49, the combined end motif and jagged end analysis showed a higher AUC (0.98), as compared to the AUC values of the individual analyses (Jagged ends=0.96 AUC; End motif=0.96 AUC). Thus, the combined analysis can be used to improve accuracy for differentiating abnormal tissues from normal tissues, determining fractional concentration of clinically-relevant DNA, differentiating tissue types, and the like.

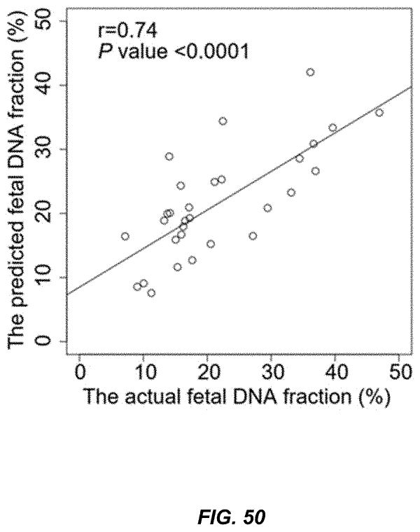

FIG. 50 shows a scatter plot between the predicted fetal DNA fractions and actual fetal DNA fractions in plasma DNA samples of pregnant women, according to some embodiments. The actual fetal DNA fractions were deduced by SNP approach (Lo et al. Sci Transl Med. 2010; 2:61ra91). Referring to FIG. 50, one could use a regression analysis using end motifs and jagged ends to predict the fetal DNA fraction in the plasma DNA of a pregnant woman. For illustration purpose, we could use a leave-one-out analysis in which one sample was deemed as a testing sample and the remaining samples were used to train a mathematical model (e.g., a multiple linear regression model) and to repeat this process till all samples has been tested. As an example, the end motif CCGT and jagged end index metrics as independent variables were used for fitting a multiple linear regression model with regard to the fetal DNA fraction as a dependent variable. In the training process, the actual fetal DNA fractions could, in one embodiment, be determined by SNP approach (e.g., according to Lo et al. Sci Transl Med. 2010; 2:61ra91). In one embodiment, the predicted fetal DNA fraction was correlated with the actual fetal DNA fractions (r=0.74 and P value <0.0001) (FIG. 50). Such combined end motif and jagged end analysis for deducing the fetal DNA fraction was superior to the model using a single metric CCGT end motif (r=0.72) or jagged end index (0.3).

The combined analysis of end signatures and jagged ends can also be used to determine a characteristic of a tissue type in a biological sample, in which the characteristic corresponds to a fraction of abnormal cells (e.g., tumor DNA).

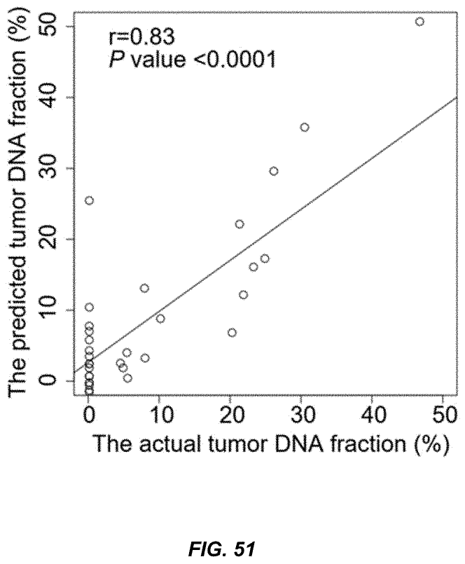

FIG. 51 is a scatter plot between the predicted tumor DNA fractions and actual tumor DNA fraction in patients with HCC, according to some embodiments. The actual tumor DNA fractions was determined by copy number aberrations (Adalsteinsson et al. Nat Commun. 2017; 8:1324). In another embodiment, in patients with HCC, we used the abundance of end motif ACGA and jagged end index (JI-U) to fit a multiple linear regression with regard to the tumor DNA fraction. In the training process, the actual tumor DNA fractions were determined by copy number aberrations (Adalsteinsson et al. Nat Commun. 2017; 8:1324). As shown in FIG. 50, based on leave-one-out analysis, the correlation coefficient between the predicted and actual tumor DNA fraction was 0.83 (P value <0.0001). This result suggested that the combined end motif and jagged end analysis allowed for deducing the tumor DNA fractions in patients with HCC.

In some instances, different statistical approaches are used to selectively combine end motifs and jagged ends, for example but not limited to, including logistic regression, support vector machines (SVM), decision tree, CART algorithm (Classification and Regression Trees), naïve Bayes classification, clustering algorithm, principal component analysis, singular value decomposition (SVD), t-distributed stochastic neighbor embedding (tSNE), artificial neural network, ensemble methods which construct a set of classifiers and then classify new data points by taking a weighted vote of their prediction, etc.

B. Methods for Determining Characteristic Value of Target Tissue Using the Combined Analysis

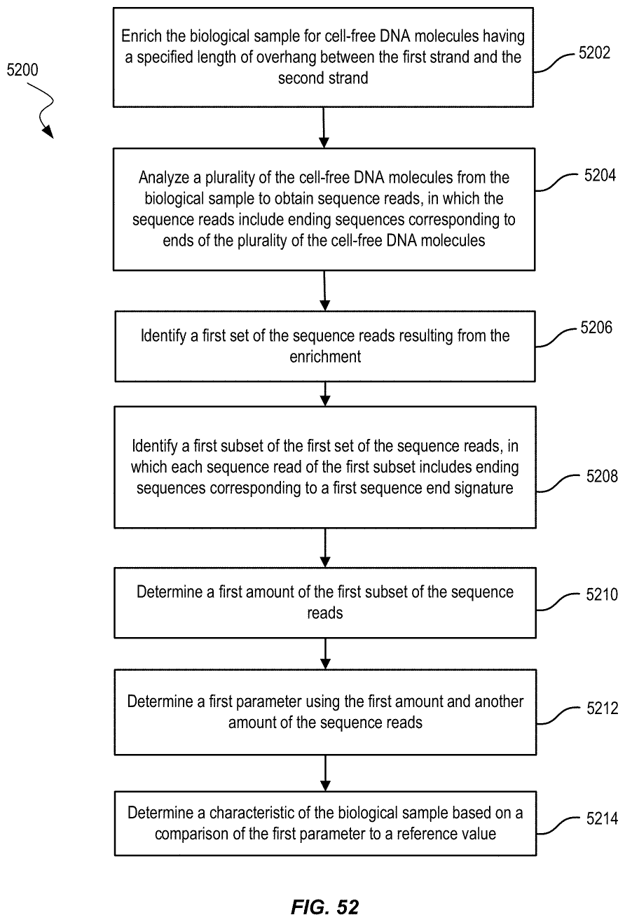

FIG. 52 is a flowchart illustrating a method of determining a characteristic of a biological sample based on end signatures derived from cell-free DNA molecules having jagged ends, according to some embodiments. In some embodiments, the biological sample includes cell-free DNA molecules, in which each of the cell-free DNA molecules is partially or completely double-stranded with a first strand having a first portion and a second strand. In some instances, the first portion of the first strand of at least some of the cell-free DNA molecules has no complementary portion from the second strand, is not hybridized to the second strand, and is at a first end of the first strand. In some embodiments, the characteristic of a target tissue type indicates a gestational age in placental tissues, or conditions relating to the placental tissue including preeclampsia, preterm birth, fetal chromosomal aneuploidies, metabolic disorders and/or fetal genetic disorder. The characteristic of the target tissue type may also be used to differentiate tissue types, such as differentiating liver-derived DNA molecules and DNA molecules mainly of hematopoietic origin.

At step 5202, the biological sample is enriched for cell-free DNA molecules having a specified length of overhang between the first strand and the second strand. Different techniques may be used to enrich cell-free DNA molecules having the specified length of overhang between the first strand and the second strand, including jagged end specific hybridization based targeted capture, jagged end specific adaptor ligation based amplicon sequencing, and digital PCR (e.g., droplet digital PCR).

At step 5204, a plurality of the cell-free DNA molecules from the biological sample are analyzed to obtain sequence reads. In some embodiments, the sequence reads include ending sequences corresponding to ends of the plurality of the cell-free DNA molecules. As described herein, sequence read may be obtained in a variety of ways, e.g., using sequencing techniques (e.g., using a sequencing-by-synthesis approach (e.g., Illumina), or single molecule sequencing (e.g., by the single molecule, real-time system from Pacific Biosciences, or by nanopore sequencing (e.g., by Oxford Nanopore Technologies), or using probes, e.g., in hybridization arrays or capture probes. In some embodiments, the sequencing process may be preceded by amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

At step 5206, a first set of the sequence reads resulting from the enrichment are identified. In some embodiments, paired-end sequencing is used to obtain sequence reads, which two sequence reads are obtained from the two ends of a DNA fragment, e.g., 30-120 bases per sequence read.

At step 5208, a first subset of the first set of the sequence reads is identified. In some embodiments, each sequence read of the first subset includes ending sequences corresponding to a first sequence end signature. In some embodiments, the first set of sequence reads include ending sequences corresponding to ends of the plurality of cell-free DNA molecules. The ending sequences having the first sequence end signature may be determined using a reference genome, e.g., to identify bases just before a start position or just after an end position. Such bases will still correspond to ends of cell-free DNA fragments, e.g., as they are identified based on the ending sequences of the fragments. Step 5208 may be performed in a similar manner as step 2608 of FIG. 26.

At step 5210, a first amount of the first subset of the sequence reads is determined. In some embodiments, the first amount of the first set of the sequence reads may be counted (e.g., stored in an array in memory). Step 5210 may be performed in a similar manner as step 2610 of FIG. 26.

At step 5212, a first parameter is determined using the first amount and potentially another amount of the sequence reads. In some examples, both of such amounts can be separate parameters. The other amount can take various forms, e.g., corresponding to a total number of sequence reads and/or DNA molecules analyzed. As another example, the other amount can correspond to an amount of one or more other sequence end signatures (end motifs). The first parameter can be a ratio of amounts between two plasma end motifs (e.g., CCCA/AAAT). Step S212 may be performed in a similar manner as step 2612 of FIG. 26.

At step 5214, a characteristic of the biological sample is determined based on a comparison of the first parameter to a reference value. For example, the determined characteristic can include a gestational age or range (e.g., 8 weeks, 9-12 weeks), e.g., when a nuclease is differentially regulated between fetal tissue and maternal tissue. In another example, the determined characteristic can be a particular tissue type (e.g., liver cells) relative to the other tissue type (e.g., hematopoietic cells). The characteristic of the target tissue type may also indicate a particular condition of the target tissue type (e.g., HCC, preeclampsia, preterm birth). In another example, the determined characteristic can be a size or nutrition status of an organ corresponding a particular tissue type (e.g., liver cells). In yet another example, the determined characteristic can include a fraction of clinically-relevant DNA in a biological sample. In some embodiments, clinically-relevant DNA include fetal DNA, tumor-derived DNA, or transplant DNA. Step 5214 may be performed in a similar manner as step 2612 of FIG. 26.

### VII. Example Techniques for Detecting Jagged Ends in DNA Molecules

Various example techniques for detecting jagged ends in DNA molecules are described below, which may be implemented in various embodiments.

A. Enriching Jagged Ends Based on Jagged-End Specific Hybridization

In another embodiment, one would physically enrich those molecules with certain jagged ends which showed the greatest discriminative power. Such physical enrichment could include, but not limited to, jagged end specific hybridization based targeted capture, jagged end specific ligation based PCR amplification, and jagged end specific ligation based capture. In another embodiment, real-time PCR (also called quantitative PCR or qPCR) and droplet digital PCR (ddPCR) would be used for detecting and quantify jagged ends.

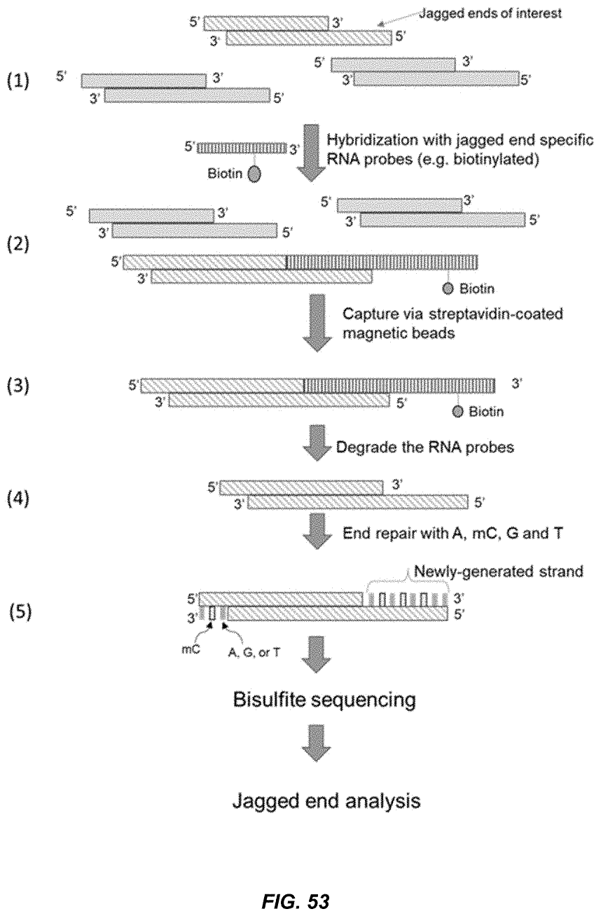

FIG. 53 illustrates an example of a method using jagged end specific hybridization based targeted capture for enriching a certain number of ends of interest, in accordance with some embodiments. In one embodiment for physical enrichment analysis, one could use jagged end specific hybridization based targeted capture for enriching the jagged ends of interest. Biotinylated RNA probes which could be specifically hybridized to the jagged ends of interest were designed (illustrated in steps 1 and 2). The jagged ends of interest which would be hybridized with biotinylated probes could be pulled down by the streptavidin-coated magnetic beads (illustrated in step 3). The RNA probes would be degraded by ribonucleases such as RNase H (illustrated in step 4). The jagged ends of interest would be enriched in the pull-down material and subjected to DNA end repair with adenines (A), guanines (G), thymines (T), and methylated C (5 mC) (illustrated in step 5). Hence, the single-stranded strand attached to the molecules carrying the jagged ends of interest would be filled in with 5 mC and become blunt molecules for bisulfite sequencing. The information concerning jagged ends of interest could be determined from the results of bisulfite sequencing according to, but not limited to, the approaches described in US Patent Publication No. 2020/0056245 A1, filed Jul. 23, 2019, the entire contents of which are incorporated herein by reference in its entirety and for all purposes. In one embodiment, one or more different jagged ends were analyzed together, e.g., ratios or deviations between readouts of different jagged ends for practical applications.

B. Enriching Jagged Ends Based on Jagged-End Specific Adapter Ligation

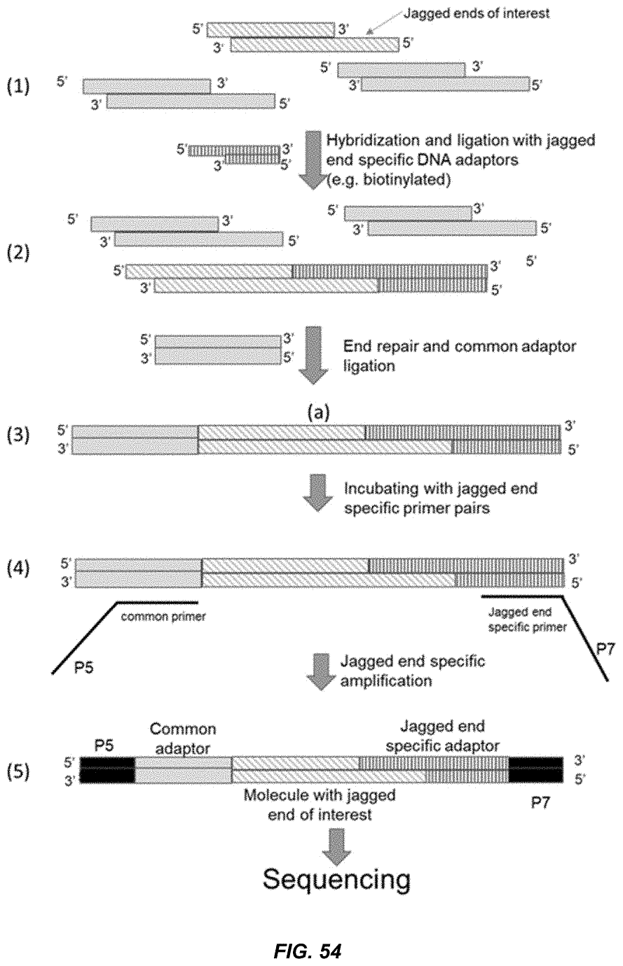

FIG. 54 illustrates an example of a method using jagged end specific adaptor ligation based amplicon sequencing for enriching a certain number of ends of interest, in accordance with some embodiments. In one embodiment for physical enrichment analysis, the jagged ends of interest for a molecule would be specifically ligated with an adaptor (i.e. jagged end specific adaptor (illustrated in step 1 and 2). The other end of the same molecule would become blunt after DNA end repair, which could be ligated with a universal adaptor (i.e. common adaptor) (illustrated in step 3). A molecule ligated with both common adaptor and jagged end specific adaptor were subjected to PCR amplification using a common primer with e.g., Illumina P5 sequence and jagged end specific primer with e.g., Illumina P7 sequence (illustrated in step 4 and 5). The amplified product could be used for determining the jagged ends of interest. In one embodiment, both termini of a DNA molecule could be ligated with specific adaptors, thus allowing for detecting jagged ends of interest present in two ends of a molecule. In one embodiment, one or more different jagged ends were analyzed together, e.g., ratios or deviations between readouts of different jagged ends for practical applications.

C. Detection of Jagged Ends of Interest

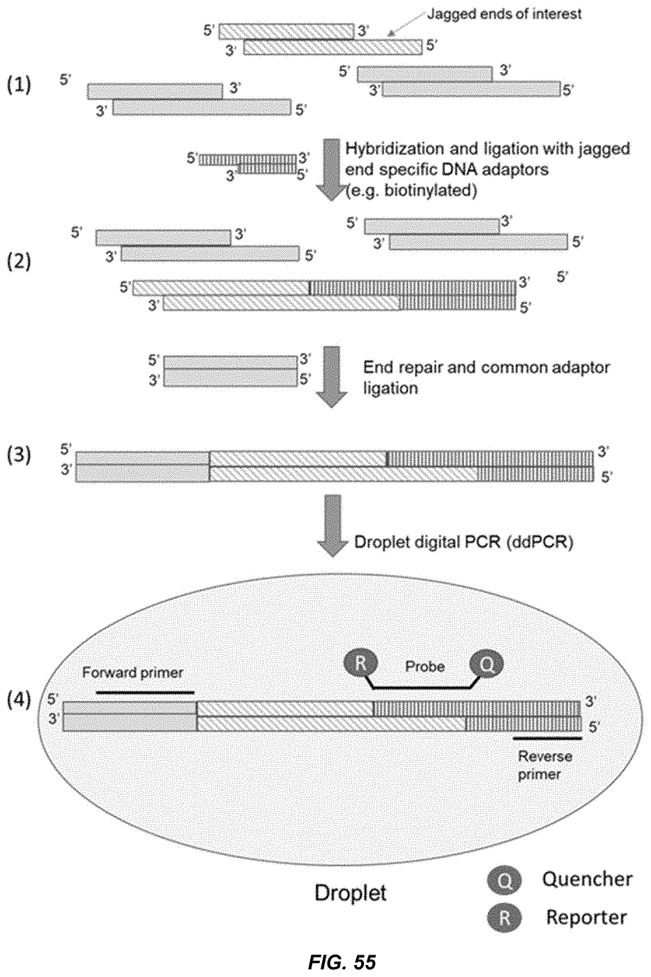

FIG. 55 illustrates an example of a method using droplet PCR to determine a certain number of jagged ends of interest according to some embodiments. In one embodiment for physical enrichment analysis, the jagged ends of interest for a molecule would be specifically ligated with an adaptor (namely jagged end specific adaptor (illustrated in step 1 and 2). The other end of the same molecule would become blunt after DNA end repair, which could be ligated with a universal adaptor (common adaptor) (illustrated in step 3). A molecule ligated with both common adaptor and jagged end specific adaptor were subjected to droplet digital PCR analysis (ddPCR) (illustrated in step 4). In one embodiment, such ddPCR analysis would utilize forward primer targeting the common adaptor, the probes with quencher and fluorescent reporter and reverse primer targeting the jagged end specific adaptor. Hence, the droplets containing the jagged ends of interest would result in positive readouts. In one embodiment, one or more different jagged ends were analyzed together, e.g., ratios or deviations between readouts of different jagged ends for practical applications.

In one variant embodiment, DNA end repair with 5 mC (or other ascertainable modified bases) and specific adaptors ligation could be combined in some applications for detecting jagged ends of interest.

### VIII. Viral DNA End Motif Analysis

Epstein-Barr virus (EBV) is an oncogenic virus that is associated with a number of malignancies, including nasopharyngeal carcinoma (NPC), Burkitt's lymphoma, Hodgkin's lymphoma, natural killer-T cell (NK-T cell) lymphoma, and post-transplant lymphoproliferative disease. EBV also causes a non-malignant disease called infectious mononucleosis. The presence of EBV DNA in a patient's plasma DNA pool was deemed as a biomarker for prognostication and monitoring for recurrence (Lo et al. Cancer Res. 1999; 59:5452-5455), which was furthered confirmed in a large-scale prospective study (Chan et al. N Engl J Med. 2017; 377:513-522). The fragment size of EBV DNA in plasma would be used for determining whether a patient with positive EBV DNA had NPC or not (Lam et al. Proc Natl Acad Sci USA. 2018; 115:E5115-E5124).

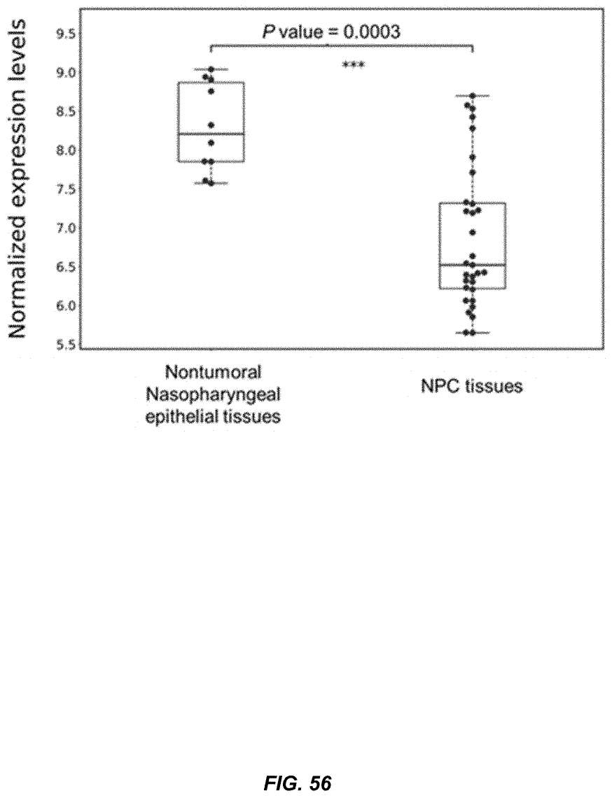

FIG. 56 shows a boxplot of expression levels of DNASE1L3 between non-tumoral nasopharyngeal epithelial tissues and NPC tissues, according to some embodiments. In this disclosure, we analyzed the DNASE1L3 expression level between NPC tissues and non-tumoral nasopharyngeal epithelial tissues according to a published microarray dataset (Sengupta et al. Cancer Res. 2006). We found that the DNASE1L3 expression level significantly decreased (e.g., downregulated) in NPC tissues (n=31) in comparison with non-tumoral nasopharyngeal epithelial tissues (n=10) (P value=0.0003, Mann-Whitney U test) (FIG. 56).

A. End Signature Analysis of Viral DNA Based on Differential Regulation of Nucleases

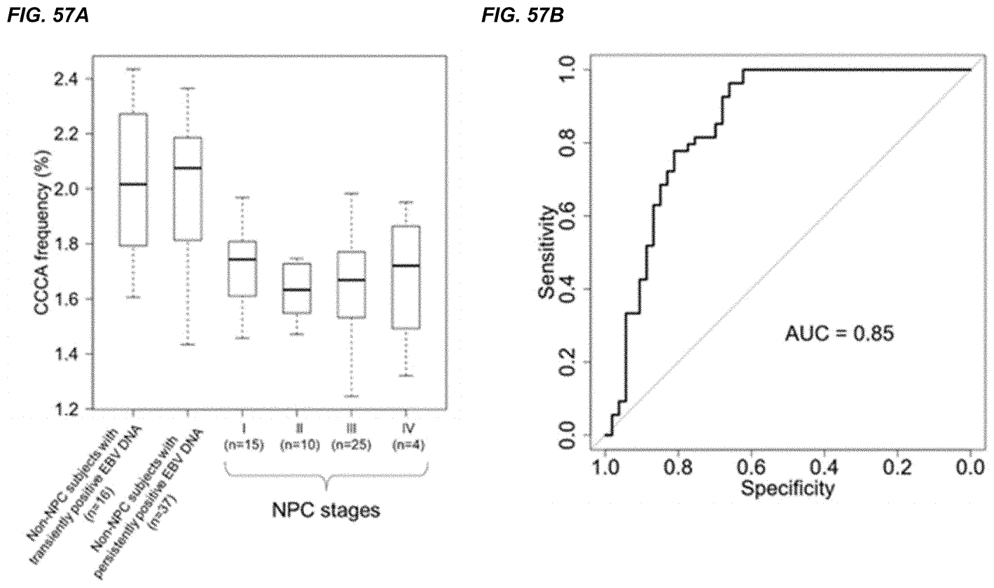

FIG. 57A shows a boxplot of DNASE1L3-associated end motif CCCA across different subjects with varying stages of nasopharyngeal carcinoma, and FIG. 57B shows an ROC curve depicting performance levels of end motif CCCA in differentiating EBV DNA positive subjects with and without NPC, according to some embodiments. Therefore, we used the DNASE1L3-associated end motif (e.g., CCCA) to classify cancer status for patients with positive EBV DNA. For an illustration purpose, we analyzed end signatures in plasma EBV DNA from those subjects with at least 1000 EBV DNA fragments in a previously published study (Lam et al. Proc Natl Acad Sci USA. 2018; 115:E5115-E5124). As shown in FIG. 57A, compared with patients without NPC (mean % CCCA: 2.01; range: 1.19-2.43), the percentage of DNASE1L3-associated end motif CCCA was significantly reduced (e.g., downregulated) in NPC groups (mean % CCCA: 1.68; range: 1.25-1.98) including patients with stages I, II, III, and IV (P value <0.0001, Mann Whitney U test). The AUC was 0.85 (FIG. 57B). These results suggested that the DNASE1L3-associated end motif could also be used as a biomarker for detecting patients with NPC.

In one embodiment, we could define nuclease-cutting signatures by using a permutation analysis to determine the combination of cutting signatures exhibiting the most discriminative power in differentiating EBV DNA positive patients with and without NPC. As an example, one could enumerate all combinations of frequency ratios between any two end motifs. There are 256 motifs, leading to 32,640. Among 32,640 frequency ratios between any two end motifs, the frequency ratio of the CCCG to TGGT end motif gave an AUC of 0.87, which was greater than AUC only based on CCCA %.

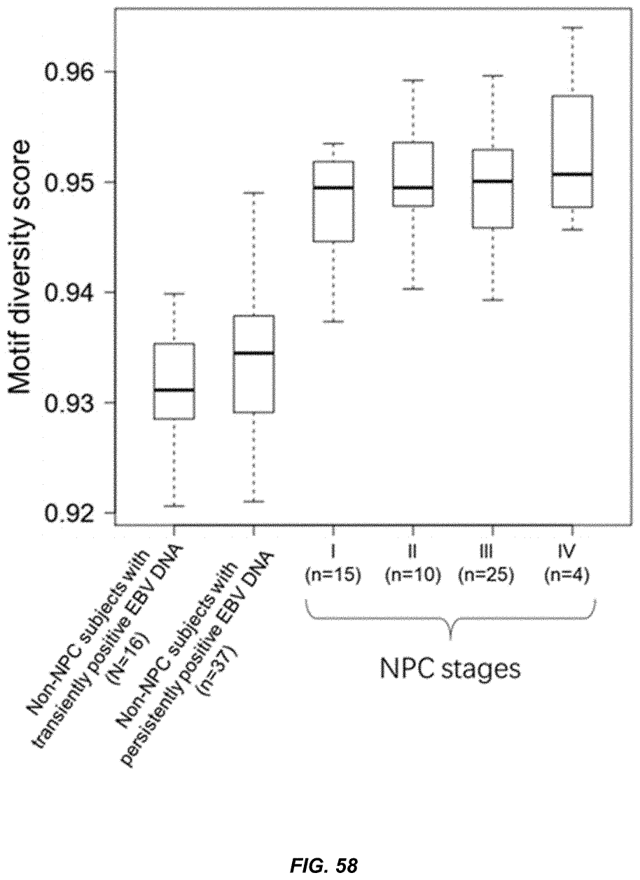

FIG. 58 shows a boxplot of motif diversity scores across different subjects with varying stages of nasopharyngeal carcinoma according to some embodiments. In one embodiment, the nucleases aberration would result in the skewness of end motifs. Therefore, the motif diversity would be changed accordingly. The motif diversity scores were aberrantly higher in patients with NPC (mean: 0.950; range: 0.937-0.966), compared with patient without NPC (mean: 0.933; range: 0.921-0.949) (FIG. 58) (P value <0.0001, Mann Whitney U test).

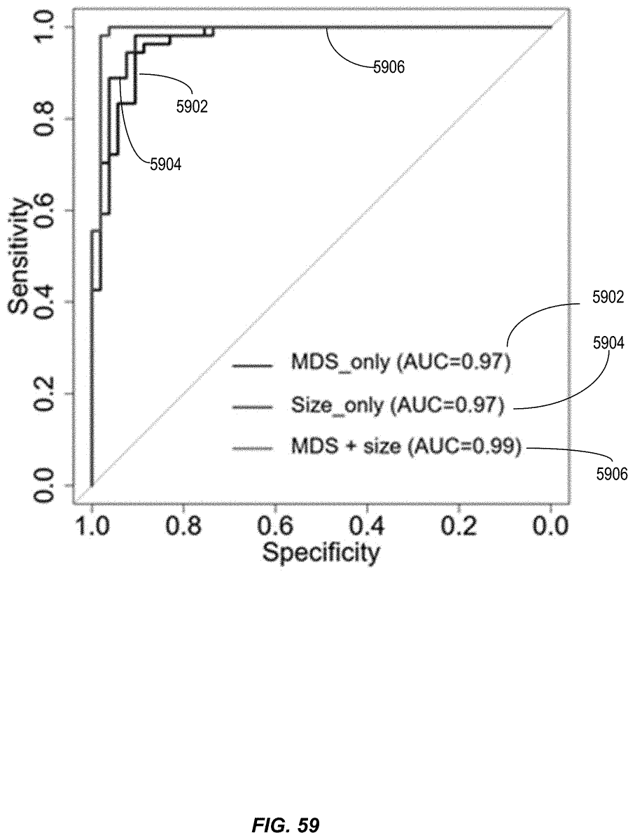

FIG. 59 shows ROC curves for assessing performance levels of combined MDS and size analysis according to some embodiments. In FIG. 59, MDS only line 5902 represents ROC curve for an analysis that used MDS, Size_only line 5904 represents an ROC curve for an analysis that used size ratio, and MDS+size line 5906 represents ROC curve for analysis that combined MDS and size. In one embodiment, MDS and size signals are combined to enhance the performance of cancer detection. FIG. 59 shows that the combined MDS and size analysis (AUC: 0.99) outperforms the analysis which only taking into account either MDS (AUC: 0.97) or size (AUC: 0.97).

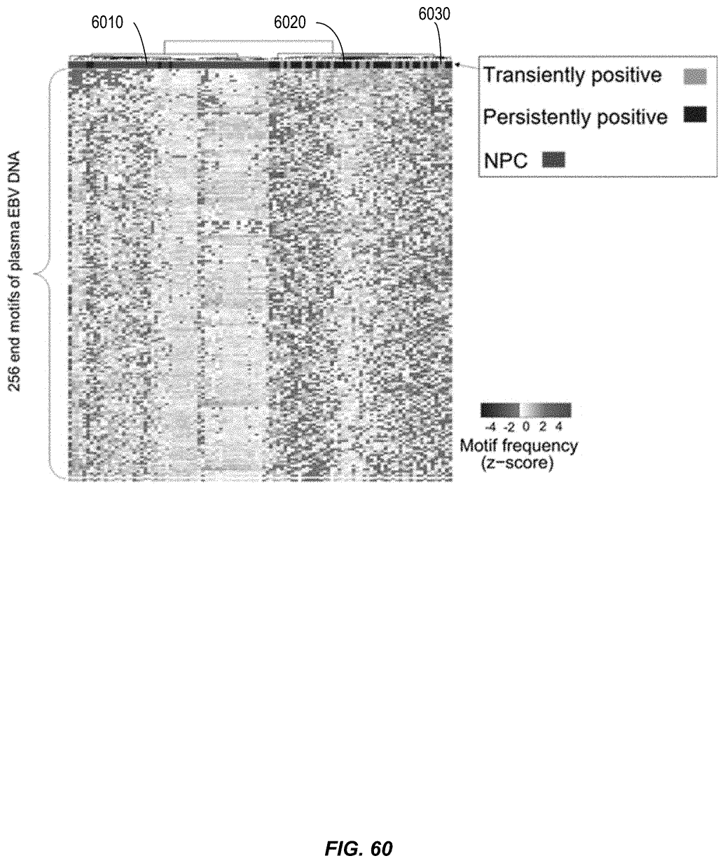

FIG. 60 shows a heatmap of 256 end motifs deduced from plasma EBV DNA fragments across patients with NPC (color 6010) and patients with transiently (color 6030) or persistently positive EBV DNA but without NPC (color 6020), according to some embodiments. As shown in FIG. 60, by taking advantage of patterns of 256 end motifs, patients with and without NPC could be clustered into two distinct groups, suggesting that in one embodiment one could use more than one end motifs to perform cancer detection. In another embodiment, one could employ different statistical approaches to selectively make use of a number end motifs, for example but not limited to, including logistic regression, support vector machines (SVM), decision tree, naïve Bayes classification, clustering algorithm, principal component analysis, singular value decomposition (SVD), t-distributed stochastic neighbor embedding (tSNE), artificial neural network, ensemble methods which construct a set of classifiers and then classify new data points by taking a weighted vote of their prediction.

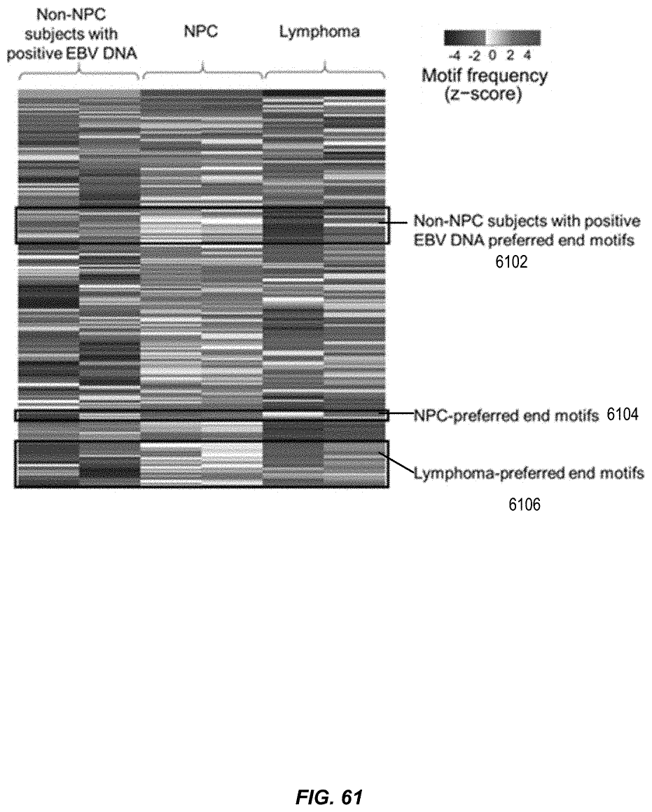

FIG. 61 shows a heatmap that identifies end motifs of plasma EBV DNA which were preferentially present in non-NPC subjects with positive EBV DNA according to some embodiments. In one embodiment, one could determine a series of end motifs that are preferentially present in a certain disease, which are referred to as disease preferred end motifs. For example, as shown in FIG. 61, one could identify the end motifs of plasma EBV DNA 6102 which were preferentially present in non-NPC subjects with positive EBV DNA, including but not limited to TCCC, TCCT, TCTT. One could identify the end motifs of plasma EBV DNA which were preferentially present in NPC subjects 6104, including but not limited to GCGC, GCGT, TTTA. One could identify the end motifs of plasma EBV DNA which were preferentially present in patients with lymphoma 6106, including but not limited to ATCT, ATCA, ATCC.

B. Methods for Determining a Level of Pathology Using End Signature Analysis of Viral DNA

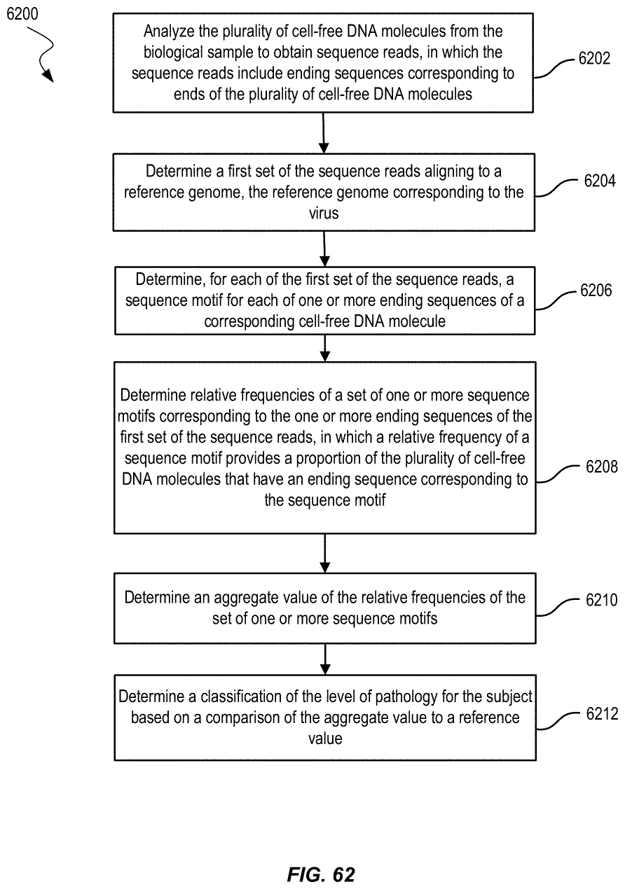

FIG. 62 is a flowchart illustrating a method of analyzing a biological sample with cell-free viral DNA molecules to determine a level of pathology in a subject from which the biological sample is obtained, in accordance to some embodiments. The biological sample includes a plurality of cell-free DNA molecules from the subject and a virus (e.g., EBV). The abnormality may be a pathology including cancer (e.g., NPC, HCC, lung cancer, breast cancer, gastric cancer, glioblastoma multiforme, pancreatic cancer, colorectal cancer, and/or head and neck squamous cell carcinoma) and an auto-immune disorder (e.g., systemic lupus erythematosus). In some instances, the abnormality in the biological sample is an abnormality of placental tissue (e.g., placental tissue detected in maternal plasma), including preeclampsia, preterm birth, fetal chromosomal aneuploidies, or fetal genetic disorders.

At step 6202, the plurality of cell-free DNA molecules from the biological sample are analyzed to obtain sequence reads. In some embodiments, the sequence reads include ending sequences corresponding to ends of the plurality of cell-free DNA molecules. The sequence reads can include ending sequences corresponding to ends of the plurality of cell-free DNA fragments. As examples, the sequence reads can be obtained using sequencing or probe-based techniques, either of which may including enriching, e.g., via amplification or capture probes.

The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids.

The sequencing may be targeted sequencing as described herein. For example, biological sample can be enriched for DNA fragments from a particular region. The enriching can include using capture probes that bind to a portion of, or an entire genome, e.g., as defined by a reference genome.

A statistically significant number of cell-free DNA molecules can be analyzed so as to provide an accurate determination of the fractional concentration. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed.

At step 6204, a first set of the sequence reads aligning to a reference genome are determined. In some embodiments, the reference genome corresponding to the virus.

At step 6206, for each of the first set of the sequence reads, a sequence motif is determined for each of one or more ending sequences of a corresponding cell-free DNA molecule. The sequence motifs can include N base positions (e.g., 1, 2, 3, 4, 5, 6, etc.). As examples, the sequence motif can be determined by analyzing the sequence read at an end corresponding to the end of the DNA fragment, correlating a signal with a particular motif (e.g., when a probe is used), and/or aligning a sequence read to a reference genome, e.g., as described in FIG. 1.

For example, after sequencing by a sequencing device, the sequence reads may be received by a computer system, which may be communicably coupled to a sequencing device that performed the sequencing, e.g., via wired or wireless communications or via a detachable memory device. In some implementations, one or more sequence reads that include both ends of the nucleic acid fragment can be received. The location of a DNA molecule can be determined by mapping (aligning) the one or more sequence reads of the DNA molecule to respective parts of the human genome, e.g., to specific regions. In other embodiments, a particular probe (e.g., following PCR or other amplification) can indicate a location or a particular end motif, such as via a particular fluorescent color. The identification can be that the cell-free DNA molecule corresponds to one of a set of sequence motifs.

At step 6208, relative frequencies of a set of one or more sequence motifs corresponding to the one or more ending sequences of the first set of the sequence reads are determined. In some embodiments, a relative frequency of a sequence motif provides a proportion of the plurality of cell-free DNA molecules that have an ending sequence corresponding to the sequence motif. The set of one or more sequence motifs can be identified using a reference set of one or more reference samples. The fractional concentration of clinically-relevant DNA need not be known for a reference sample, although genotypic differences may be determined so that differences between the end motifs of the clinically-relevant DNA and the other DNA (e.g., healthy DNA, maternal DNA, or DNA of a subject how received a transplanted organ) may be identified. Particular end motifs can be selected on the basis of the differences (e.g., to select the end motifs with the highest absolute or percentage difference). Examples of relative frequencies are described throughout the disclosure.

In some implementations, the sequence motifs include N base positions, where the set of one or more sequence motifs include all combinations of N bases. In one example, N can be an integer equal to or greater than two or three. The set of one or more sequence motifs can be a top M (e.g., 10) most frequent sequence motifs occurring in the one or more calibration samples or other reference sample not used for calibrating the fractional concentration.

At step 6210, an aggregate value of the relative frequencies of the set of one or more sequence motifs is determined. Example aggregate values are described throughout the disclosure, e.g., including an entropy value (a motif diversity score), a sum of relative frequencies, and a multidimensional data point corresponding to a vector of counts for a set of motifs (e.g., a vector 256 counts for 245 motifs of possible 4-mers or 64 counts for 64 motifs of possible 3-mers). When the set of one or more sequence motifs includes a plurality of sequence motifs, the aggregate value can include a sum of the relative frequencies of the set.

As an example, when the set of one or more sequence motifs includes a plurality of sequence motifs, the aggregate value can include a sum of the relative frequencies of the set. As another example, the aggregate value can correspond to a variance in the relative frequencies. For instance, the aggregate value can include an entropy term. The entropy term can include a sum of terms, each term including a relative frequency multiplied by a logarithm of the relative frequency. As another example, the aggregate value can include a final or intermediate output of a machine learning model, e.g., clustering model.

At step 6212, a classification of the level of pathology for the subject is determined based on a comparison of the aggregate value to a reference value. In some embodiments, the classification of the level of abnormality includes one of a plurality of stages of pathology (e.g., NPC).

### IX. Viral DNA Jagged-End Analysis

In some embodiments, a specified length of overhang between two DNA strands can be associated with an end-cutting signature of subjects having a particular viral-related disease (e.g., nasopharyngeal carcinoma caused by EBV). For a biological sample, a parameter that identifies an amount of DNA molecules having this property (e.g., the specified length of overhang) can be generated, and the parameter can be used to predict a viral-related condition of the subject (e.g., NPC).

A. Jagged-End Analysis of Viral DNA Based on Differential Regulation of Nucleases

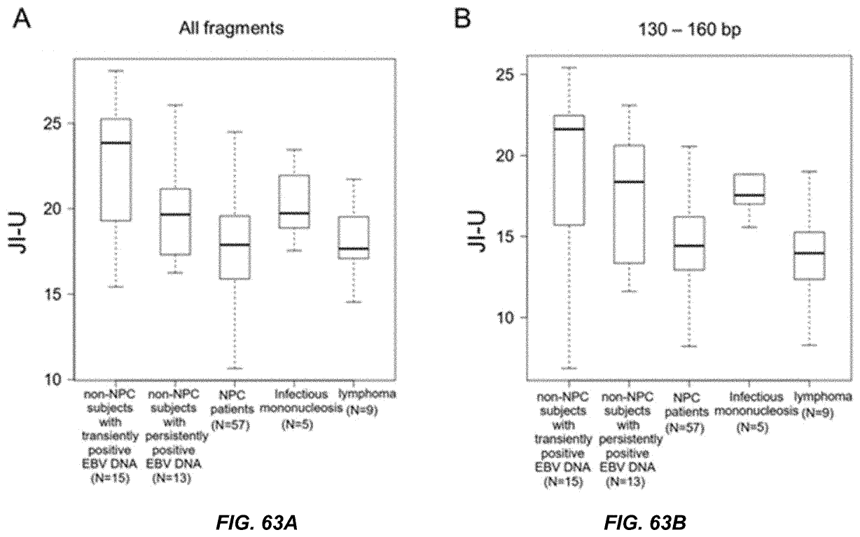

FIGS. 63A and 63B show boxplots of jaggedness index values deduced from unmethylated signals across different subjects according to some embodiments. We also explored the clinical utility of the jagged ends of plasma EBV DNA in this disclosure. As shown in FIG. 63A, using total plasma EBV DNA fragments which were sequenced, the quantity of jagged ends of EBV DNA in plasma was shown to be different between patients with cancers versus patients without cancer. The patients with cancers included NPC and lymphoma, and patients without cancer consisted of subjects with transiently positive EBV DNA and persistently positive EBV DNA as well as infectious mononucleosis. The jaggedness index value of plasma DNA EBV DNA in patients with cancers was 12.5% lower than non-NPC subjects with transiently positive EBV DNA and persistently positive EBV DNA (P value=0.0006, Mann Whitney U test). The jaggedness index value of plasma DNA EBV DNA in patients with cancers was 9.3% lower than patients with infectious mononucleosis (P value=0.06, Mann Whitney U test). However, the jaggedness index value of plasma DNA EBV DNA in patients with cancers was comparable with patients with lymphoma, only showing 1.3% difference (P value=1, Mann Whitney U test). These results suggested that the jagged ends of viral DNA would be a potential biomarker for differentiating patients with and without viral-driven cancers.

In another embodiment, as shown in FIG. 63B, the jaggedness index value of plasma EBV DNA could be deduced from those fragments between 130 and 160 bp in size to enhance the signal to noise ratios for differentiating EBV DNA positive patients with and without cancers. The jaggedness index value of plasma DNA EBV DNA in patients with cancers was 29.6% lower than non-NPC subjects with transiently positive EBV DNA and persistently positive EBV DNA (P value <0.0001, Mann Whitney U test). The jaggedness index value of plasma DNA EBV DNA in patients with cancers was 17.8% lower than patients with infectious mononucleosis (P value=0.01, Mann Whitney U test). Thus, using jaggedness deduced from those between a size range of 130 to 160 bp, an increased separation between NPC and non-NPC subjects with transiently positive EBV DNA and persistently positive EBV DNA was observed, suggesting size selection would increase the signal to noise ratio. However, the jaggedness index value of plasma DNA EBV DNA in patients with cancers was comparable with patients with lymphoma, only showing 3.3% difference (P value=0.56, Mann Whitney U test). In another embodiment, other size ranges could be used, for example but not limited to 50-80 bp, 60-90 bp, 70-100 bp, 80-110 bp, 90-120 bp, 100-130 bp, 110-140 bp, 120-150 bp, 140-170 bp, 150-180 bp, 160-190 bp, 170-200 bp, 180-210 bp, 190-220 bp, 200-230 bp, 210-240 bp, 220-250 bp, 230-260 bp, 230-270 bp, 250-280 bp, or a few combinations of different size ranges.

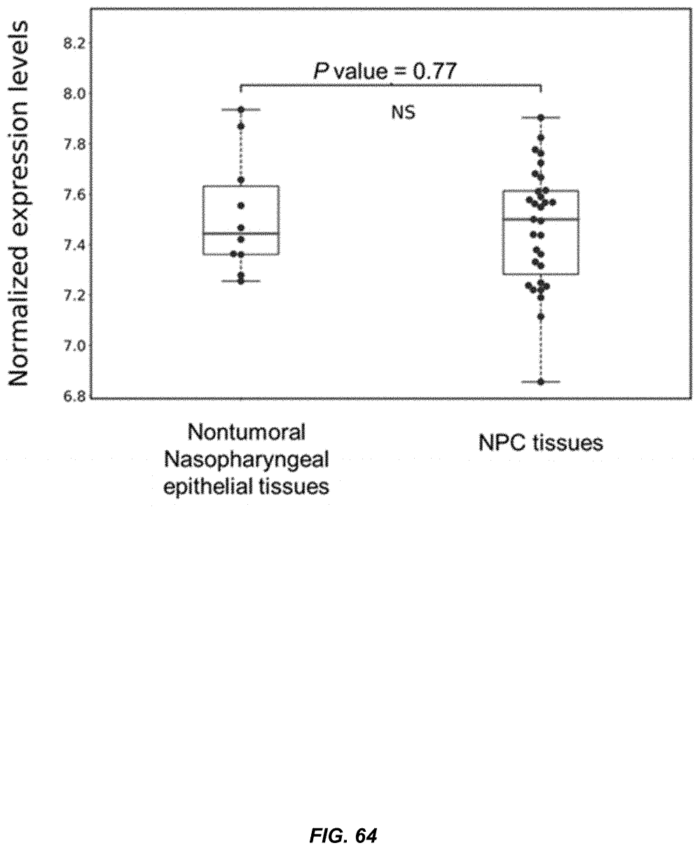

FIG. 64 shows a boxplot of DNASE1 expression levels between NPC tissues and non-tumoral nasopharyngeal epithelial tissues according to some embodiments. Referring back to FIG. 63, the decrease of jaggedness of plasma EBV DNA observed in patients with NPC, which was in contrast to the increase of jaggedness of plasma DNA in patient with HCC. One possible reason might be because the DNASE1 expression level showed no significant change between NPC tissues and non-tumoral nasopharyngeal epithelial tissues (P value=0.77, Mann Whitney U test) (FIG. 64), which was in contrast to the fact that the DNASE1 expression level was significantly upregulated in HCC tissues compared with adjacent non-tumoral liver tissues.

B. Methods for Determining a Level of Condition Using Jagged-End Analysis of Viral DNA

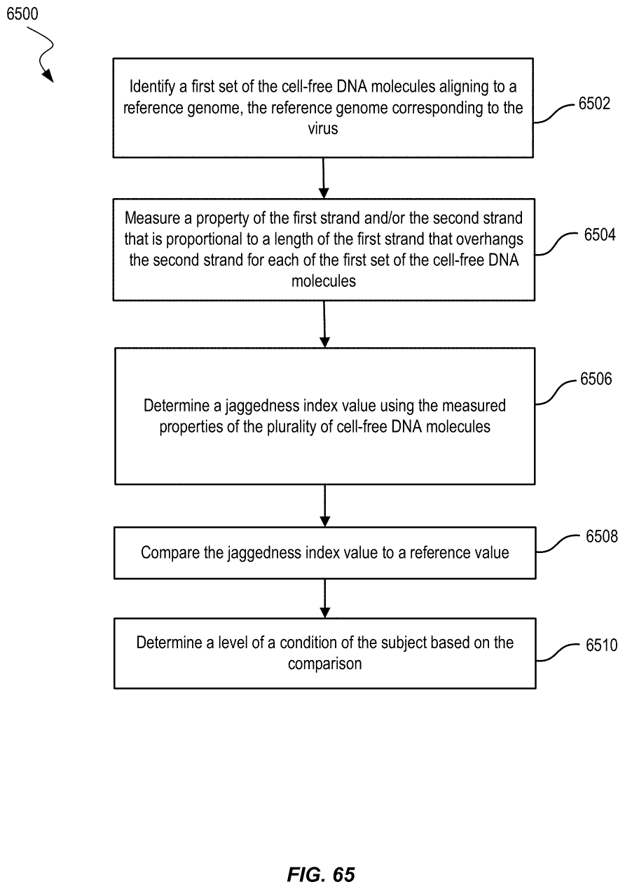

FIG. 65 is a flowchart illustrating a method of analyzing jagged ends of cell-free viral DNA molecules in a biological sample in accordance with some embodiments. In some instances, the biological sample includes a plurality of cell-free DNA molecules from the subject and a virus (e.g., an oncogenic virus), in which each of the plurality of cell-free DNA molecules being partially or completely double-stranded with a first strand having a first portion and a second strand. In some embodiments, the first portion of the first strand of at least some of the plurality of cell-free DNA molecules has no complementary portion from the second strand, is not hybridized to the second strand, and is at a first end of the first strand. In some instances, the first is a 5′ end.

At step 6502, a first set of the cell-free DNA molecules aligning to a reference genome is identified, in which the reference genome corresponds to the virus. The reads may be aligned to a reference genome. The plurality of nucleic acid molecules may be reads within a certain distance range relative to a transcription start site.

At step 6504, a property of the first strand and/or the second strand that is proportional to a length of the first strand that overhangs the second strand is measured for each of the first set of the cell-free DNA molecules. For example, a measured property includes a higher methylation level of the first strand, in which the higher methylation level is correlated with a longer length of the first strand that overhangs the second strand. In another example, a measured property includes a lower methylation level of the first strand, in which the lower methylation level is correlated with a longer length of the first strand that overhangs the second strand. In some instances, the property is a methylation status at one or more sites at end portions of the first strands and/or second strands of each of the plurality of nucleic acid molecules. In other instances, the property is a length of the first strand and/or the second strand that is proportional to the length of the first strand that overhangs the second strand.

At step 6506, a jaggedness index value is determined using the measured properties of the plurality of cell-free DNA molecules. In some embodiments, the jaggedness index value provides a collective measure that a strand overhangs another strand in the plurality of cell-free DNA molecules. In some instances, the jaggedness index value includes a methylation level over the plurality of nucleic acid molecules at one or more sites of end portions of the first strands and/or second strands. In some embodiments, the jaggedness index value corresponds to the measured properties of the plurality of the cell-free DNA molecules having size within a specified range, e.g., 130 to 160 bps (See FIG. 49B).

If the first plurality of nucleic acid molecules are in a specified size range, methods may include measuring the property of each nucleic acid molecule of a second plurality of nucleic acid molecules. The second plurality of nucleic acid molecules may have sizes with a second specified size range. Determining the jaggedness index value may include calculating a ratio using the measured properties of the first plurality of nucleic acid molecules and the measured properties of the second plurality of nucleic acid molecules. The jaggedness index value may include the jagged end ratio or the overhang index ratio described herein.

At step 6508, the jaggedness index value is compared to a reference value. The reference value or the comparison may be determined using machine learning with training data sets. The comparison may be used to determine different information regarding the biological sample or the individual.

At step 6510, a level of a condition of the subject is determined based on the comparison. The condition may include a disease, a disorder, or a pregnancy. The condition may be cancer, an auto-immune disease, a pregnancy-related condition, or any condition described herein. As examples, cancer may include nasopharyngeal carcinoma (NPC), hepatocellular carcinoma (HCC), colorectal cancer (CRC), leukemia, lung cancer, breast cancer, prostate cancer or throat cancer. The auto-immune disease may include systemic lupus erythematosus (SLE). Various data below provides examples for determined a level of a condition.

In some instances, the reference value is determined using one or more reference samples of subjects that have the condition. As another example, the reference value is determined using one or more reference samples of subjects that do not have the condition. Multiple reference values can be determined from the reference samples, potentially with the different reference values distinguishing between different levels of the condition.

The process may include determining a fraction of clinically-relevant DNA in a biological sample based on the comparison. Clinically-relevant DNA may include fetal DNA, tumor-derived DNA, or transplant DNA. The reference value may be obtained using nucleic acid molecules from one or more reference subjects having a known fraction of clinically-relevant DNA. Methods for determining the fraction of clinically-relevant DNA may include treating the plurality of nucleic acid molecules by a protocol before measuring the property of the first strand and/or the second strand. The nucleic acid molecules from one or more reference subjects may be treated by the same protocol as the plurality of nucleic acid molecules having the property measured.

Calibration data points can include a measured jaggedness index value and a measured/known fraction of the clinically-relevant DNA. The measured jaggedness index value for any sample whose fraction can be measured via another technique (e.g., using a tissue-specific allele) can be correspond to a reference value. As another example, a calibration curve (function) can be fit to the calibration data points, and the reference value can correspond to a point on the calibration curve. Thus, a measured jaggedness index value of a new sample can be input into the calibration function, which can output the faction of the clinically-relevant DNA.

### X. Treatment

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### XI. Example Systems

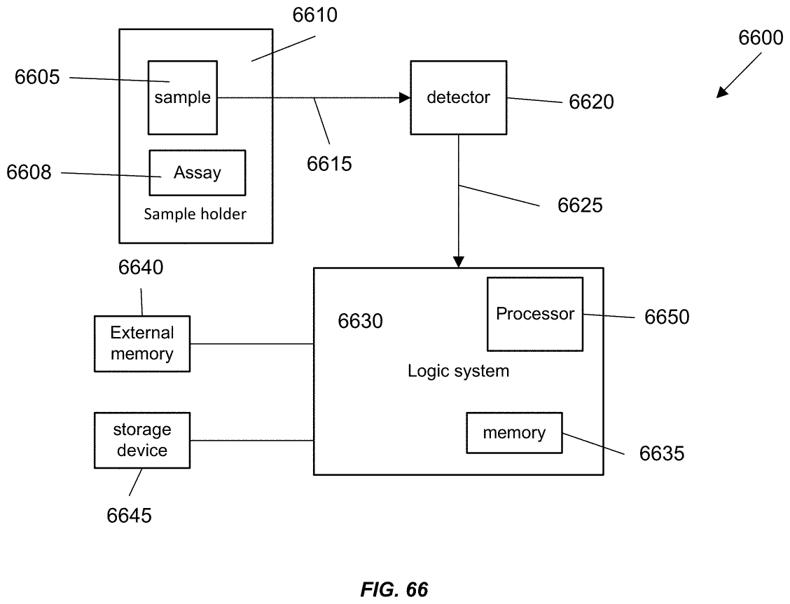

FIG. 66 illustrates a measurement system 6600 according to an embodiment of the present invention. The system as shown includes a sample 6605, such as cell-free DNA molecules within a sample holder 6610, where sample 6605 can be contacted with an assay 6608 to provide a signal of a physical characteristic 6615. An example of a sample holder can be a flow cell that includes probes and/or primers of an assay or a tube through which a droplet moves (with the droplet including the assay). Physical characteristic 6615 (e.g., a fluorescence intensity, a voltage, or a current), from the sample is detected by detector 6620. Detector 6620 can take a measurement at intervals (e.g., periodic intervals) to obtain data points that make up a data signal. In one embodiment, an analog-to-digital converter converts an analog signal from the detector into digital form at a plurality of times. Sample holder 6610 and detector 6620 can form an assay device, e.g., a sequencing device that performs sequencing according to embodiments described herein. A data signal 6625 is sent from detector 6620 to logic system 6630. Data signal 6625 may be stored in a local memory 6635, an external memory 6640, or a storage device 6645.

Logic system 6630 may be, or may include, a computer system, ASIC, microprocessor, etc. It may also include or be coupled with a display (e.g., monitor, LED display, etc.) and a user input device (e.g., mouse, keyboard, buttons, etc.). Logic system 6630 and the other components may be part of a stand-alone or network connected computer system, or they may be directly attached to or incorporated in a device (e.g., a sequencing device) that includes detector 6620 and/or sample holder 6610. Logic system 6630 may also include software that executes in a processor 6650. Logic system 6630 may include a computer readable medium storing instructions for controlling measurement system 6600 to perform any of the methods described herein. For example, logic system 6630 can provide commands to a system that includes sample holder 6610 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

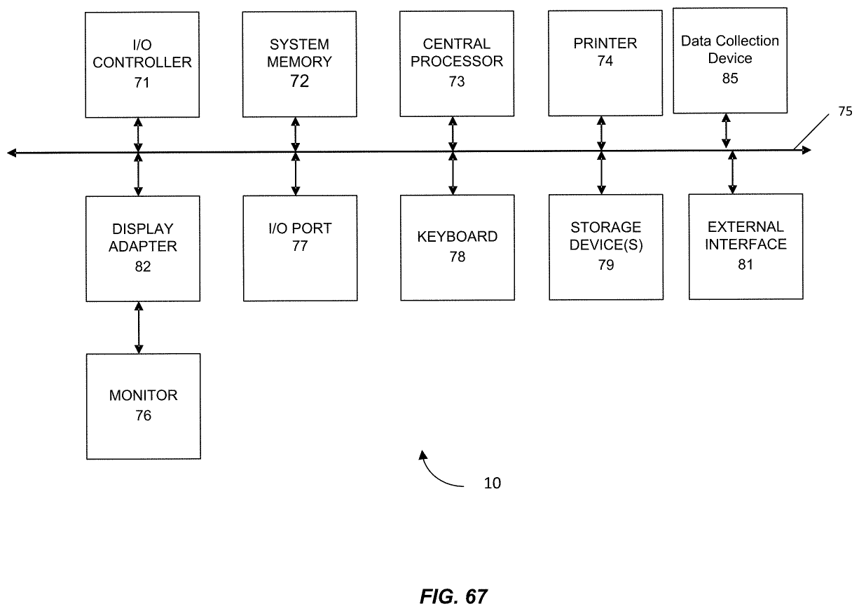

Any of the computer systems mentioned herein may utilize any suitable number of subsystems. Examples of such subsystems are shown in FIG. 67 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 67 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

1. A method of classifying a level of abnormality in a biological sample of a subject, the method comprising:
identifying that a first nuclease is differentially regulated in abnormal cells of one or more tissue types relative to a normal tissue of the one or more tissue types;
determining that the first nuclease preferentially cuts DNA into DNA molecules having a first sequence end signature relative to other sequence end signatures;
analyzing a plurality of cell-free DNA molecules from the biological sample to obtain sequence reads, wherein the sequence reads include ending sequences corresponding to ends of the plurality of cell-free DNA molecules;
identifying a first set of the sequence reads, wherein each sequence read of the first set of the sequence reads includes an ending sequence corresponding to the first sequence end signature;
determining a first amount of the first set of the sequence reads;
determining a first parameter using the first amount of the sequence reads; and
determining a classification of the level of abnormality in the one or more tissue types in the biological sample using the first parameter.

2. The method of claim 1, wherein the determination of the classification of the level of abnormality is based on a comparison between the first parameter and a reference value.

3. The method of claim 1, further comprising:
identifying that a second nuclease is differentially regulated in the abnormal cells of the one or more tissue types relative to the normal tissue of the one or more tissue types;
determining that the second nuclease preferentially cuts the DNA into DNA molecules having a second sequence end signature relative to the other sequence end signatures;
identifying a second set of the sequence reads, wherein each sequence read of the second set of the sequence reads includes an ending sequence corresponding to the second sequence end signature;
determining a second amount of the second set of the sequence reads; and
determining a second parameter using the second amount of the sequence reads, wherein the classification of the level of abnormality in the one or more tissue types in the biological sample is determined further using the second parameter.

4. The method of claim 3, wherein the first nuclease is upregulated and the second nuclease is downregulated in the abnormal cells relative to the normal tissue of the one or more tissue types.

5. The method of claim 1, further comprising:
identifying that a second nuclease is differentially regulated in the abnormal cells of the one or more tissue types relative to the normal tissue of the one or more tissue types;
determining that the second nuclease preferentially cuts the DNA into DNA molecules having a second sequence end signature relative to the other sequence end signatures;
identifying a second set of the sequence reads, wherein each sequence read of the second set of the sequence reads includes an ending sequence corresponding to the second sequence end signature; and
determining a second amount of the second set of the sequence reads, wherein the second amount is used for determining the first parameter.

6. The method of claim 5, wherein the first nuclease is upregulated and the second nuclease is downregulated in the abnormal cells relative to the normal tissue of the one or more tissue types.

7. The method of claim 1, wherein the one or more tissue types include fetal tissue.

8. The method of claim 1, wherein the subject is a pregnant female, and the one or more tissue types include placental tissue detected in maternal plasma.

9. The method of claim 8, wherein the abnormality includes preeclampsia, preterm birth, fetal chromosomal aneuploidies, or fetal genetic disorders.

10. The method of claim 1, further comprising:
analyzing a biological sample of another subject, wherein the other subject is a different organism from the subject; and
determining, based on the biological sample of the other subject, that the first nuclease preferentially cuts the DNA into DNA molecules having the first sequence end signature.

11. The method of claim 1, wherein the abnormality is a pathology.

12. The method of claim 11, wherein the pathology is cancer, wherein the cancer includes hepatocellular carcinoma, lung cancer, breast cancer, gastric cancer, glioblastoma multiforme, pancreatic cancer, colorectal cancer, nasopharyngeal carcinoma, or head and neck squamous cell carcinoma, or any combination thereof.

13. The method of claim 11, wherein the classification is one of a plurality of stages of the pathology.

14. The method of claim 11, wherein the pathology is an auto-immune disorder.

15. The method of claim 14, wherein the auto-immune disorder is systemic lupus erythematosus.

16. A method of estimating a fractional concentration of clinically-relevant DNA molecules in a biological sample of a subject, the method comprising:
identifying that a first nuclease is differentially regulated in a target tissue type relative to at least one other tissue type of a plurality of tissue types, wherein the clinically-relevant DNA molecules are from the target tissue type;
determining that the first nuclease preferentially cuts DNA into DNA molecules having a first sequence end signature relative to other sequence end signatures;
analyzing a plurality of cell-free DNA molecules from the biological sample to obtain sequence reads, wherein the biological sample includes a mixture of cell-free DNA molecules from the plurality of tissue types, and wherein the sequence reads include ending sequences corresponding to ends of the plurality of the cell-free DNA molecules;
identifying a first set of the sequence reads, wherein each sequence read of the first set of the sequence reads includes an ending sequence corresponding to the first sequence end signature;
determining a first amount of the first set of the sequence reads;
determining a first parameter using the first amount of the sequence reads; and
estimating the fractional concentration of the clinically-relevant DNA molecules in the biological sample using the first parameter and one or more calibration values determined from one or more calibration samples whose fractional concentration of the clinically-relevant DNA molecules are known.

17. The method of claim 16, wherein the clinically-relevant DNA molecules include fetal DNA, tumor DNA, or DNA of a transplanted organ.

18. A method of determining a characteristic of a target tissue type, the method comprising:
identifying that a first nuclease is differentially regulated in the target tissue type relative to at least one other tissue type of a plurality of tissue types;
determining that the first nuclease preferentially cuts DNA into DNA molecules having a first sequence end signature relative to other sequence end signatures;
analyzing a plurality of cell-free DNA molecules from a biological sample to obtain sequence reads, wherein the biological sample includes a mixture of cell-free DNA molecules from the plurality of tissue types, and wherein the sequence reads include ending sequences corresponding to ends of the plurality of cell-free DNA molecules;
identifying a first set of the sequence reads, wherein each sequence read of the first set of the sequence reads includes an ending sequence corresponding to the first sequence end signature;
determining a first amount of the first set of the sequence reads;
determining a first parameter for the first amount of the sequence reads; and
estimating a first value for the characteristic of the target tissue type using the first parameter and one or more calibration values determined from one or more calibration samples whose values for the characteristic are known.

19. The method of claim 16, further comprising:
identifying that a second nuclease is differentially regulated in the target tissue type;
determining that the second nuclease preferentially cuts the DNA into DNA molecules having a second sequence end signature relative to the other sequence end signatures;
identifying a second set of the sequence reads, wherein each sequence read of the second set of the sequence reads includes an ending sequence corresponding to the second sequence end signature;
determining a second amount of the second set of the sequence reads; and
determining a second parameter using the second amount, wherein the fractional concentration is further estimated using the second parameter.

20. The method of claim 19, wherein the first nuclease is upregulated and the second nuclease is downregulated in the target tissue type relative to a normal tissue of the plurality of tissue types.

21. The method of claim 19, wherein the fractional concentration is estimated by comparing the second parameter to another reference value.

22. The method of claim 16, further comprising:
identifying that a second nuclease is differentially regulated in the target tissue type relative to the at least one other tissue type of the plurality of tissue types;
determining that the second nuclease preferentially cuts the DNA into DNA molecules having a second sequence end signature relative to the other sequence end signatures;
identifying a second set of the sequence reads, wherein each sequence read of the second set of the sequence reads includes an ending sequence corresponding to the second sequence end signature; and
determining a second amount of the second set of the sequence reads, wherein the second amount is used for determining the first parameter.

23. The method of claim 22, wherein the first nuclease is upregulated and the second nuclease is downregulated in the target tissue type relative to at least one other tissue type.

24. The method of claim 16, further comprising:
analyzing a biological sample of another subject, wherein the other subject is a different organism from the subject; and
determining, based on the biological sample of the other subject, that the first nuclease preferentially cuts the DNA into DNA molecules having the first sequence end signature.

25. The method of claim 16, wherein the target tissue type is liver or hematopoietic cells.

26. The method of claim 16, wherein the target tissue type is fetal tissue.

27. The method of claim 16, wherein the target tissue type is an organ that has cancer.

28. The method of claim 16, wherein the subject is a pregnant female, and wherein the target tissue type is placental tissue.

29. The method of claim 18, wherein the target tissue type is placental tissue, and wherein the characteristic of the placental tissue includes a gestational age of a pregnant subject.

30. The method of claim 16, wherein using the first parameter and the one or more calibration values includes comparing the first parameter to the one or more calibration values.

31. The method of claim 30, wherein comparing the first parameter to the one or more calibration values includes comparing the first parameter to a calibration curve that includes the one or more calibration values.

32. The method of claim 31, wherein comparing the first parameter to the calibration curve includes inputting the first parameter to a calibration function that represents the calibration curve.

33. The method of claim 1, wherein the first nuclease includes Deoxyribonuclease 1 Like 3 (DNASE1L3), Deoxyribonuclease 1 (DNASE1), DNA fragmentation factor subunit beta (DFFB), Three Prime Repair Exonuclease 1 (TREX1), Apoptosis Enhancing Nuclease (AEN), Exonuclease 1 (EXO1), Deoxyribonuclease 2 (DNASE2), Endonuclease G (ENDOG), Apurinic/Apyrimidinic Endodeoxyribonuclease 1 (APEX1), Flap Structure-Specific Endonuclease 1 (FEN1), Deoxyribonuclease 1 Like 1 (DNASE1L1), Deoxyribonuclease 1 Like 2 (DNASE1L2), or Exo/Endonuclease G (EXOG).

34. The method of claim 33, wherein:
the first nuclease is the DNASE1L3; and
the first sequence end signature corresponds to a nucleotide end sequence that includes CCCA or CGTA.

35. The method of claim 33, wherein:
the first nuclease is the DFFB; and
the first sequence end signature corresponds to a nucleotide end sequence that includes AAAA or AAAT.

36. The method of claim 33, wherein:
the first nuclease is the DNASE1; and
the first sequence end signature corresponds to a nucleotide end sequence that includes TAAT.

37. The method of claim 3, wherein the second nuclease includes Deoxyribonuclease 1 Like 3 (DNASE1L3), Deoxyribonuclease 1 (DNASE1), DNA fragmentation factor subunit beta (DFFB), Three Prime Repair Exonuclease 1 (TREX1), Apoptosis Enhancing Nuclease (AEN), Exonuclease 1 (EXO1), Deoxyribonuclease 2 (DNASE2), Endonuclease G (ENDOG), Apurinic/Apyrimidinic Endodeoxyribonuclease 1 (APEX1), Flap Structure-Specific Endonuclease 1 (FEN1), Deoxyribonuclease 1 Like 1 (DNASE1L1), Deoxyribonuclease 1 Like 2 (DNASE1L2), or Exo/Endonuclease G (EXOG).

38. The method of claim 1, wherein analyzing the plurality of cell-free DNA molecules includes sequencing the plurality of cell-free DNA molecules to obtain the sequence reads.

39. The method of claim 1, wherein the first parameter is a ratio between the first amount and another amount of the sequence reads.

40-150. (canceled)
